(19) United States
(12) Patent Application Publication
(10) Pub. No.: US 2026/0168987 A1
(43) Pub. Date: Jun. 18, 2026

(54) OBLIQUE LINE SCANNER SYSTEMS AND METHODS FOR HIGH THROUGHPUT SINGLE MOLECULE TRACKING IN LIVING CELLS

(71) Applicant: EIKON THERAPEUTICS, INC., Millbrae, CA (US)

(72) Inventors: Daniel ANDERSON, Millbrae, CA (US); David MCSWIGGEN, Millbrae, CA (US); Jaclyn HO, Millbrae, CA (US); Amine DRIOUCHI, Millbrae, CA (US); Russell BERMAN, Millbrae, CA (US); Brian MARGOLIN, Millbrae, CA (US)

(73) Assignee: EIKON THERAPEUTICS, INC., Millbrae, CA (US)

(21) Appl. No.: 19/244,379

(22) Filed: Jun. 20, 2025

Related U.S. Application Data
(63) Continuation of application No. PCT/US2023/085594, filed on Dec. 21, 2023.
(60) Provisional application No. 63/476,952, filed on Dec. 22, 2022.
(60) Provisional application No. 63/476,942, filed on Dec. 22, 2022.

(51) Int. Cl. G01N 33/50; G01N 21/64; G01N 33/68; G02B 21/00; G02B 21/26

(52) U.S. Cl. CPC G01N 33/5029; G01N 21/6458; G01N 33/6845; G02B 21/0032; G02B 21/0076; G02B 21/26

(57) ABSTRACT

High Throughput Single Molecule Tracking (htSMT) systems and methods are described wherein the htSMT workflows are adapted to characterize both known and novel pathway contributions to interaction networks in live cells, such as protein signaling interaction networks.

### CROSS-REFERENCE TO RELATED APPLICATIONS

This application is a continuation of International Patent Application No. PCT/US2023/085594, filed Dec. 21, 2023, which claims priority to U.S. Provisional Application No. 63/476,952, filed Dec. 22, 2022, and U.S. Provisional Application No. 63/476,942, filed Dec. 22, 2022, the contents of each of which are incorporated herein by reference herein in their entirety.

### TECHNICAL FIELD

The subject matter described herein relates to a platform to track single molecules within complex systems.

### BACKGROUND

The movement of proteins within the crowded environment of living cells are profoundly influenced by interactions with their surroundings. Single molecule tracking (SMT) is one method for capturing protein movement as a reporter of activity. In SMT, a fluorescent protein of interest is imaged at high spatiotemporal resolution to track its movement in a complex system, e.g., a live cell. The information embedded in these tracks has been used to investigate diverse cellular phenomena including protein-protein interactions, e.g., interactions mediating signal transduction, inter-organelle communication, nuclear organization, and transcription regulation. The application of SMT techniques has been limited in scale, however, and therefore mainly used to address specific mechanistic hypotheses. For example, SMT has not been adapted to a throughput setting that would enable systems-level screening or drug discovery.

### SUMMARY OF THE INVENTION

In a first aspect, the present disclosure is directed to a method of identifying a biological interaction between a compound and a target fluorescent protein in a live cell comprising: (a) contacting a sample comprising a population of live cells with the compound, wherein the live cells comprise target fluorescent protein; (b) tracking the movement of a plurality of individual target fluorescent proteins in a plurality of live cells in the sample, wherein said tracking comprises: (i) illuminating a field of view in a sample plane disposed within the sample with a light beam to cause fluorescence by a subset of the target fluorescent proteins in the live cells; and (ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension; and (c) determining a change in the movement of at a target fluorescent protein in the presence of the compound, wherein a change in the movement of the target fluorescent protein in the presence of the compound relative to the movement of the target fluorescent protein in the absence of the compound identifies a biological interaction between the compound and the target fluorescent protein.

In an interrelated aspect, the present disclosure is directed to a method of identifying a biological interaction between a compound and a target fluorescent protein in a live cell comprising: (a) contacting a sample comprising a population of live cells with the compound, wherein the live cells comprise target fluorescent protein; (b) tracking the movement of a plurality of individual target fluorescent protein in a plurality of live cells in the sample, wherein said tracking comprises: (i) illuminating a field of view in a sample plane disposed within the sample with a light beam to cause fluorescence by a subset of the target fluorescent proteins in the live cells, wherein the subset of the target fluorescent proteins produces up to about 1,000,000 molecular trajectories in a single detected field of view; and (ii) detecting the fluorescence from a plurality of the target fluorescent proteins in the detected field of view of the sample plane via a detector device, wherein the detected field of view has a size of about 150 μm to about 250 μm in a first dimension by about 100 μm to about 210 μm in a second dimension; and (c) determining a change in the movement of a target fluorescent protein in the presence of the compound, wherein a change in the movement of the target fluorescent protein in the presence of the compound relative to the movement of the target fluorescent protein in the absence of the compound identifies a biological interaction between the compound and the target fluorescent protein.

In an interrelated aspect, the present disclosure is directed to a method of identifying a biological interaction between a compound and a target fluorescent protein in a live cell comprising: (a) contacting a sample comprising a population of live cells with the compound, wherein the live cells comprise target fluorescent proteins; (b) tracking the movement of a plurality of individual target fluorescent proteins in a plurality of live cells in the sample, wherein said tracking comprises: (i) illuminating a field of view in a sample plane disposed within the sample with a light beam to cause fluorescence by a subset of the target fluorescent proteins in the live cells, wherein the field of view is illuminated using a 0.1 to 1 msec stroboscopic laser pulse; and (ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension; and (c) determining a change in the movement of the target fluorescent proteins in the presence of the compound, wherein a change in the movement of a target fluorescent protein in the presence of the compound relative to the movement of the target fluorescent protein in the absence of the compound identifies a biological interaction between the compound and the target fluorescent protein.

In an interrelated aspect, the present disclosure is directed to a method of identifying a biological interaction between a compound and a target fluorescent protein in a live cell comprising: (a) contacting a sample comprising a population of live cells with the compound, wherein the live cells comprise target fluorescent proteins; (b) tracking the movement of a plurality of individual target fluorescent proteins in a plurality of live cells in the sample, wherein said tracking comprises: (i) illuminating a field of view in a sample plane disposed within the sample with a light beam to cause fluorescence by a subset of the target fluorescent proteins in the live cells; (ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension; and (iii) detecting the fluorescence from a plurality of the target fluorescent proteins in the detected field of view of the sample plane at a rate of more than about 100,000 detected field of views per day; and (c) determining a change in the movement of a target fluorescent protein in the presence of the compound, wherein a change in the movement of the target fluorescent proteins in the presence of the compound relative to the movement of target fluorescent protein in the absence of the compound identifies a biological interaction between the compound and the target fluorescent protein.

In an interrelated aspect, the present disclosure is directed to a method of identifying a biological interaction between a compound and a target fluorescent protein in a live cell comprising: (a) contacting a sample comprising a population of live cells with the compound, wherein the live cells comprise the fluorescently labeled target protein; (b) tracking the motion of a plurality of individual target fluorescent proteins in a plurality of live cells in the sample, wherein said tracking comprises: (i) illuminating a field of view in a sample plane disposed within the sample with a light beam to cause fluorescence by a subset of the target fluorescent proteins in the live cells; (ii) detecting the fluorescence from a plurality of the target fluorescent proteins in a detected field of view of the imaging plane via a detector device, wherein the detected field of view has a size of about 150 μm to about 250 μm in a first dimension by about 100 μm to about 210 μm in a second dimension; and (iii) achieving, based on a single field of view, a z-factor of >0.5; and (c) determining a change in the motion of the fluorescently labeled target protein in the presence of the compound, wherein a change in the movement of the target fluorescent protein in the presence of the compound relative to the motion of the target fluorescent protein in the absence of the compound identifies a biological interaction between the compound and the target fluorescent protein.

In an interrelated aspect, the present disclosure is directed to a method of identifying a biological interaction between a compound and a target fluorescent protein in a live cell comprising: (a) contacting a sample comprising a population of live cells with the compound, wherein the live cells comprise target fluorescent proteins; (b) tracking the movement of a plurality of individual target fluorescent proteins in a plurality of live cells in the sample, wherein said tracking comprises: (i) illuminating a field of view in a sample plane disposed within the sample with a light beam to cause fluorescence by a subset of the target fluorescent proteins in the live cells; and (ii) detecting the fluorescence from a plurality of the target fluorescent proteins in a detected field of view of the sample plane via a detector device, wherein the field of view is detected with a frame rate up to about 2000 Hz and wherein the detected field of view has a size of about 150 μm to about 250 μm in a first dimension by about 100 μm to about 210 μm in a second dimension; and (c) determining a change in the movement of the fluorescently labeled target protein in the presence of the compound, wherein a change in the movement of the target fluorescent protein in the presence of the compound relative to the motion of the target fluorescent protein in the absence of the compound identifies a biological interaction between the compound and the target fluorescent protein.

In an interrelated aspect, the present disclosure is directed to a method of identifying a biological interaction between a compound and a target fluorescent protein in a live cell comprising: (a) contacting a sample comprising a population of live cells with the compound, wherein the live cells comprise target fluorescent proteins; (b) tracking the movement of a plurality of individual target fluorescent proteins in a plurality of live cells in the sample, wherein said tracking comprises: (i) illuminating a field of view in a sample plane disposed within the sample with a light beam to cause fluorescence by a subset of the target fluorescent proteins in the live cells; and (ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension and wherein equal to or greater than 95% of the detected field of view achieves sufficient laser illumination for tracking protein movement; and (c) determining a change in the movement of the fluorescently labeled target protein in the presence of the compound, wherein a change in the movement of the target fluorescent protein in the presence of the compound relative to the motion of the target fluorescent protein in the absence of the compound identifies a biological interaction between the compound and the target fluorescent protein.

In certain instances of the above aspects, the average change in movement of the target fluorescent proteins in the presence of the compound is at least 1%, at least 5%, at least 10% relative to the change observed in the absence of the compound. In certain instances of the above aspects, the change in movement is detected as an increase in immobile trajectories indicating an increase in bound (f_{bound}) target fluorescent protein. In certain instances of the above aspects, the change in movement detected is a change in: (a) the median of the jump length distribution; (b) 3^{rd} quartile of the jump length distribution; (c) median radius of gyration; (d) mean posterior diffusion coefficient; (e) geometric mean posterior diffusion coefficient; (f) mean squared displacement; (g) median bond angle; (h) diffusion coefficient maximum likelihood estimator; and/or (i) state occupation via inference. In certain instances of the above aspects, the subset of the target fluorescent proteins are in up to about 80 live cells illuminated in the field of view of the sample plane. In certain instances of the above aspects, the target fluorescent protein interacts in a larger molecular assembly. In certain instances of the above aspects, the target fluorescent protein is a ligand. In certain instances of the above aspects, the target fluorescent protein is a receptor. In certain instances of the above aspects, the biological interaction is a direct interaction. In certain instances of the above aspects, the biological interaction is an indirect interaction. In certain instances of the above aspects, the indirect interaction comprises the compound agonizing or antagonizing a larger molecular assembly comprising the target fluorescent protein.

In an interrelated aspect, the present disclosure is directed to a method of determining a dose response of a compound that induces a change in the movement of a target fluorescent protein in a live cell comprising: (a) contacting a plurality of samples with the compound, (i) wherein each sample comprises a population of live cells, (ii) wherein the live cells comprise target fluorescent protein, and (iii) wherein the plurality of samples are contacted with distinct concentrations of the compound across a range of compound concentrations; and (b) tracking the movement of individual target fluorescent proteins in a plurality of live cells of a sample, wherein said tracking comprises: (i) illuminating a field of view in a sample plane disposed within the sample with a light beam to cause fluorescence by at least a subset of the target fluorescent proteins in the live cells; (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and (c) determining a change in the movement of a target fluorescent protein in the presence of the compound; and (d) repeating steps (b)-(c) for each of the plurality of samples across the range of compound concentrations; wherein a change in the movement of the target fluorescent protein in the presence of the compound across the concentration range indicates the dose response of the compound.

In an interrelated aspect, the present disclosure is directed to a method of determining a dose response of a compound that induces a change in the movement of a target fluorescent protein in a live cell comprising: (a) contacting a plurality of samples with the compound, (i) wherein each sample comprises a population of live cells, (ii) wherein the live cells comprise the target fluorescent protein, and (iii) wherein the plurality of samples are contacted with distinct concentrations of the compound across a range of compound concentrations; (b) tracking the movement of individual target fluorescent proteins in a plurality of live cells of a sample, wherein said tracking comprises: (i) illuminating a field of view in a sample plane disposed within the sample with a light beam to cause fluorescence by at least a subset of the target fluorescent proteins in the live cells, wherein the subset of the target fluorescent proteins produces up to about 1,000,000 molecular trajectories in a single detected field of view; (ii) detecting the fluorescence from one or more of the target fluorescent proteins in the detected field of view of the sample plane via a detector device, wherein the detected field of view has a size of about 150 μm to about 250 μm in a first dimension by about 100 μm to about 210 μm in a second dimension; and (c) determining a change in the movement of the target fluorescent protein in the presence of the compound; (d) repeating steps (b)-(c) for each of the plurality of samples across the range of compound concentrations; wherein a change in the movement of the target fluorescent protein in the presence of the compound across the concentration range indicates the dose response of the compound.

In an interrelated aspect, the present disclosure is directed to a method of determining a dose response of a compound that induces a change in the movement of a target fluorescent protein in a live cell comprising: (a) contacting a plurality of samples with the compound, (i) wherein each sample comprises a population of live cells, (ii) wherein the live cells comprise the target fluorescent protein, and (iii) wherein the plurality of samples are contacted with distinct concentrations of the compound across a range of compound concentrations; (b) tracking the movement of individual target fluorescent proteins in a plurality of live cells of a sample, wherein said tracking comprises: (i) illuminating a field of view in a sample plane disposed within the sample with a light beam to cause fluorescence by at least a subset of the target fluorescent proteins in the live cells, wherein the field of view is illuminated using a 0.1 to 1 msec stroboscopic laser pulse; and (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and (c) determining a change in the movement of a target fluorescent protein in the presence of the compound; and (d) repeating steps (b)-(c) for each of the plurality of samples across the range of compound concentrations; wherein a change in the movement of the target fluorescent protein in the presence of the compound across the concentration range indicates the dose response of the compound.

In an interrelated aspect, the present disclosure is directed to a method of determining a dose response of a compound that induces a change in the movement of a target fluorescent protein in a live cell comprising: (a) contacting a plurality of samples with the compound, (i) wherein each sample comprises a population of live cells, (ii) wherein the live cells comprise the target fluorescent protein, and (iii) wherein the plurality of samples are contacted with distinct concentrations of the compound across a range of compound concentrations; and (b) tracking the movement of individual target fluorescent proteins in a plurality of live cells of a sample, wherein said tracking comprises: (i) illuminating a field of view in a sample plane disposed within the sample with a light beam to cause fluorescence by at least a subset of the target fluorescent proteins in the cells; (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and (iii) detecting the fluorescence from a plurality of the target fluorescent proteins the detected field of view of the sample plane at a rate of more than about 100,000 detected field of views per day; and (c) determining a change in the movement of a target fluorescent protein in the presence of the compound; (d) repeating steps (b)-(c) for each of the plurality of samples across the range of compound concentrations; wherein a change in the movement of the target fluorescent protein in the presence of the compound across the concentration range indicates the dose response of the compound.

In an interrelated aspect, the present disclosure is directed to a method of determining a dose response of a compound that induces a change in the movement of a target fluorescent protein in a live cell comprising: (a) contacting a plurality of samples with the compound, (i) wherein each sample comprises a population of live cells; (ii) wherein the live cells comprise the target fluorescent protein; and (iii) wherein the plurality of samples are contacted with distinct concentrations of the compound across a range of compound concentrations; (b) tracking the movement of individual target fluorescent proteins in a plurality of live cells of a sample, wherein said tracking comprises: (i) illuminating a field of view in a sample plane disposed within the sample with a light beam to cause fluorescence by at least a subset of the target fluorescent proteins in the live cells; (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and (iii) achieving, based on a single field of view, a z-factor of >0.5; and (c) determining a change in the movement of a target fluorescent protein in the presence of the compound; and (d) repeating steps (b)-(c) for each of the plurality of samples across the range of compound concentrations, wherein a change in the movement of the target fluorescent protein in the presence of the compound across the concentration range indicates the dose response of the compound.

In an interrelated aspect, the present disclosure is directed to a method of determining a dose response of a compound that induces a change in the movement of a target fluorescent protein in a live cell comprising: (a) contacting a plurality of samples with the compound, (i) wherein each sample comprises a population of live cells; (ii) wherein the live cells comprise the target fluorescent protein; and (iii) wherein the plurality of samples are contacted with distinct concentrations of the compound across a range of compound concentrations; (b) tracking the movement of individual target fluorescent proteins in a plurality of live cells of a sample, wherein said tracking comprises: (i) illuminating a field of view in a sample plane disposed within the sample with a light beam to cause fluorescence by at least a subset of the target fluorescent proteins in the live cells; and (ii) detecting the fluorescence from one or more of the target fluorescent proteins in the sample plane via a detector device, wherein the field of view is detected with a frame rate up to about 2000 Hz and wherein the detected field of view has a size of about 150 μm to about 250 μm in a first dimension by about 100 μm to about 210 μm in a second dimension; and (c) determining a change in the movement of a target fluorescent protein in the presence of the compound; and (d) repeating steps (b)-(c) for each of the plurality of samples across the range of compound concentrations, wherein a change in the movement of the target fluorescent protein in the presence of the compound across the concentration range indicates the dose response of the compound.

In an interrelated aspect, the present disclosure is directed to a method of determining a dose response of a compound that induces a change in the movement of a target fluorescent protein in a live cell comprising: (a) contacting a plurality of samples with the compound, (i) wherein each sample comprises a population of live cells; (ii) wherein the live cells comprise the target fluorescent protein; and (iii) wherein the plurality of samples are contacted with distinct concentrations of the compound across a range of compound concentrations; (b) tracking the movement of individual target fluorescent proteins in a plurality of live cells of a sample, wherein said tracking comprises: (i) illuminating a field of view in a sample plane disposed within the sample with a light beam to cause fluorescence by at least a subset of the target fluorescent proteins in the live cells; and (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension and wherein equal to or greater than 95% of the detected field of view achieves sufficient laser illumination for tracking protein movement; and (c) determining a change in the movement of a target fluorescent protein in the presence of the compound; and (d) repeating steps (b)-(c) for each of the plurality of samples across the range of compound concentrations, wherein a change in the movement of the target fluorescent protein in the presence of the compound across the concentration range indicates the dose response of the compound.

In certain instances of the above aspects, the average change in movement of the target fluorescent proteins in the presence of the compound is at least 1%, at least 5%, at least 10% relative to the change observed in the absence of the compound. In certain instances of the above aspects, the change in movement detected is an increase in immobile trajectories indicating an increase in bound (f_{bound}) target fluorescent protein. In certain instances of the above aspects, the movement detected is a change in: (a) the median of the jump length distribution; (b) 3^{rd} quartile of the jump length distribution; (c) median radius of gyration; (d) mean posterior diffusion coefficient; (e) geometric mean posterior diffusion coefficient; (f) mean squared displacement; (g) median bond angle; (h) diffusion coefficient maximum likelihood estimator; and/or (i) state occupation via inference. In certain instances of the above aspects, the subset of the target fluorescent proteins are in up to about 80 live cells illuminated in the field of view of the sample plane. In certain instances of the above aspects, the target fluorescent protein interacts in a larger molecular assembly. In certain instances of the above aspects, the target fluorescent protein is a ligand. In certain instances of the above aspects, the target fluorescent protein is a receptor. In certain instances of the above aspects, the biological interaction is a direct interaction. In certain instances of the above aspects, the direct interaction comprises binding of the compound to the target fluorescent protein. In certain instances of the above aspects, the biological interaction is an indirect interaction. In certain instances of the above aspects, the indirect interaction comprises the compound agonizing or antagonizing a larger molecular assembly comprising the target fluorescent protein.

In an interrelated aspect, the present disclosure is directed to a microscopy system configured to identify a biological interaction between a compound and a target fluorescent protein in a live cell comprising: (a) a stage for supporting a sample, wherein the sample comprises a population of live cells, and wherein the live cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a subset of the target fluorescent proteins in the sample are disposed in a detected field of view of the sample plane and wherein the detected field of view has a size of about 150 μm to about 250 μm in a first dimension by about 100 μm to about 210 μm in a second dimension; (d) a detector device for monitoring the light-based response from the target fluorescent proteins in the presence of the compound; (e) a memory; and (f) a processor in communication with the memory and the detector device, where the processor is capable of determining the change in the movement of the target fluorescent protein in the presence of the compound relative to the absence of the compound.

In an interrelated aspect, the present disclosure is directed to a microscopy system configured to identify a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a subset of the target fluorescent proteins in the sample are disposed in a detected field of view of the sample plane and wherein the subset of the target fluorescent proteins produces up to about 1,000,000 molecular trajectories in a single field of view and wherein the detected field of view has a size of about 150 μm to about 250 μm in a first dimension by about 100 μm to about 210 μm in a second dimension; (d) a detector device for monitoring the light-based response from the target fluorescent proteins in the presence of the compound; (e) a memory; and (f) a processor in communication with the memory and the detector device, where the processor is capable of determining the change in the movement of the target fluorescent protein in the presence of the compound relative to the absence of the compound.

In an interrelated aspect, the present disclosure is directed to a microscopy system configured to identify a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample, wherein a field of view is illuminated using a 0.1 to 1 msec stroboscopic laser pulse; (c) an objective for focusing the light beam on the sample in the sample plane, wherein a subset of the target fluorescent proteins in the sample are disposed in a detected field of view of the sample plane and wherein the detected field of view has a size of about 150 μm to about 250 μm in a first dimension by about 100 μm to about 210 μm in a second dimension; (d) a detector device for monitoring the light-based response from the target fluorescent proteins in the presence of the compound; (e) a memory; and (f) a processor in communication with the memory and the detector device, where the processor is capable of determining the change in the movement of the target fluorescent protein in the presence of the compound relative to the absence of the compound.

In an interrelated aspect, the present disclosure is directed to a microscopy system configured to identify a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a plurality of the target fluorescent proteins in the sample are disposed in a detected field of view of the sample plane and wherein the detected field of view has a size of about 150 μm to about 250 μm in a first dimension by about 100 μm to about 210 μm in a second dimension; (d) a detector device for monitoring the light-based response from the target fluorescent proteins in the presence of the compound, wherein said monitoring comprises detecting the fluorescence from a plurality of the target fluorescent proteins in the detected field of view of the sample plane at a rate of more than about 100,000 detected field of views per day; (e) a memory; and (f) a processor in communication with the memory and the detector device, where the processor is capable of determining the change in the movement of the target fluorescent protein in the presence of the compound.

In an interrelated aspect, the present disclosure is directed to a microscopy system configured to identify a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a plurality of the target fluorescent proteins in the sample are disposed in a detected field of view of the sample plane and wherein the detected field of view has a size of about 150 μm to about 250 μm in a first dimension by about 100 μm to about 210 μm in a second dimension; (d) a detector device for monitoring the light-based response from the target fluorescent proteins in the presence of the compound; (e) a memory; and (f) a processor in communication with the memory and the detector device, wherein the processor is capable of determining the change in the movement of the target fluorescent protein in the presence of the compound, wherein the system has a z-factor of >0.5 based on a single detected field of view.

In an interrelated aspect, the present disclosure is directed to a microscopy system configured to identify a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a plurality of the target fluorescent proteins in the sample are disposed in a detected field of view of the sample plane and wherein the detected field of view has a size of about 150 μm to about 250 μm in a first dimension by about 100 μm to about 210 μm in a second dimension; (d) a detector device for monitoring the light-based response from the target fluorescent proteins in the presence of the compound, wherein the detector device has a frame rate up to about 2000 Hz; (e) a memory; and (f) a processor in communication with the memory and the detector device, wherein the processor is capable of determining the change in the movement of the target fluorescent protein in the presence of the compound.

In an interrelated aspect, the present disclosure is directed to a microscopy system configured to identify a biological interaction between a compound and a target fluorescent protein in a live cell comprising: (a) a stage for supporting a sample, wherein the sample comprises a population of cells, and where the cells comprise the target fluorescent protein; (b) a light source for emitting a light beam capable of inducing a light-based response from a plurality of the target fluorescent proteins in the sample; (c) an objective for focusing the light beam on the sample in the sample plane, wherein a plurality of the target fluorescent proteins in the sample are disposed in a detected field of view of the sample plane, wherein the detected field of view has a size of about 150 μm to about 250 μm in a first dimension by about 100 μm to about 210 μm in a second dimension and wherein equal to or greater than about 95% of the detected field of view achieves sufficient laser illumination for tracking protein movement; (d) a detector device for monitoring the light-based response from the target fluorescent proteins in the presence of the compound; (e) a memory; and (f) a processor in communication with the memory and the detector device, wherein the processor is capable of determining the change in the movement of the target fluorescent protein in the presence of the compound.

In certain instances of the above aspects, the average change in movement of the target fluorescent proteins in the presence of the compound is at least 1%, at least 5%, at least 10% relative to the change observed in the absence of the compound. In certain instances of the above aspects, the change in movement detected is an increase in immobile trajectories indicating an increase in bound (f_{bound}) target fluorescent protein. In certain instances of the above aspects, the movement detected is a change in: (a) the median of the jump length distribution; (b) 3^{rd} quartile of the jump length distribution; (c) median radius of gyration; (d) mean posterior diffusion coefficient; (e) geometric mean posterior diffusion coefficient; (f) mean squared displacement; (g) median bond angle; (h) diffusion coefficient maximum likelihood estimator; and/or (i) state occupation via inference. In certain instances of the above aspects, the subset of the target fluorescent proteins are in up to about 80 live cells illuminated in the field of view of the sample plane. In certain instances of the above aspects, the target fluorescent protein interacts in a larger molecular assembly. In certain instances of the above aspects, the target fluorescent protein is a ligand. In certain instances of the above aspects, the target fluorescent protein is a receptor. In certain instances of the above aspects, the biological interaction is a direct interaction. In certain instances of the above aspects, the direct interaction comprises binding of the compound to the target fluorescent protein. In certain instances of the above aspects, the biological interaction is an indirect interaction. In certain instances of the above aspects, the indirect interaction comprises the compound agonizing or antagonizing a larger molecular assembly comprising the target fluorescent protein.

### BRIEF DESCRIPTION OF THE DRAWINGS

The patent or application file includes at least one drawing executed in color. Copies of this patent or patent application publication with color drawing(s) will be provided by the Office upon request and payment of the necessary fee.

FIG. 1 depicts a schematic of the htSMT workflow.

FIGS. 2A-2F depict an exemplary image acquisition system of the present disclosure with the X-Z sample plane visible (FIGS. 2A and 2D) or the Y-Z sample plane visible (FIGS. 2B and 2C), as well as detail regarding the light beam relative to a HILO-based approach (FIG. 2E, OLS on the left and HILO on the right) and an example of the incorporation of a camera rolling shutter (FIG. 2F).

FIG. 3A-3E depict various measures indicating that the image acquisition systems and workflows of the present disclosure are amenable to robust htSMT analysis. FIG. 3A depicts a laser titration experiment indicating the relationship of laser power at the sample (mW) to signal-to-noise ratio (SNR) (left panel) as well as the average SNR at the well-level across four image acquisition systems measuring six different 384 well plates per system (right panel). FIG. 3B depicts differences in the heterogeneity in spatial SNR between the OLS systems of the instant disclosure and HILO-based approaches. The top panel compares the spatial standard deviations observed in OLS relative to a HILO-based approach. The bottom panel illustrates the difference in FOV between HILO and OLS-based approaches (left image), along with a comparison of the spatial heterogeneity across those FOVs for each of the HILO-based approach (middle image) and the OLS-based approach (right image). FIG. 3C depicts a dose-response experiment conducted on a Halo-tagged protein with an established and well-characterized compound to assess plate to plate and day to day reproducibility (top panels) and the respective EC50s presented (bottom panel). FIG. 3D indicates that the systems described herein are configured to capture comparable protein diffusion coefficients per FOV per well, where each point represents individual FOV positions averaged per plot for each concentration (top panel) and both the EC50s and Z-Factors are presented (bottom panel). FIG. 3E depicts the consistency of data across multiple wells and multiple experiments, where each point represents one FOV from 14 independently generated dose-response curves.

FIG. 4 depicts a comparison of Z-factors associated with data presented in FIG. 3D and FIG. 3E to data collected using a HILO-based approach.

FIG. 5 depicts a schematic of an exemplary sample handling system of the present disclosure.

FIG. 6 illustrates an exemplary system for high-throughput single-molecule imaging platform that measures protein movement in living cells.

FIG. 7 illustrates data flow through an exemplary system for a high-throughput single-molecule imaging platform that measures protein movement in living cells.

FIG. 8 depicts a plurality of images illustrating differences between mask categories and instance or semantic masks.

FIG. 9 illustrates an example computer-implemented environment in connection with the subject matter described herein.

FIG. 10 is a diagram illustrating a sample computing device architecture for implementing various aspects described herein.

FIGS. 11A-11G illustrate that OLS provides near full field homogeneous illumination enabling expansive SMT. FIG. 11A depicts a simplified schematic describing the OLS implementation. Briefly, a collimated beam is shaped into an optical light-sheet, which is sent to a water-immersion objective with the emission being projected onto a high-speed sCMOS camera. FIG. 11B depicts an exemplary SMT workflow relying on the Halo-tagging of protein targets of interest. JF₅₄₉ or JF₆₄₆ organic fluorophores were used to detect individual emitters with appropriate signal to conduct frame to frame linking and track generation. From these coordinates and trajectories, a variety of metrics can be extracted including protein diffusion and spatial localization amongst others. FIG. 11C depicts a 20-point dose-response curve from 6-7 distinct 384-well plates per microscope imaged on Eikon's high throughput SMT platform. 72 FOVs from 12 wells were captured for each concentration on randomized plates, error bars denote standard deviation. FIG. 11D depicts representative sampling areas in HILO and OLS for illumination from Halo-Keap1 containing U2OS cells. Trajectories were plotted across a 1.5 s acquisition and color coded based on the measured diffusion coefficient with nuclear mask outlines overlaid with a black dotted line. FIG. 11E depicts the quantification of the number of trajectories captured per FOV using HILO and OLS, where OLS captures a 6-fold improvement. FIG. 11F depicts representative average spatial SNR maps per pixel calculated across 1,232 FOVs for a plate imaged with HILO or OLS. OLS provides a 6× larger FOV along with an improvement in illumination homogeneity. FIG. 11G provides the average FOV-level standard deviation in SNR sampled over 308 wells.

FIGS. 12A-12G depict an exemplary schematic of an OLS microscope for single-molecule tracking. FIG. 12A depicts an exemplary schematic of an OLS microscope. FIG. 12B depicts an exemplary schematic of an OLS microscope based on scanning an inclined excitation light sheet using galvanometric scanning mirrors across a sample placed in an inverted microscope. The OLS microscope is based on a multiwavelength optical excitation provided by a Laser Engine Module (LEM) and coupled to the beam shaper by a collimator-coupled single-mode fiber. The beam shaper transforms the incoming Gaussian-shaped optical excitation to an optical light-sheet that is focused into the back focal plane of the microscope's objective lens along the light sheet's line axis and scanned along the scan axis using galvanometric mirrors. The resulting oblique light sheet is sent into a water-immersion-coupled and environmentally-controlled sample holding plate, whereas the relative position of the microscope's focal plane is controlled by an autofocus unit. The excited fluorescence is spectrally filtered from the excitation light by dichroic filters and emission filters and projected onto a high-speed sCMOS camera. Synchronization of optical excitation, scanning, and acquisition is achieved by a custom-build control unit (MIC). FIG. 12C depicts an exemplary schematic of an autofocus unit which is based on detecting a 780 nm-LED reflection on the top surface of the sample-holding glass bottom and repositioning the objective lens to ensures appropriate focal plane positioning within the sample. FIG. 12D depicts an exemplary schematic of an optical confocal scanning mode achieved by scanning an inclined and focused light sheet through the objective's focal plane. Background suppression is achieved by confocal arrangement of the inclined light sheet (green), the objective's depth of field, and synchronized rolling shutter detection (orange). FIG. 12E depicts an exemplary schematic of a beam shaping subassembly that is projected along the line axis (x) and the scan axis (y) shapes collimated optical excitation into a light sheet by a series consisting of a Powell lens, cylindrical lenses, a spherical lens, and a planoconvex lens before encountering the galvanometric scanning mirror. Insets depict optical beam profile at the respective positions. FIG. 12F depicts an exemplary schematic of an optical line scanning framework based on an inclined light sheet in the sample plane achieved by focusing the optical excitation along the line axis in the objective's back focal plane and positioning the optical excitation along the scan axis at an offset position relative to the optical axis of the objective. The corresponding detection of optically-aligned fluorescence is projected onto the camera sensor. FIG. 12G depicts an exemplary schematic of an OLS acquisition mode that relies on detecting fluorescence by matching the camera's area of exposed pixels and synchronizing the camera's rolling shutter to the optically-projected intensity line of fluorescence excited by the inclined light sheet.

FIGS. 13A-13F depict the characterization of motion-induced blurring and confocality between OLS and HILO illumination. FIG. 13A depicts a bar graph comparing measured diffusion coefficient for Halo-KEAP1 treated with DMSO or 1 mM KI-696 across 72 FOVs from 12 individual wells for HILO and OLS. Despite the extensive sampling, standard deviation in the measurement remains larger for HILO. FIG. 13B depicts the estimated point spread functions, found by averaging all detections in a representative 150-frame acquisition. The following number of PSFs were detected for each condition: n=123,596 (OLS-DMSO), n=3,897 (HILO-DMSO), n=113,276 (OLS 0.33 mM KI-696), n=13,620 (HILO 0.33 mM KI-696) from one representative FOV. FIG. 13C depicts the PSF detections as a function of integration time. HILO required 5× longer integration time to achieve comparable PSF detection and spot density to OLS which resulted in a more pronounced motion-induced blurring in HILO. FIG. 13D depicts the PSF width measurement as a function of JF₅₄₉ measured in Halo-KEAP1 cells treated with 1 mM KI-696. FIG. 13E depicts the mean SNR plotted as a function of JF₅₄₉ measured in Halo-KEAP1 cells treated with 1 mM KI-696. FIG. 13F illustrates the number of spot detections plotted as a function of JF₅₄₉ measured in increasing concentrations of Halo-JF₅₄₉ in solution.

FIGS. 14A-14D illustrate that OLS enables reproducible and robust SMT measurements. FIG. 14A depicts EC₅₀ values calculated from each averaged dose-response curve per plate per microscope, black lines represent the median EC₅₀. FIG. 14B depicts a violin plot of the signal to noise ratio (SNR) per microscope, heavy dashed line represents median value. FIG. 14C depicts a violin plot of the SNR as a function of FOV position within an acquired well. FIG. 14D depicts a 20-point dose-response curve for Halo-KEAP1 U2OS sampled at full OLS FOV (purple) versus a cropped FOV of 768×768 pixels (black) representative of a HILO-sized FOV. Error bars denote standard deviation between FOVs.

FIGS. 15A-15C illustrate that OLS enables the capture of fast protein diffusion in living cells. FIG. 15A depicts representative images of FOV size for each of five frame rates ranging from 100-1250 Hz. Trajectories are overlaid onto a mean projection for the Hoechst channel (blue), and colored by their maximum likelihood diffusion coefficients. FIG. 15B depicts the fraction of trajectories with a diffusion coefficient >10 μm²/s as a function of frame rate for each of DMSO and KI-696-treated cells, computed from the state array posterior mean occupations. FIG. 15C depicts the accuracy of state profile recovery from optical-dynamical simulations of SMT across several frame rates for 3 distinct state mixtures. Error bars denote S.D.

FIGS. 16A-16E illustrate that frame rate determines SMT dynamic range. FIG. 16A depicts a schematic describing the role of localization and tracking errors for a hypothetical fast moving protein. The rolling shutter in OLS captures the position of dye molecules at discrete timepoints. If these timepoints are too close, the apparent motion is dominated by localization error. If the timepoints are too far apart, reconstructing trajectories becomes challenging and is dominated by misconnections. FIG. 16B depicts a schematic describing the dynamic range of SMT, bounded on one end by localization error and on the other by tracking errors. An approximation of this range for Brownian motion is σ_{loc}²/Δt≤\hat{D}≤R²/8Δt where σ² is the localization error variance, Δt is the frame interval, R is the search radius, and D is the diffusion coefficient. FIG. 16C depicts a schematic of a simulation approach to test the role of frame rate. Movies were simulated with real-world effects including defocus, motion blur, shot noise, and read noise. FIG. 16D depicts the effect of frame rate has on linking precision and track length. Linking precision is defined as the fraction of links made by the tracking algo that are correct; track length is the number of points in each trajectory. Quantiles are over simulated movies. FIG. 16E depicts the state array posterior mean occupations for three simulated dynamical mixtures at increasing frame rate. Red lines correspond to the simulated discrete mixture model, blue lines to the state array posterior means, and green lines to the expected SMT dynamic range as defined in (B). Ten simulation replicates were included for each condition.

FIGS. 17A-17C depict the tracking diagnostics for experimental KEAP1-HaloTag JF549 SMT in U2OS cells with varying frame rates. FIG. 17A depicts the mean trajectory length plotted as a function of frame rate. Trajectory length is defined as the number of spots per trajectory. FIG. 17B depicts the mean SNR plotted as a function of frame rate. SNR is described in Example 2. FIG. 17C depicts mean ERLB plotted as a function of frame rate.

FIG. 18 provides the state array analysis plotted as a function of frame rate comparing DMSO and 1 mM KI-696 treated Keap1-HaloTag U2OS cells. The number of FOV replicates per frame rate was as follows: n=88 (100 Hz), n=88 (200 Hz), n=132 (400 Hz), n=198 (800 Hz), and n=264 (1250 Hz). Lines are the mean values over all FOVs in the corresponding condition, and error bands are the FOV-level standard deviations.

FIG. 19 provides the evaluation of bleaching rate in KEAP1-HaloTag SMT at variable frame rates. Fraction detections remaining were plotted across frame rate for a given time series. The fraction of detections remaining was defined as the number of detections in each frame divided by the number of detections in the first frame. Exponential fits (blue text below frame rate) were performed with respect to the model f(t)=c₀+(1−c₀)e^{-kt}, where t is frame index, k is bleaching rate, and c₀ is the unbleached fraction, using an iterative least-squares routine. The number of FOV replicates per frame rate was as follows: n=88 (100 Hz), n=88 (200 Hz), n=132 (400 Hz), n=198 (800 Hz), and n=264 (1250 Hz).

FIGS. 20A-20F illustrate that OLS can be applied to capture the inter and intracellular heterogeneity of single protein dynamics. FIG. 20A depicts the analysis of sources of variance in SMT for KEAP1 measured under either OLS or HILO illumination. The contribution of cell to cell variation is 17-32 fold greater than that of FOV level or well to well variances, respectively. FIG. 20B depicts representative images of Halo-PCNA labeled cells treated with 2 mM Thymidine or 10 mM RO-3306 (top). Cell cycle prediction from a machine learning (ML) model used to color cell by cycle phase (bottom). FIG. 20C depicts the quantification of the fraction of cells in each cell phase in response to cycle block treatments in FIG. 20B. The following number of cells were analyzed for each condition: 34,067 for DMSO, 3,831 for RO-3306 and 6,044 for thymidine. FIG. 20D depicts a state array analysis of total population of sparsely labeled PCNA cells. FIG. 20E depicts a state array analysis for cells in each phase as predicted by the ML model. FIG. 20F depicts a heat map of 4,801 individual cells classified using a continuous classification score plotted against PCNA diffusion coefficient.

FIGS. 21A-21D illustrate the characterization of the PCNA-based cell cycle prediction model. FIG. 21A provides example images from time-lapse of PCNA captured on OLS with 5 min frame interval for 12 hours. FIG. 21B depicts a schematic of a neural network trained to simultaneously perform segmentation of nuclei, cell cycle classification of nuclei, and cell cycle regression of nuclei. FIG. 21C depicts a confusion Matrix describes the performance of the cell cycle classification. FIG. 21D provides a representative image of cell cycle progression over a 12-hour window for 4 selected cells (left) with a graph of regression-based prediction of cell cycle progression plotted with a 5 frame moving average (right).

FIGS. 22A-22D depict PCNA cell line validation using Western blot and cell proliferation analysis. FIG. 22A depicts a capillary-based Western blot comparing WT U2OS and N-terminal tagged heterozygous PCNA clone with anti-PCNA antibody (left) and anti-Halo antibody (right). FIG. 22B depicts relative WT and Halo-tagged PCNA levels normalized to β-actin WT and Halo-edited U2OS cells. FIG. 22C depicts a growth curve of WT U2OS and N-terminal Halo-Tagged PCNA. FIG. 22D depicts cells labeled with both JF₅₄₉ and CCR PCNA to measure spatial colocalization between the two labels over the course of the cell cycle.

FIGS. 23A-23M illustrate that OLS is amenable to a variety of SMLM techniques and acquisition schemes. FIG. 23A depicts JF₅₄₉ and JF₆₄₆ labeled Halo-KEAP1 U2OS cells imaged within the same FOV. FIG. 23B depicts a 10-point dose-response of KI-696-treated Halo-KEAP1 U2OS cells co-labeled with JF₅₄₉ and JF₆₄₆. FIG. 23C depicts a diffraction-limited image of a full OLS FOV of immunofluorescently labeled tubulin with AF647-conjugated secondary antibody. FIG. 23D depicts a zoomed-in region of interest from FIG. 23C. FIG. 23E depicts a STORM reconstruction of the full OLS FOV, labeled as in FIG. 23C. FIG. 23F depicts a zoomed-in region of interest from FIG. 23E as in FIG. 23D. FIG. 23G depicts a line profile from yellow lines in FIG. 23D and FIG. 23F gray value (a.u) to compare spatial resolution of microtubules. FIG. 23H depicts the localization precision histogram for each of AF647 and CF568 labeled secondary antibodies used to stain microtubules with OLS illumination with a 0.4 msec integration time. FIG. 23I depicts a representative image of correlative FRAP/SMT where a central region was bleached using the OLS line scan prior to spot recovery after photobleaching. Areas outside and inside of the FRAP region were used to measure SMT. FIG. 23J depicts the T_{1/2} FRAP for Halo-KEAP1 U2OS cells treated with DMSO or 1 mM KI-696 labeled with 400 pM JF₅₄₉-Halo ligand, black line denotes median, and each spot represents an individual FOV. FIG. 23K and FIG. 23L depict the spot density after recovery over time for each of DMSO (FIG. 23K) and 1 mM KI-696 (FIG. 23L). Standard deviation shown in confidence bands for 8-10 FOVs per condition. FIG. 23M depicts the diffusion coefficient from SMT for 400 pM JF₅₄₉-Halo ligand concentration in bleached (Inside) and unbleached (Outside) region.

FIGS. 24A-24C illustrate the characterization of dye performance and FRAP with increasing dye concentration. FIG. 24A depicts the SNR comparison between JF₆₄₆ and JF₅₄₉. FIG. 24B depicts the ERLB comparison between JF₆₄₆ and JF₅₄₉. FIG. 24C depicts the sampling of T_{1/2} measured in the bleached region as a function of dye concentration for 6-10 FOVs for DMSO and 1 mM KI-696.

FIGS. 25A-25B illustrate the contribution of well-to-well, FOV-to-FOV, and cell-to-cell biases to 2D jump length, assessed using jump resampling. FIG. 25A depicts the variance over sample means as a function of sample size for different resampling procedures. A straight line with slope-1 is the expectation from the law of large numbers; sublinearities are due to residual variances over wells, FOVs, or cells. FIG. 25B depicts the number of jumps per well, FOV, or cell used in these analyses.

### DETAILED DESCRIPTION

The presently disclosed subject matter relates to the development of industrial-scale, high-throughput SMT (htSMT) techniques employing oblique line scanning (OLS) illumination, systems incorporating such OLS htSMT techniques, hardware and software related to such OLS htSMT techniques, as well as methods of using such OLS htSMT techniques. For example, the OLS htSMT techniques described herein are capable of measuring protein movement in millions of cells per day. In addition to the ability to capture a large number of cells per field of view, OLS benefits from an improved spatial homogeneity in signal to noise ratio (SNR) across the camera chip, better confocality (less out of focus signal and reduced motion blurring) and higher temporal resolution, e.g., as outlined in Table 1 (where each “+” represents a 2-fold improvement).

TABLE 1

| Parameter | OLS | HILO |
|---|---|---|
| Spatial SNR Homogeneity | +++ | + |
| Confocality | +++ | + |
| Temporal Resolution | +++ | + |
| FOV Size | ++++ | + |

The OLS htSMT techniques described herein can be used for a variety of applications including, but not limited to, drug discovery activities, such as compound library screening and the elucidation of structure-activity relationships (SAR). Importantly, the OLS htSMT techniques described herein can be used to characterize both known and novel pathway contributions to larger molecular assemblies comprising the target, such as protein signaling interaction networks.

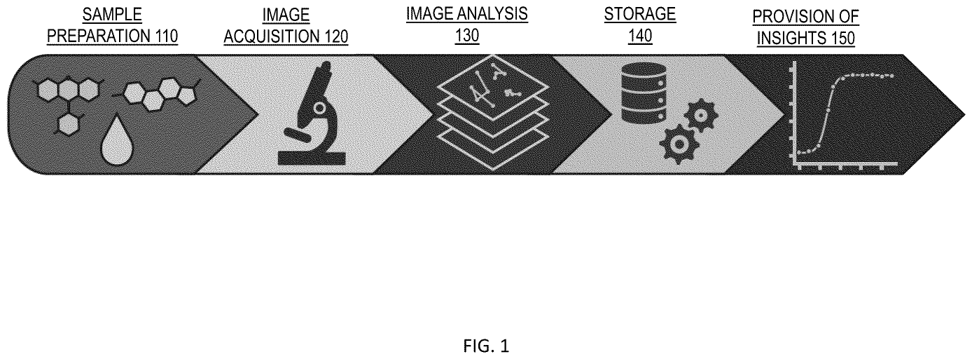

With reference to FIG. 1, aspects of the current subject matter can be implemented using an OLS htSMT workflow. This workflow can include various phases, as will be described in further detail below, such as (i) sample preparation including reagent handling, (ii) image acquisition using imaging of the samples to generate a series of images and/or videos, (iii) image analysis through processing of these images and video using, for example, various analytics, single-emitter detection and sub-pixel localization (i.e., “super resolution imaging”), tracking, computer vision, and machine learning algorithms, (iv) storage of information (i.e., features, raw images, modified images, etc.) extracted from or otherwise characterizing or comprising the images and video, and (v) provision of insights using the stored information including biological interpretation (which can additionally or alternatively be provided using various analytics, tracking, computer vision, and machine learning algorithms).

The subject matter of the present disclosure is described with reference to the figures, where reference numbers are used to designate similar or equivalent elements throughout. The figures are not drawn to scale and they are provided merely to illustrate aspects disclosed herein. Several disclosed aspects are described below with reference to exemplary hardware, software, and applications for illustration. It should be understood that numerous specific details, relationships and methods are set forth to provide a more complete understanding of the subject matter disclosed herein. For purposes of clarity of disclosure and not by way of limitation, the detailed description is divided into the following subsections:
1. Definitions
2. OLS htSMT Hardware
3. OLS htSMT Software
4. Specific OLS htSMT Applications
5. Exemplary Embodiments
6. Examples

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other instances “comprising,” “consisting of”, and “consisting essentially of,” the instances or elements presented herein, whether explicitly set forth or not.

For the recitation of numeric ranges herein, each intervening number within the range is explicitly contemplated with the same degree of precision. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein the term “trajectory” refers to the set of spatial coordinates corresponding to the position of an observation of fluorescent protein, linked in time. In certain instances, a plurality of trajectories may be constructed algorithmically by linking a plurality of fluorescent proteins whose positions have been determined in successive time points. In certain instances, a plurality of trajectories may be constructed conservatively by linking only spots within a fixed search radius when no other links are plausible. In certain instances, a plurality of trajectories may be constructed probabilistically.

As defined herein, protein movement refers to the change in position of a plurality of fluorescent proteins. In certain instances, protein movement may be quantified by analysis of changes in spatial coordinates in sequential timepoints. Movement characterized in this way may include, but not be limited to, measurements of the jump length distribution: Given a set of protein displacements between one timepoint and a subsequent timepoint, a histogram can be constructed of the probability of each of the displacement lengths (“jump lengths”). Quantiles of this distribution can be used to describe the motion of the protein. In certain instances the quantile used is the median of the jump length distribution. In certain instances, the quantile used is the 3^{rd} quartile of the jump length distribution. In certain instances, protein movement may be quantified by analysis of trajectories. Movement characterized in this way may include, but not be limited to, measurements of the mean squared displacement as defined by the average of the square of all displacements in a trajectory, averaged over the plurality of trajectories. Movement characterized in this way may also include, but not be limited to, measurements of the trajectory length or distribution of trajectory lengths. Movement characterized in this way may also include, but not be limited to, measurements of the mean radius of gyration, as defined by the root mean square distance of all coordinates in a trajectory from the center of mass of the set of points contained in the trajectory, averaged over the plurality of trajectories. Movement characterized in this way may also include, but not be limited to, measurements of the mean bond angle, defined by the angle formed from three sequential spatial coordinates averaged over the plurality of trajectories. Movement characterized in this way may also include, but not be limited to, measurements of the diffusion coefficient maximum likelihood estimator, defined as an estimate of the maximum likelihood diffusion coefficient for the plurality of trajectories under a single-state diffusion model with constant localization error. In certain instances, protein movement may be measured by measured through analysis of the product of the link-generating algorithm. Movement characterized in this way may include, but not be limited to, the mean posterior diffusion coefficient, the mean of the posterior probability distribution of coefficients from a probabilistic linking algorithm. Movement characterized in this way may include, but not be limited to, the geometric mean posterior diffusion coefficient, the mean of the log-scaled posterior probability distribution of coefficients from a probabilistic linking algorithm. In certain instances, protein movement may be measured by measured through model-dependent analysis of the plurality of trajectories. Movement characterized in this way may include, but not be limited to, the fraction of immobile molecules (“f_{bound}”) as defined by two-state model fitting.

As used herein, the term “movement” encompasses changes in the direction as well as changes, both increases and decreases, in the speed at which a target is traveling. Accordingly, tracking movement can, in certain instances, include determining that the target is not moving, e.g., when the target either is or is essentially in a static bound state. Movement can be characterized in a variety of ways, including, but not limited to, quantifying: (a) the median of the jump length distribution (where the jump length corresponds to the observed distance the target fluorescent protein travels in consecutive frames); (b) 3rd quartile of the jump length distribution; (c) median radius of gyration; (d) mean posterior diffusion coefficient; (e) geometric mean posterior diffusion coefficient; (f) mean squared displacement; (g) median bond angle; (h) diffusion coefficient maximum likelihood estimator; (i) trajectory length; and/or (j) state occupation via inference.

As used herein, the movement being detected, including, but not limited to, any change in movement, can occur in response to any environmental or other factor. For example, but not by way of limitation, the movement, or lack thereof, can be elicited by: (A) compound addition; (B) a change in temperature; (C) a change in oxygen concentration, e.g., introduction of a hypoxic condition; (D) mechanical stress; (E) a change in pH; and/or (F) a change in light exposure (e.g., increasing or decreasing intensity).

As used herein, the term “fluorescent protein” refers to any protein that emits a fluorescent signal. In certain instances, the fluorescent emission occurs in response to exposure to light of a particular wavelength. An example of a naturally occurring fluorescent protein is Green fluorescent protein (GFP). In certain instances, however, a protein of interest can be adapted to emit a fluorescent signal via the introduction of an encoded fluorescent tag, i.e., a protein sequence is fused to a protein of interest to render it fluorescent. In certain instances, a protein of interest can be adapted to emit a fluorescent signal through binding of a fluorescent ligand. Nonlimiting examples of such encoded fluorescent tags include: Halo tags, SNAP tags, CLIP tags, TMP tags, and SunTags. Additionally, or alternatively, a protein of interest can be adapted to emit a fluorescent signal via coupling the protein to a fluorescent dye molecule, e.g., amine- or sulfhydryl-reactive dyes.

As used herein, the term “compound” refers to any chemically-defined entity. In certain instances, the compound can be a molecule less than 1000 Da, i.e., a “small molecule”. In certain instances, the compound can be a macromolecule such as a nucleic acid. In certain instances, the nucleic acid can have a defined sequence. In certain instances, the nucleic acid comprises; (A) ribonucleic acid (RNA), including, for example, modified RNA; (B) deoxyribonucleic acid (DNA), including, for example, modified DNA; as well as (C) combinations of (A) and (B). In certain instances, the nucleic acid will be a single-stranded or double-stranded small interfering nucleic acid (e.g., a double-stranded siRNA), an antisense oligonucleotide, a ribozyme, a microRNA, or an aptamer. In certain instances, the compound can be a protein. For example, but not by way of limitation, the protein compounds of the present disclosure encompass signaling proteins, e.g., protein hormones, cytokines, kinases, phosphatases, and other enzymes and transcription factors, as well as antibodies, contractile proteins, structural proteins, storage proteins, and transport proteins. In certain instances, a compound can refer to a mixture of molecules, e.g., a mixture of defined composition.

As used herein, the term “uniform intensity” refers, in connection with the intensity of light, e.g., light directed to a sample plane, to light that differs in intensity no more than 5%, in certain instances, 10%, in certain instances, or 15%, in certain instances.

As used herein, the term “uniform intensity” refers, in connection with signal to noise (SNR), to a pixel-wise SNR within a field of view (FOV) where the range of possible values are comprised between 0.5 to 1 standard deviations from the mean SNR.

### 2. OLS HTSMT HARDWARE

### 2.1. Image Acquisition Systems

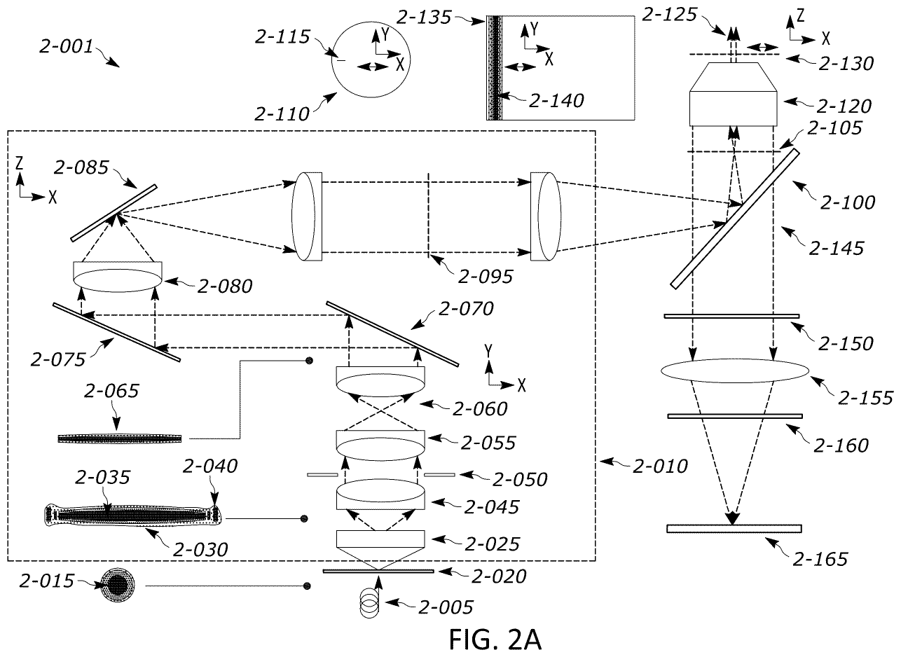

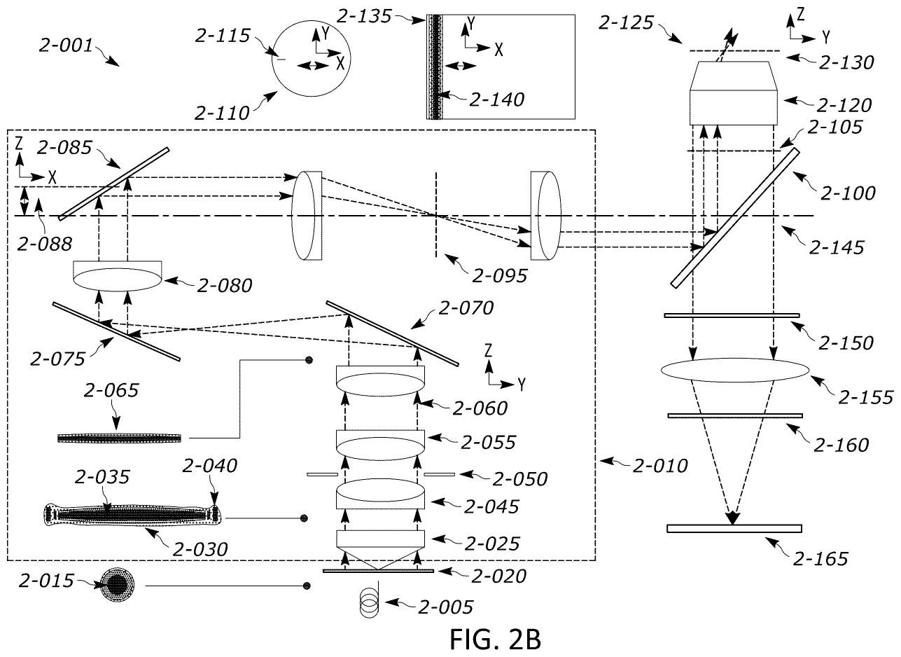

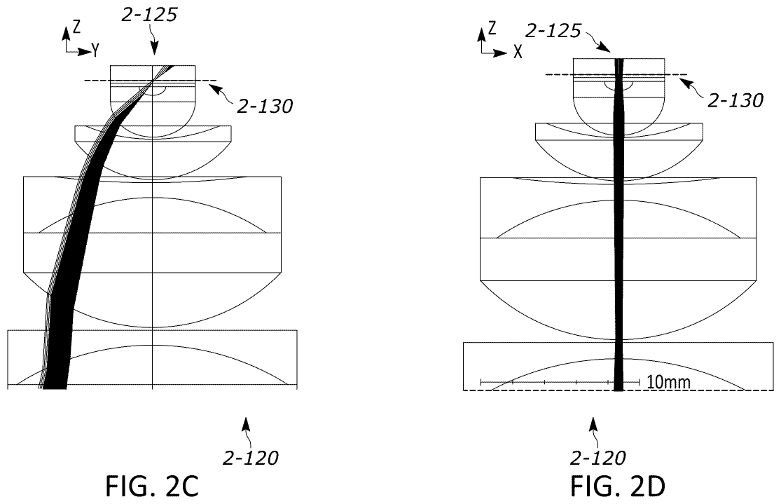

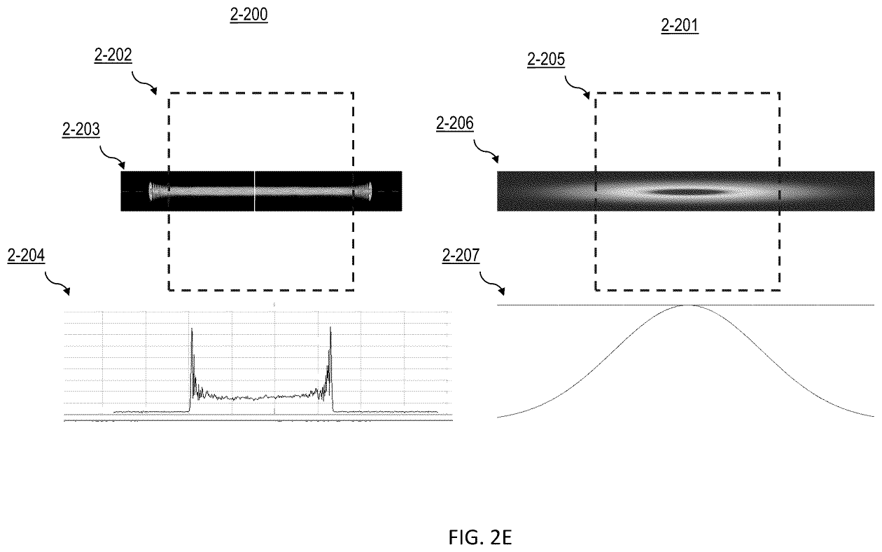

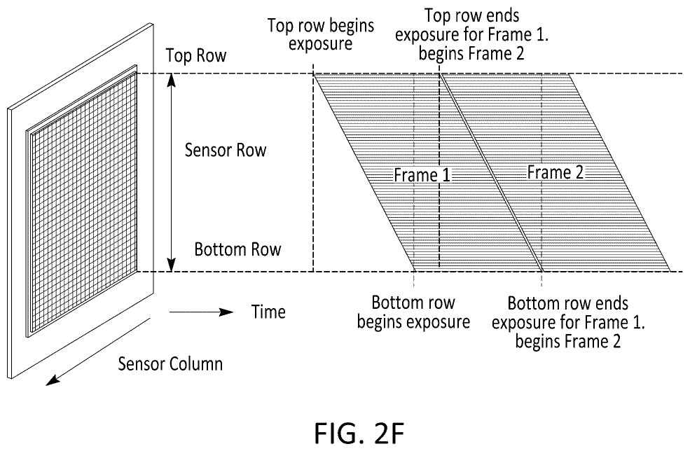

With reference to FIG. 1, aspects of the current subject matter can be implemented using an htSMT workflow where such workflow incorporates systems for image acquisition. For example, such image acquisition can incorporate the imaging of samples to generate a series of images and/or videos. FIG. 2A depicts a schematic of an exemplary image acquisition system of the present disclosure with the X-Z sample plane visible. FIG. 2B depicts the same exemplary image acquisition system, but with the Y-Z sample plane visible. The exemplary image acquisition system (2-001) comprises: a light source (2-005) configured to emit light relayed by one or more optical elements in an optical relay (2-010), the optical relay being configured to shape the light emitted from the light source to form a shaped beam (2-065) such that the shaped beam has a uniform intensity across a longer dimension of the linear shape; an optical element, e.g., a galvo mirror (2-085), configured to translate the shaped beam; and one or more optical elements, e.g., a dichroic mirror (2-100), configured to direct the shaped beam to an objective (2-120), whereby a portion of the sample plane (2-130) is illuminated by an inclined beam (2-125), resulting in the emission of light from the sample, e.g., fluorescence emission, which is focused by the objective (2-120), through a series of optical elements, e.g., a lens (2-155) and an emission filter (2-160), to an image collection system (2-165).

### 2.1.1. Light Source

With reference to the exemplary image acquisition system of FIG. 2A, the system comprises a light source (2-005) configured to emit light. The light source (2-005), in certain implementations of the image acquisition systems disclosed herein, can be configured to emit light of a single wavelength. In certain implementations of the image acquisition systems disclosed herein, the light source (2-005) can be configured to emit light of two, three, four, five, or more individual wavelengths. In certain implementations, the wavelength(s) of light emitted by the light source are predetermined. For example, but not by way of limitation, the wavelength(s) can be predetermined such that the emitted light elicits fluorescence emission when illuminating a sample, e.g., a sample comprising a fluorescent protein. In certain instances, the wavelength(s) employed in connection with the methods described herein will fall within a range of 400 nm to 650 nm. In certain instances, the light source (2-005) will emit light having a wavelength between 400 nm to 408, between 550 nm to 565 nm, or between 638 nm to 650 nm. In certain non-limiting implementations, the light source (2-005) is configured to comprise three lasers with nominal central wavelengths 405 nm, 560 nm, 640 nm that could vary within absorption band of the fluorophores used. In certain instances, the 405 nm wavelength is used to excite Hoechst dye. In certain instances, a 560 nm wavelength is used to excite dyes (e.g., JF₅₄₉) attached to HaloTag. In certain instances, a 642 or a 646 nm wavelength is used to excite dyes (e.g., JF₆₄₆) attached to HaloTag.

In certain non-limiting implementations, the light source (2-005) is used to catalyze photochemical reactions. For example, but not by way of limitation, the wavelength(s) and illumination intensities can be such that cleavage of a chemical bond occurs. As an additional example, but not by way of limitation, the wavelength(s) and illumination intensities may induce the adoption of a non-radiative dark state (i.e., “photobleached molecule”). As an additional example, but not by way of limitation, the wavelength(s) and illumination intensities may induce radiative or non-radiative energy transfer between fluorophores within the sample.

In certain implementations of the image acquisition systems described herein, the light source (2-005) can be configured to deliver a predetermined amount of power to the back focal plane of the objective (2-105). For example, but not by way of limitation, the light source (2-005) delivers greater than 10 mW with respect to certain wavelengths, e.g., 405 nm, and/or greater than 150 mW with respect to other wavelengths, e.g., 640 nm. Additionally, or alternatively, in instances where the light source (2-005) comprises three lasers emitting at 405 nm, 560 nm, and 640 nm wavelengths, respectively the light source (2-005) can be configured to deliver predetermined amounts of power, to the back focal plane of the objective (2-105). For example, but not by way of limitation, the 405 nm can be configured to deliver >10 mW; the 560 nm can be configured to deliver >150 mW; and the 640 nm can be configured to deliver >50 mW).

In certain implementations of the image acquisition systems described herein, the light source (2-005) is configured to emit pulsed light. For example, but not by way of limitation, the light source (2-005) can be configured to emit stroboscopic pulsed light. In certain implementations of the image acquisition systems described herein, the light source (2-005) is configured to emit pulsed light in synchrony with the start of image acquisition. In certain, non-limiting implementations, the light source (2-005) will pulse at specific time intervals depending on the number of frames per second being captured. For example, but not by way of limitation, if 100 Frames Per Second (FPS) are being captured by the detector (2-165), the laser is ON for 9 ms and OFF for 1 ms. In contrast, in 200 FPS mode, the laser is ON for 4 ms OFF for 1 ms. In certain implementations of the OLS htSMT workflow, the light source is configured to go from 90% to 10% power in less than about 0.4 ms. In certain implementations of the OLS htSMT workflow, the light source is configured to go from 90% to 10% power in less than about 0.2 ms.

The emission of light by the light source (2-005) and the direction of that light to the optical relay (2-010), can, in certain implementations of the image acquisition systems disclosed herein, be facilitated using a single mode fiber. Alternatively, a multimode fiber can be employed in certain implementations of the image acquisition systems disclosed herein. For example, but not by way of limitation, the multimode fiber can be configured with a predetermined shape for sample illumination.

In certain implementations of the image acquisition systems described herein, for example with respect to systems configured for high throughput sample analysis, the light source (2-005) can be configured to exhibit low drift in power output. In certain implementations, such low drift configurations increase sample processing consistency to facilitate high throughout analyses. For example, but not by way of limitation, such low drift power output configurations maintain power output within about 0% to about 15% variation, about 0% to about 10% variation, about 10% variation, about 9% variation, about 8% variation, about 7% variation, about 6% variation, about 5% variation, about 4% variation, about 3% variation, about 2% variation or about 1% variation.

In certain instances, such low drift power output configurations maintain power output within about 0% to about 15% variation, about 0% to about 10% variation, about 10% variation, about 9% variation, about 8% variation, about 7% variation, about 6% variation, about 5% variation, about 4% variation, about 3% variation, about 2% variation or about 1% variation in the context of changing ambient (room) temperature, e.g., 17° C.+/−5° C. In certain instances, this is achieved using temperature sensors and/or close-loop heaters to maintain internal light source (e.g., laser engine) temperatures stable, thereby reducing output power drift. For example, but not by way of limitation, the light source can be thermally insulated from the fluctuations of the ambient temperature using an insulated enclosure design. Additionally, or alternatively, closed-loop heaters can be strategically placed at specific locations in the system, e.g., the fiber coupler to reduce output drift. Additionally, or alternatively, water jackets and/or chillers can be used to reduce heat build-up from the laser heads. Moreover, these thermal controls, used individually or in combination, result in shorter warm up times to reach operating steady state and maintained more stable internal operating temperatures when lasers would be powered off and on.

### 2.1.2. Optical Elements & Sample Illumination

With reference to the exemplary image acquisition system of FIG. 2A, the system comprises a light source (2-005) configured to emit light, which is relayed by one or more optical elements in an optical relay (2-010), the optical relay being configured to shape the light emitted from the light source to form a shaped beam (2-065). The particular optical elements of any particular optical relay (2-010) implementation can be selected and configured to produce the appropriately shaped beam (2-065) as well as provide for the appropriate translation of that beam.

In certain, non-limiting, implementations of the optical relay (2-010) of the presently disclosed image acquisition systems, the optical relay (2-010) will comprise one or more lenses and/or other optical elements. For example, but not by way of limitation, the selection and orientation of lenses and other optical elements in the optical relay (2-010) will be configured to appropriately shape the light beam being directed to the sample. In certain non-limiting implementations, the optical relay (2-010) will comprise optical elements to collimate the emitted light, e.g., a collimator (2-020), from the light source (2-005). Additionally, or alternatively, the optical relay (2-010) will comprise additional optical elements, e.g., a Powell lens (2-025) or other elements adapted to produce a beam fan, one or more cylinder lenses ((2-045) and (2-055)), one or more slits to adjust light sheet extent ((2-050) and (2-095)), one or more achromatic lenses ((2-060) and (2-080)) and/or one or more mirrors ((2-070), (2-075) and (2-085)), one or more of which can be a galvo mirror (2-085) capable of translating the light. The particular attributes of the optical element will be predetermined to produce an appropriately shaped light beam. For example, but not by way of limitation, the OLS htSMT systems of the present disclosure can achieve uniform horizontal FOV as well as uniform vertical FOV. Such uniformity in horizontal and vertical FOVs contrasts with other strategies that provide non-uniform horizontal FOV and/or non-uniform vertical FOV (See Table 2.)

TABLE 2
Comparison of Technologies

| | | | Power |
|---|---|---|---|
| | Horizontal | Vertical | Delivery |
| Technology | FOV | FOV | Efficiency |
| HILO | Non-uniform | Non-uniform | Non-efficient |
| HIST | Non-uniform | Uniform | Non-efficient |
| SOLEIL | Non-uniform | Non-uniform | Non-efficient |
| OLS | Uniform | Uniform | Efficient |

To achieve uniform horizontal FOV as well as uniform vertical FOV, the optical relay (2-010) of the OLS htSMT systems described herein comprise an optical element or assembly capable of producing a beam that is elongated along the X plane, and narrow along Y plane and wherein the light beam has a uniform intensity across a longer dimension of the linear shape. In certain non-limiting implementations, the optical relay (2-010) of the OLS htSMT systems described herein will comprise a Powell lens (2-025) to shape the light beam such that it has a uniform intensity across a longer dimension of the linear shape (2-065). The optical relay (2-010) of the OLS htSMT systems described herein can comprise additional or alternative optical elements or assemblies to shape the light beam such that it has a uniform intensity across a longer dimension of the linear shape (2-065). For example, but not by way of limitation, the optical relay (2-010) of the OLS htSMT systems described herein can comprise a diffraction element or assembly configured to shape the light beam such that it has a uniform intensity across a longer dimension of the linear shape.

In certain, non-limiting implementations of the optical relays (2-010) of the presently disclosed image acquisition systems, the optical relay (2-010) will comprise one or more optical elements or assemblies configured to translate the light beam relative to the sample plane of the sample to be analyzed, e.g., in a direction orthogonal to the longer dimension of the light beam. For example, but not by way of limitation, such optical elements or assemblies configured to translate the light beam relative to the sample plane of the sample to be analyzed can comprise a galvo mirror (2-085) or a piezo element configured to translate the light beam. Additionally, or alternatively, such optical elements or assemblies configured to translate the light beam relative to the sample plane of the sample to be analyzed can comprise a computer-controlled motor.

With reference to the exemplary image acquisition system of FIG. 2A, the system comprises an optical relay (2-010) configured to shape the light emitted from the light source to form a shaped beam (2-065), which is then directed by an optical element (2-100), e.g., a dichroic mirror, configured to direct the shaped beam to an objective (2-120), whereby the sample plane (2-130) is illuminated by an inclined beam (2-125).

In certain, non-limiting implementations of the image acquisition systems of the present disclosure, an objective (2-120) directs the inclined beam (2-125) on the sample plane (2-130) to be analyzed. In certain, non-limiting implementations of the image acquisition systems of the present disclosure the objective (2-120) is a water immersion objective. The use of a water immersion objective facilitates high throughput sample analysis by eliminating the oil present in connection with the use of oil immersion objectives, thereby allowing for higher image quality and less distortion. Not only does the presence of oil present issues in the context of automated systems, where the oil can spread to components, including optical elements that can be fouled by exposure to oil, water-immersion objectives are better index-matched for imaging cells, resulting in less distortion and thus higher image quality than with oil objectives. In certain, non-limiting implementations, the objective 60×1.27 NA water immersion objective (Nikon). In certain implementations of the workflows described herein, the water immersion objective (2-120) will be heated by a heating element. For example, such heating element will maintain the water immersion objective (2-120) at a temperature sufficient to avoid inducing a change in temperature of the sample contained in the sample plate (2-021).

### 2.1.3. Image Acquisition

In certain, non-limiting implementations of the image acquisition systems of the present disclosure, the objective (2-0120) is also used to focus the fluorescence emitted by the sample (2-145) in response to the illumination provided by the inclined beam (2-125). In certain, non-limiting implementations, the objective-focused fluorescence emission (2-145) is passed through an emission filter ((2-150) and (2-160)), e.g., a bandpass emission filter matched to the spectrum of the fluorophore under observation and mounted in high-speed filter wheel (Finger Lakes Instruments), and collected by a detector device (2-165). In certain, non-limiting implementations, the objective-focused fluorescence emission is directed to an optical relay prior to collection by the detector device (2-165). For example, but not by way of limitation, such an optical relay can comprise one or more lenses (2-155) and one or more additional optical elements, e.g., an element configured to reject additional scattered light, prior to collection by the detector device (2-165). In certain, non-limiting implementations, the objective-focused fluorescence emission is directed through another diachroic mirror to split the emission over multiple regions of the detector (2-165). In certain, non-limiting implementations, the objective-focused fluorescence emission is directed through another diachroic mirror to split the emission over multiple detectors (2-165).

In certain, non-limiting implementations of the image acquisition systems of the present disclosure, the detector device is configured to synchronize detection with the translation of the inclined beam (2-125) across the sample plane (2-130). Such synchronization is schematically depicted in FIG. 2F. For example, but not by way of limitation, the detector device can be a CMOS camera, e.g., a back illuminated CMOS camera (the Hamamatsu Fusion BT).

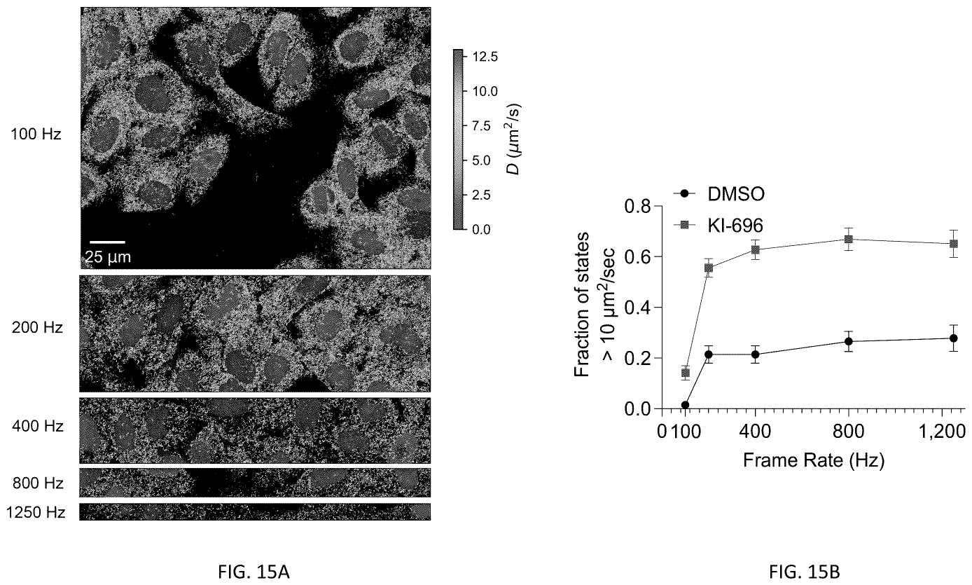

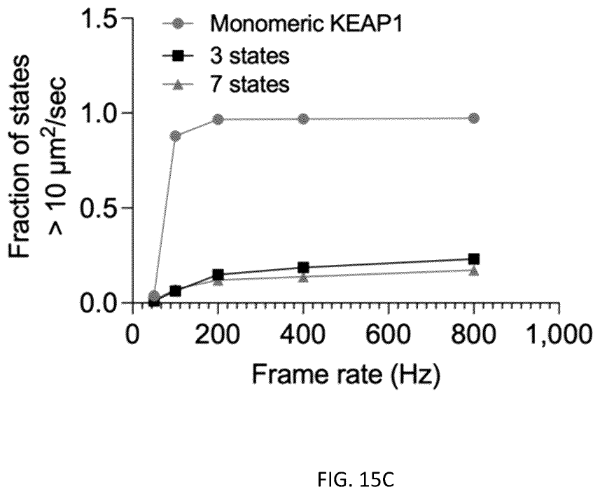

In certain implementations of the image acquisition systems of the present disclosure, the CMOS camera can be run such that, for each field of view, a series of SMT frames are collected. For example, but not by way of limitation, 1-20,000 SMT frames, 1-15,000 SMT frames, 1-10,000 SMT frames, 1-5,000 SMT frames, 1-1,000 SMT frames, 2-500 SMT frames, 5-250 SMT frames, 10-200 SMT frames, 100-200 SMT frames, or 200 SMT frames are collected per field of view. In certain implementations, the CMOS camera can be configured to run at a frame rate from about 0.5 to about 2000 Hz. In certain implementations, the CMOS camera can be configured to run at a frame rate of from 0.5 to 1000 Hz or in certain implementations, at 100 Hz. In certain embodiments, the CMOS camera can be configured to run at a frame rate of from 100 Hz to 1250 Hz as shown in FIGS. 15, 17, 18 and 19. For example, but not by way of limitation, certain cellular SMT implementations can be performed at 100 Hz. In certain embodiments, certain cellular SMT implementations can be performed at 200 Hz. In certain embodiments, certain cellular SMT implementations can be performed at 400 Hz. In certain embodiments, certain cellular SMT implementations can be performed at 800 Hz. In certain embodiments, certain cellular SMT implementations can be performed at 1000 Hz. In certain embodiments, certain cellular SMT implementations can be performed at 1200 Hz. In certain embodiments, certain cellular SMT implementations can be performed at 1250 Hz. In certain embodiments, certain cellular SMT implementations can be performed at 1400 Hz. In certain embodiments, certain cellular SMT implementations can be performed at 1600 Hz. In certain embodiments, certain cellular SMT implementations can be performed at 1800 Hz. In certain embodiments, certain cellular SMT implementations can be performed at 2000 Hz. In certain embodiments, certain cellular SMT implementations can be performed at a frame rate of about 100 Hz or higher, about 200 Hz or higher, about 400 Hz or higher, about 600 Hz or higher, about 800 Hz or higher, about 1000 Hz or higher, about 1200 Hz or higher, about 1400 Hz or higher, about 1600 Hz or higher or about 1800 Hz or higher. In certain embodiments, certain cellular SMT implementations can be performed at a frame rate up to about 1200 Hz. In certain embodiments, certain cellular SMT implementations can be performed at a frame rate up to about 1400 Hz. In certain embodiments, certain cellular SMT implementations can be performed at a frame rate up to about 1600 Hz. In certain embodiments, certain cellular SMT implementations can be performed at a frame rate up to about 1800 Hz. In certain embodiments, certain cellular SMT implementations can be performed at a frame rate up to about 2000 Hz.

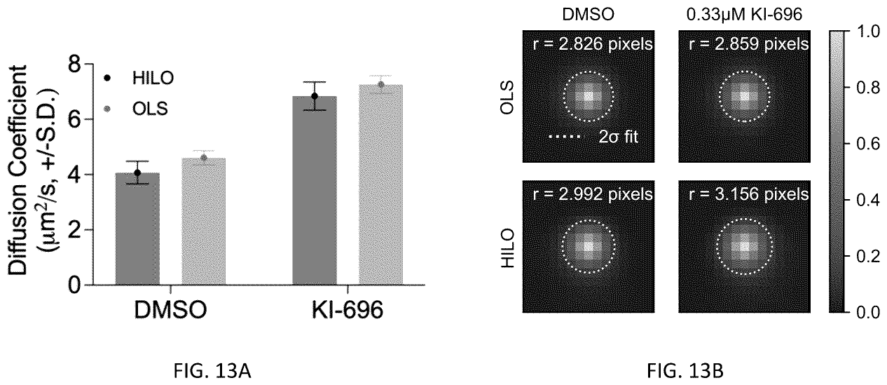

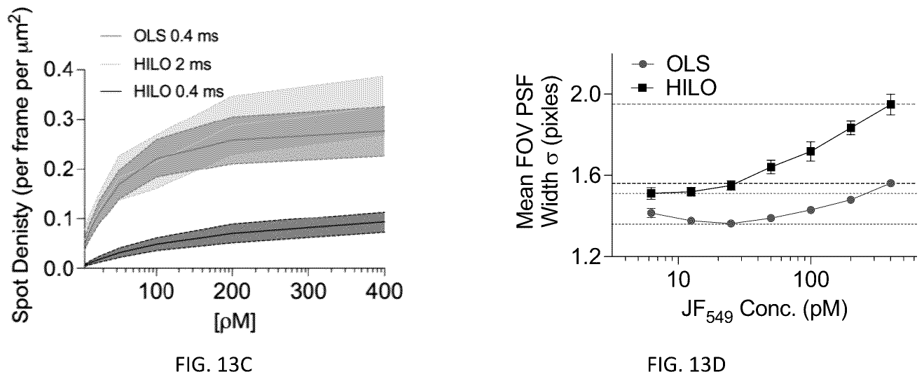

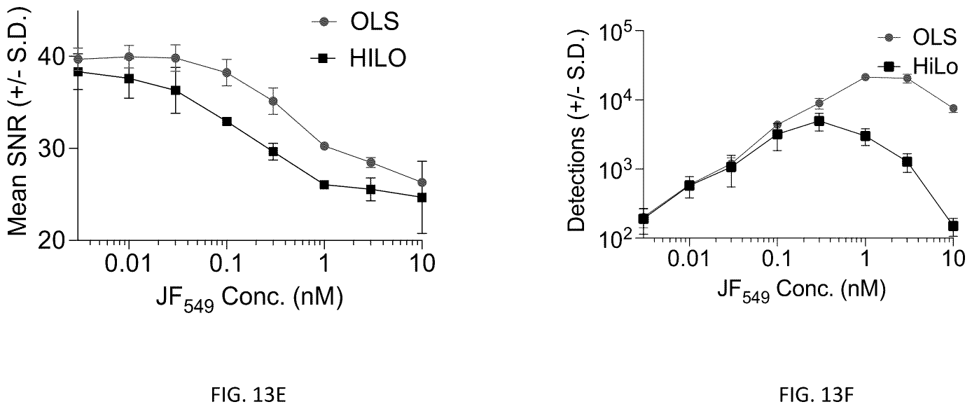

In certain, non-limiting implementations of the image acquisition systems of the present disclosure, the detector device is configured to transmit a signal with each frame to trigger other elements of the imaging system. For example, but not by way of limitation, the detector device may trigger the illumination from the light source (2-005) so as to collect fluorescence emission associated with stroboscopic laser pulses. For example, but not by way of limitation, such fluorescence emission collection is associated with 10 to 100 msec frames and a 2 msec stroboscopic laser pulse. In certain embodiments, fluorescence emission collection is associated with a stroboscopic laser pulse of about 0.1 to about 1 msec. In certain embodiments, fluorescence emission collection is associated with a stroboscopic laser pulse of about 0.2 to about 1 msec, of about 0.3 to about 1 msec, of about 0.4 to about 1 msec, of about 0.1 to about 0.9 msec, of about 0.1 to about 0.8 msec, of about 0.1 to about 0.7 msec, of about 0.1 to about 0.6 msec, of about 0.1 to about 0.5 msec, of about 0.1 to about 0.4 msec, of about 0.2 to about 0.6 msec, of about 0.2 to about 0.5 msec, of about 0.2 to about 0.4 msec or of about 0.3 to about 0.5 msec. In certain embodiments, fluorescence emission collection is associated with a stroboscopic laser pulse of about 0.1 to about 0.6 msec. In certain embodiments, fluorescence emission collection is associated with a stroboscopic laser pulse of about 0.1 to about 0.5 msec. In certain embodiments, fluorescence emission collection is associated with a stroboscopic laser pulse of about 0.2 to about 0.4 msec. In certain embodiments, fluorescence emission collection is associated with a stroboscopic laser pulse of about 0.2 msec. In certain embodiments, fluorescence emission collection is associated with a stroboscopic laser pulse of about 0.4 msec as shown in FIG. 13C.

In certain implementations, the imaging acquisition system can be configured to acquire a predetermined field of view (FOV), e.g., a detected FOV. In certain embodiments, the FOV, e.g., detected FOV, can have a size of about 150 μm to about 250 μm in a first dimension by about 100 μm to about 210 μm in a second dimension. In certain embodiments, the FOV, e.g., detected FOV, can have a size of about 200 μm to about 250 μm in a first dimension by about 150 μm to about 210 μm in a second dimension or the FOV, e.g., detected FOV, can have a size of about 225 μm to about 250 μm in a first dimension by about 175 μm to about 210 μm in a second dimension. For example, but not by way of limitation, the FOV, e.g., detected FOV, can have a size of about 250 μm in a first dimension by about 190 μm in a second dimension, e.g., as disclosed in Example 1.

In certain embodiments, a certain percentage of an FOV, e.g., a detected FOV, provides useable data. In certain embodiments, at least 75% of the FOV, at least 80% of the FOV, at least 85% of the FOV, at least 90% of the FOV, at least 95% of the FOV, at least 96% of the FOV, at least 97% of the FOV, at least 98% of the FOV, at least 99% of the FOV or 100% of the FOV provides useable data. In certain embodiments, at least 75% of the FOV, e.g., detected FOV, provides useable data. In certain embodiments, at least 80% of the FOV, e.g., detected FOV, provides useable data. In certain embodiments, at least 85% of the FOV, e.g., detected FOV, provides useable data. In certain embodiments, at least 90% of the FOV, e.g., detected FOV, provides useable data. In certain embodiments, at least 95% of the FOV, e.g., detected FOV, provides useable data. In certain embodiments, at least 96% of the FOV, e.g., detected FOV, provides useable data. In certain embodiments, at least 97% of the FOV, e.g., detected FOV, provides useable data. In certain embodiments, at least 98% of the FOV, e.g., detected FOV, provides useable data. In certain embodiments, at least 99% of the FOV, e.g., detected FOV, provides useable data. In certain embodiments, 100% of the FOV, e.g., detected FOV, provides useable data. In certain embodiments, a percentage equal to or greater than about 75% of the FOV provides useable data, e.g., a percentage equal to or greater than about 80% of the FOV, a percentage equal to or greater than about 85% of the FOV, a percentage equal to or greater than about 90% of the FOV, a percentage equal to or greater than about 95% of the FOV, a percentage equal to or greater than about 96% of the FOV, a percentage equal to or greater than about 97% of the FOV, a percentage equal to or greater than about 98% of the FOV or a percentage equal to or greater than about 99% of the FOV provides useable data. In certain embodiments, a certain percentage of an FOV, e.g., a detected FOV, achieves sufficient laser illumination for tracking protein movement. For example, but not by way of limitation, at least 75% of the FOV, at least 80% of the FOV, at least 85% of the FOV, at least 90% of the FOV, at least 95% of the FOV, at least 96% of the FOV, at least 97% of the FOV, at least 98% of the FOV, at least 99% of the FOV or 100% of the FOV achieves sufficient laser illumination for tracking protein movement. In certain embodiments, at least 75% of the FOV, e.g., detected FOV, achieves sufficient laser illumination for tracking protein movement. In certain embodiments, at least 80% of the FOV, e.g., detected FOV, achieves sufficient laser illumination for tracking protein movement. In certain embodiments, at least 85% of the FOV, e.g., detected FOV, achieves sufficient laser illumination for tracking protein movement. In certain embodiments, at least 90% of the FOV, e.g., detected FOV, achieves sufficient laser illumination for tracking protein movement. In certain embodiments, at least 95% of the FOV, e.g., detected FOV, achieves sufficient laser illumination for tracking protein movement. In certain embodiments, at least 96% of the FOV, e.g., detected FOV, achieves sufficient laser illumination for tracking protein movement. In certain embodiments, at least 97% of the FOV, e.g., detected FOV, achieves sufficient laser illumination for tracking protein movement. In certain embodiments, at least 98% of the FOV, e.g., detected FOV, achieves sufficient laser illumination for tracking protein movement. In certain embodiments, at least 99% of the FOV, e.g., detected FOV, achieves sufficient laser illumination for tracking protein movement. In certain embodiments, 100% of the FOV, e.g., detected FOV, achieves sufficient laser illumination for tracking protein movement. In certain embodiments, a percentage equal to or greater than about 75% of the FOV achieves sufficient laser illumination for tracking protein movement, e.g., a percentage equal to or greater than about 80% of the FOV, a percentage equal to or greater than about 85% of the FOV, a percentage equal to or greater than about 90% of the FOV, a percentage equal to or greater than about 95% of the FOV, a percentage equal to or greater than about 96% of the FOV, a percentage equal to or greater than about 97% of the FOV, a percentage equal to or greater than about 98% of the FOV or a percentage equal to or greater than about 99% of the FOV provides sufficient laser illumination for tracking protein movement.

In certain implementations, the imaging acquisition system can be configured to acquire a predetermined image dimension per frame, referred to herein as the Region of Interest (ROI). In certain implementations, the ROI will differ depending on the frame rate employed. For example, at 100 FPS, 2304×1728 pixels will define the ROI which amounts to 248.832×186.624 microns in the sample plane. In contrast, at 200 FPS, 2304×768 pixels will define the ROI, which amounts to 248.832×82.944 microns in the sample plane.

In certain implementations, the imaging acquisition system can be configured to perform predetermined sweep rates at predetermined frame rates. For example, but not by way of limitation, at 100FPS: the sweep rate can be 186.624 microns/9 ms, which is equivalent to 20.8 microns/ms, which is equivalent to 2.08 cm/s. In contrast, at 200FPS the sweep rate can be 82.94 microns/4 ms which is equivalent to 20.7 microns/ms which is equivalent to 2.07 cm/s.

In certain implementations, the detector device can be used to collect fluorescence emission at a multiple wavelengths. For example, but not by way of limitation, fluorescence emission of additional fluorophores can be collected at the same frame rate or different frame rates for the same fields of view to provide downstream registration of SMT tracks to other cellular components, e.g., nuclei. Additional channels of the detector device can be used as desired to expand the number of simultaneously captured fluorescence emissions for the same fields of view to provide downstream registration of SMT tracks to other cellular components, e.g., nuclei.

### 2.2. Sample Handling

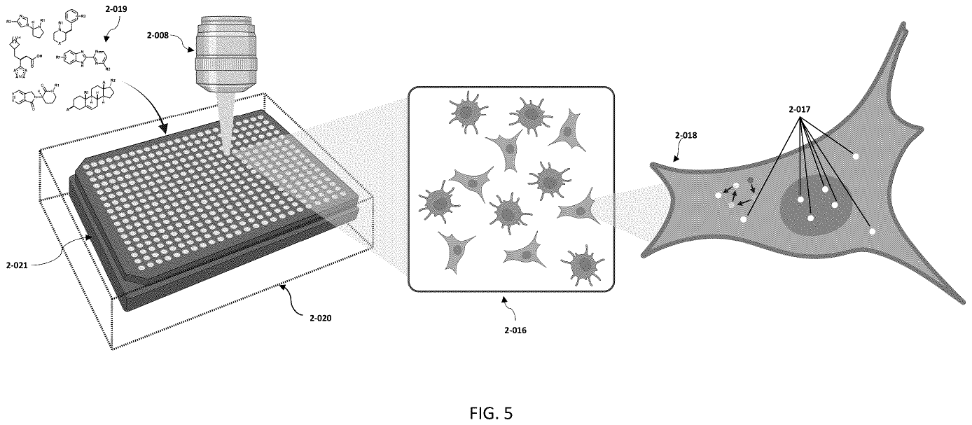

With reference to FIG. 1, aspects of the current subject matter can be implemented using an htSMT workflow, where such workflow incorporates systems for sample preparation, including reagent handling. For example, but not by way of limitation, FIG. 5 provides a schematic representation of a sample plate (2-021) comprising a plurality of wells (2-016) in which samples can be prepared and analyzed. FIG. 5 also provides a schematic representation of components of a sample, e.g., a cell (2-018) and fluorescent target proteins (2-017) within the cell. As noted herein, however, FIG. 5 is not intended to convey scale, e.g., each sample present in a well (2-016) can comprise thousands of cells and each cell can comprise numerous fluorescent target proteins. FIG. 5 also schematically illustrates the ability of sample handling systems of the present disclosure to add additional reagents to samples (2-019). Such reagent addition can be handled by robotic manipulations, such as, but not limited to, the translation of robotic fluid handling systems relative to the individual wells (2-016) of the sample plate (2-021), the translation of the sample plate (2-021) itself, or combinations of both. In certain implementations of the image acquisition system, the sample plate (2-021) may be maintained in a temperature-controlled environment through an environmental control area (2-020). For example, but not by way of limitation, the sample may be maintained at 22-50° C. In certain implementations of the image acquisition system, the sample plate (2-021) can be maintained in a humidity-controlled environment through an environmental control area (2-020). For example, but not by way of limitation, the sample may be maintained at 20%-95% humidity. In certain implementations of the image acquisition system, the sample plate (2-021) may be maintained in a defined gas environment through an environmental control area (2-020). For example, but not by way of limitation, the sample may be maintained at 5% CO₂.

### 2.2.1. Cell Lines & Cell Culture

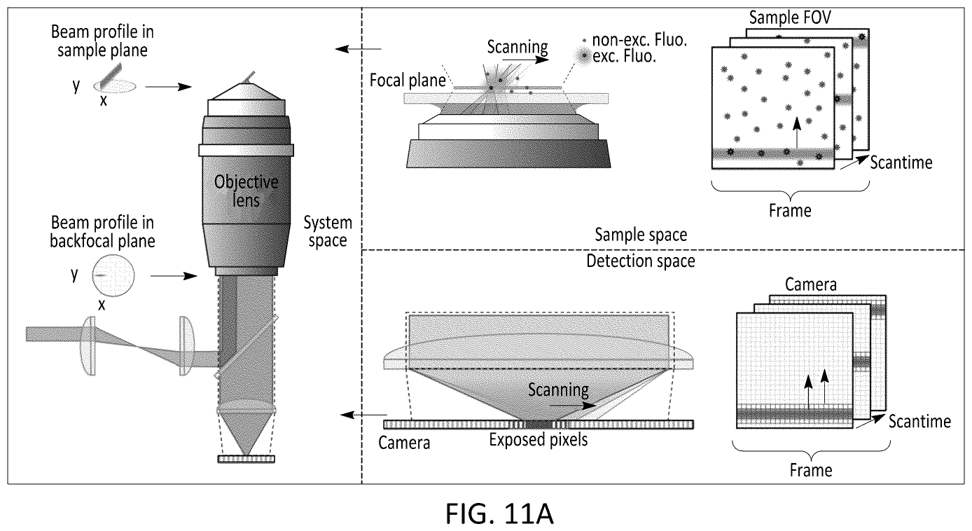

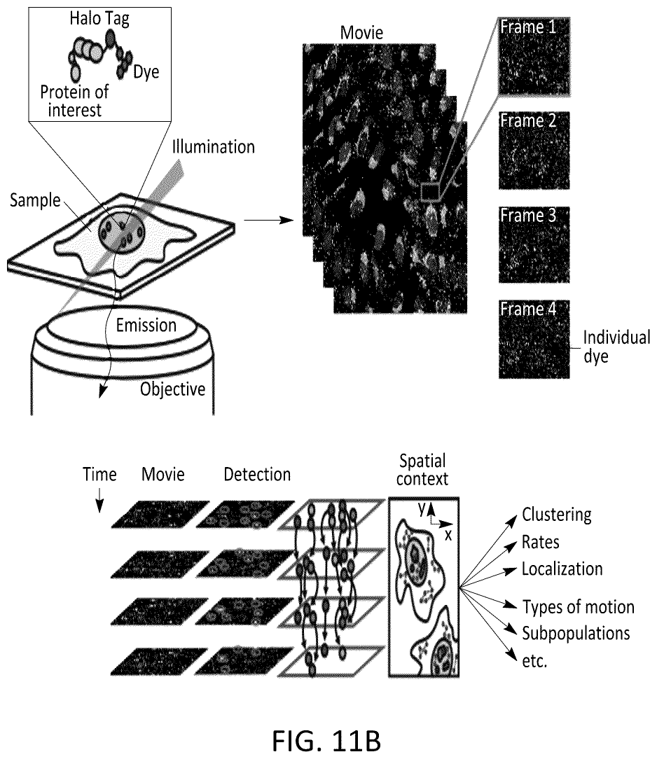

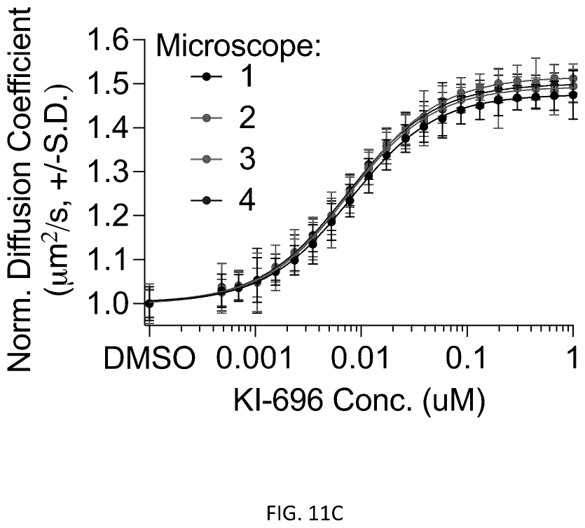

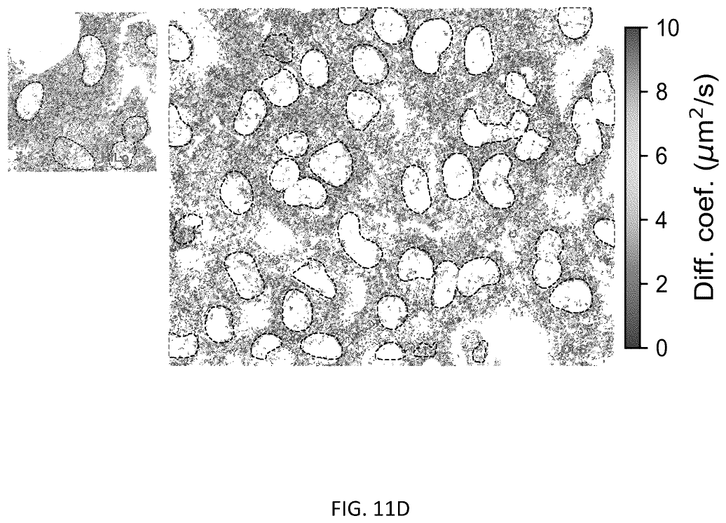

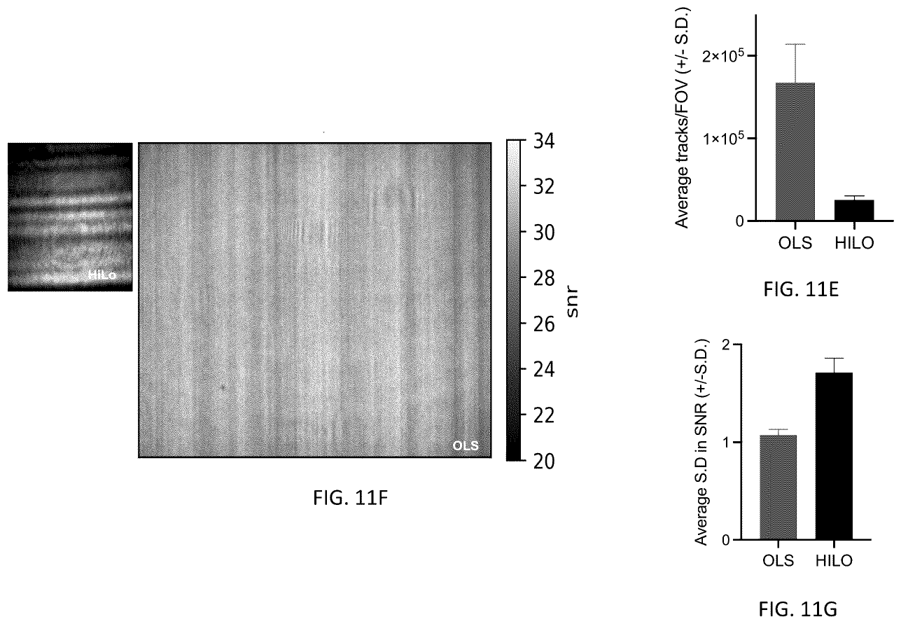

With reference to FIG. 5, a particular advantage of the htSMT systems described herein is that living cells (2-016) can be assayed to facilitate the tracking of activity, mobility, and diffusive behaviors of proteins within the crowded living cellular environment. As shown in FIG. 11B, the htSMT systems of the present disclosure can be used to track fluorescently labeled proteins in a sample comprising a plurality of cells. Exemplary cells (e.g., cell lines) that find use in connection with the htSMT systems described herein are considered if the sample (e.g., containing such cells) can be brought into focus by the objective (2-120) for sufficient time as to direct the fluorescence emission of fluorophores onto the detector (2-165). For example, but not by way of limitation, cells may adhere to coverglass directly. As an additional example, but not by way of limitation, cells may be induced to adhere to the coverglass after treating the coverglass with an extracellular matrix material (e.g., fibronectin, collagen, poly-D-lysine, laminin, matrigel, vitronectin, etc.). As an additional example, but not by way of limitation, cells may be induced to adhere to the coverglass after treating the coverglass with plasma.

Exemplary cells, e.g., cell lines, may be selected so as to minimize non-fluorophore emissions reaching the detector. In certain embodiments, cells for use in the present disclosure can be mammalian, bacterial or fungal cells. In certain embodiments, the cells are mammalian cells. In certain embodiments, the cells can be obtained from preserved tissue, e.g., fixed tissue, from frozen tissue e.g., frozen tissue samples, or from fresh tissue, e.g., fresh tissue samples. In certain embodiments, the cells and/or a sample containing cells can be obtained from a subject. In certain embodiments, the cells can be obtained from a malignancy of a tissue or a tumor, e.g., the cells can be present within a tumor sample (e.g., a section of a tumor). In certain embodiments, the cells can be obtained from cell lines. For example, but not by way of limitation, particular cell lines that find use in connection with the htSMT systems described herein included: U2OS cells (ATCC Cat. No. HTB-96), MCF7 cells (ATCC Cat. No. HTB-22), T47d cells (ATCC Cat. No. HTB-133) and SK-BR-3 cells (ATCC Cat. No. HTB-30). In certain embodiments, the cells can be present in a three-dimensional structure such as an organoid or a spheroid. In certain embodiments, the cells can be present in an organoid.

In certain implementations of the htSMT systems of the present disclosure, the cells to be used are cultured as necessary to provide sufficient cell numbers to achieve the desired high throughput analyses. For example, but not by way of limitation, cells, e.g., U2OS cells (ATCC Cat. No. HTB-96), MCF7 cells (ATCC Cat. No. HTB-22), T47d cells (ATCC Cat. No. HTB-133) and SK-BR-3 cells (ATCC Cat. No. HTB-30), can be grown in DMEM (Cat. No. 1056601, Gibco DMEM, high glucose, GlutaMAX Supplement, Thermofisher) supplemented with 10% Fetal Bovine Serum (Cat. No. 16000044, Thermofisher) and 1% pen-strep (Cat. No 15140122, Thermo Fisher) and maintained in a humidified 37° C. incubator at 5% CO₂ and subcultivated approximately every two to three days. Additional culture strategies that would be appropriate for the cell lines and uses outlined herein would be known those of skill in the relevant art.

In certain implementations of the htSMT systems of the present disclosure, the cells comprise one or more fluorescent target protein. The selection of the specific protein(s) to be labeled and the specific labeling approach will likely differ depending on the particularities of a specific investigation. For example, but not by way of limitation, one approach for labeling proteins that finds use in connection with the htSMT systems described herein is a HaloTag fusion strategy. For example, but not by way of limitation, one approach for labeling proteins is a SNAPtag fusion. For example, but not by way of limitation, one approach for labeling proteins is a CLIPtag fusion. For example, but not by way of limitation, one approach for labeling proteins is through a fluorophore ligase system. For example, but not by way of limitation, one approach for labeling proteins is via FLASH or ReAsH tetracysteine motif. For example, but not by way of limitation, one approach for labeling proteins is through strain-promoted alkyne-azide cycloaddition of a fluorophore. For example, but not by way of limitation, one approach for labeling proteins is through inducing cellular uptake of fluorescent target proteins generated separately. In certain implementations of the htSMT systems of the present disclosure, the cells comprise one or more fluorescently labeled glycoprotein. In certain embodiments, one approach for labeling proteins uses a gene-editing system, e.g., a CRISPR-based editing system. For example, and not way of limitation, a nucleic acid encoding a fluorescent protein (e.g., a fluorescent tag such as a HaloTag) can be inserted into the gene or upstream or downstream from the gene encoding the protein to be labeled to generate a protein that is fluorescently labeled with a HaloTag (e.g., at its C- or N-terminus), e.g., as described in Example 2.

While one of skill in the art can implement a HaloTag fusion-approach in a number of ways, one exemplary approach is to transfect mammalian expression vectors containing the fusion gene (i.e., a protein of interest fused in frame with a HaloTag sequence) under the control of a weak L30 promoter and containing a Neomycin resistance marker in the cell line of interest, e.g., U2OS cells. In certain implementations, such transfection can be accomplished when the cells are at 70% confluence using FuGENE 6 (Cat. No. E2691, Promega). In certain implementations, transfected cells can then be selected with the appropriate selection agent, e.g., G418 (Cat. No. 10131027, Thermo Fisher), at the appropriate concentration, e.g., at 500 μg/mL. In certain implementations, cells can then be clonally isolated. Clones expressing the desired fusion gene can be determined first by staining with 100 nM JF₅₄₉-HTL (Cat. No. GA1110, Promega) and 50 nM Hoechst 33342 and identifying clones with the expected distribution of JF₅₄₉ signal. An alternative exemplary approach it to transfect cells with ribonucleoprotein (RNP) complexes including sgRNAs targeting a genomic sequence encoding the N- or C-terminal region of a target protein and Cas9 protein in combination with one or more linear dsDNA donors. In certain embodiments, each donor consists of 200-300 bp homology arms specific for each target, a codon optimized HaloTag sequence and TEV linker (ENLYFQG) between the target and HaloTag. In certain implementations, between three and six clones can be subsequently tested using SMT conditions for response to a control compound, and the most homogenous clones can then be subsequently expanded for further testing.

While the htSMT workflows of the instant application are described generally with respect to implementations that track the impact of a compound on a target fluorescent protein, the htSMT workflows described herein are equally applicable to the tracking and analysis of fluorescent target compounds. For example, but not by way of limitation, the compounds described herein can either themselves be fluorescent or can be modified to facilitate fluorescent detection. Moreover, changes in the movement of the fluorescent compound can be utilized to determine the SMT profile of the compound itself. All analysis strategies described herein with respect to the tracking of target fluorescent proteins are therefore also applicable to results obtained by tracking the compounds themselves.

### 2.2.2. Single Molecule Tracking Sample Preparation

With reference to FIG. 5, aspects of the current subject matter can be implemented using an htSMT workflow whereby cells (2-018) are seeded on plates (2-021), e.g., tissue culture treated 384-well glass-bottom plates, although other plate types can find use in connection with the approaches outlined herein, including, but not limited to single chambers, 9-well glass-bottom plates, 24-well glass-bottom plates, 96-well glass-bottom plates, 1536-well glass-bottom plates, and 3456-well glass bottom plates, as well as plates made of alternative materials, e.g., plates made partially or entirely of plastic. In certain implementations, the cells (2-018) are seeded at 1 to 20,000 cells per well (2-016), e.g., at 50 to 10,000, at 100 to 9,000, at 250 to 8500, at 500 to 7500, at 750 to 7000, at 2500 to 6500, or at 6000 cells per well. Seeded cells can then be incubated under conditions desirable for adhesion, e.g., overnight at 37° C. and 5% CO₂. To enable fluorescence emission, cells can be incubated with a sufficient amount of label, e.g., one or more cell permeable fluorophores. For example, but not by way of limitation, in the case of HaloTag fusions, cells can be incubated with about 0.1 to about 100 pM of JF₅₄₉, JF₆₄₆ or other comparable cell permeable fluorophore. In certain embodiments, cells can be incubated with about 0.1-100 pM of JF₅₄₉-HTL (Cat. No. GA1110, Promega) or about 0.1-100 pM of JF₆₄₆ and/or 50 nM Hoechst 33342 (for labeling nuclei), e.g., for an hour in complete medium can provide desirable results.

In certain implementations htSMT strategies described herein, the cells are then washed, e.g., three times in DPBS and twice in imaging media. In certain implementations, the imaging media is prepared to facilitate fluorescence emission, e.g., fluoroBrite DMEM media (Cat. No. A1896701, Thermo Fisher), and can be supplemented with GlutaMAX (Cat. No. 35050079, Thermo Fisher) and the same serum and antibiotics as growth media.

Where appropriate, compounds can be added to the samples to test their impact on a particular labeled protein via SMT. In certain implementations, compounds can be serially diluted in an Echo Qualified 384-Well Low Dead Volume Source Microplate (0018544, Beckman Coulter) to generate dose-titration source material. Compounds can then be administered, e.g., at a final 1:1000 dilution in cell culture medium. In certain implementations of the htSMT strategies described herein, each dose of a compound will have at least two replicates per plate as well as three plate replicates. In addition, in certain implementations of the htSMT strategies described herein, 20 DMSO control wells and two no dye control wells can be randomized across each sample plate (2-020). In certain implementations, compounds can be allowed to incubate for 0 to 48 hours prior to image acquisition, e.g., one hour at 37° C.

### 3. OLS HTSMT SOFTWARE

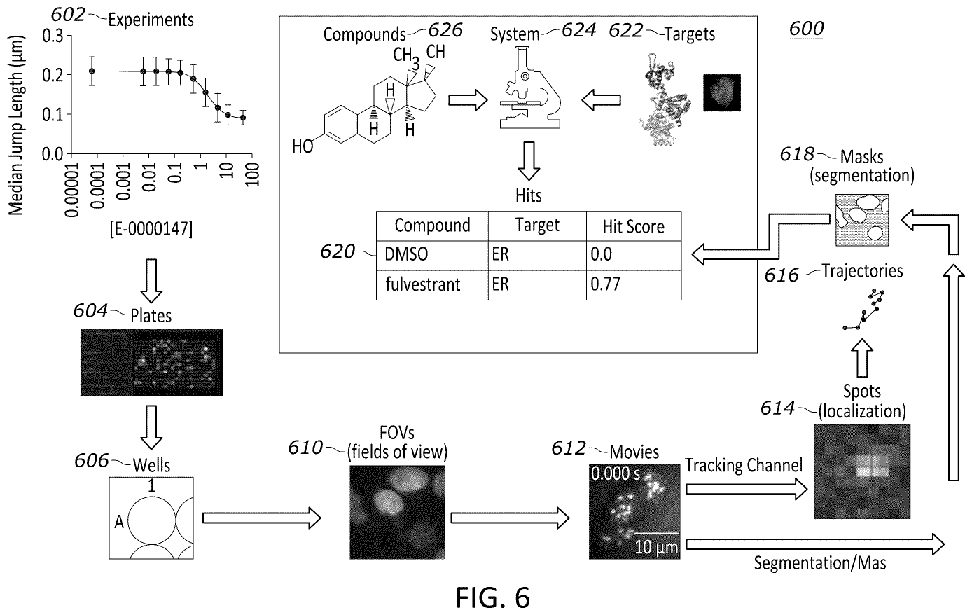

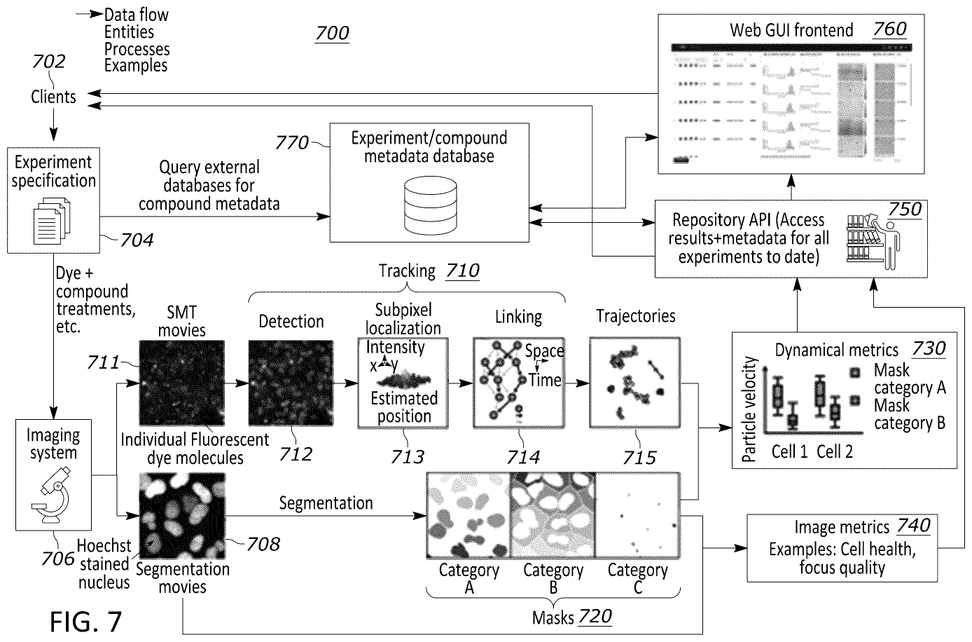

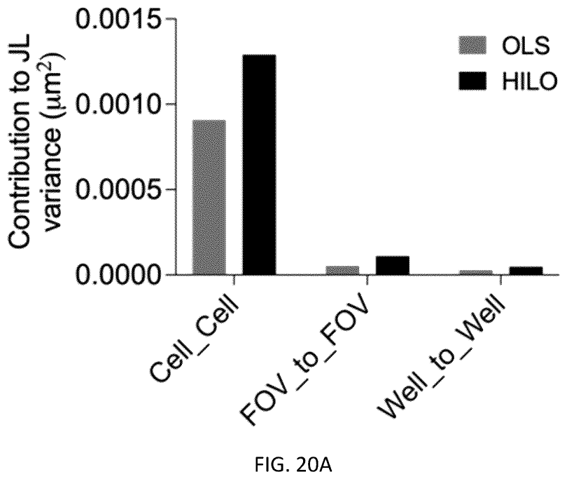

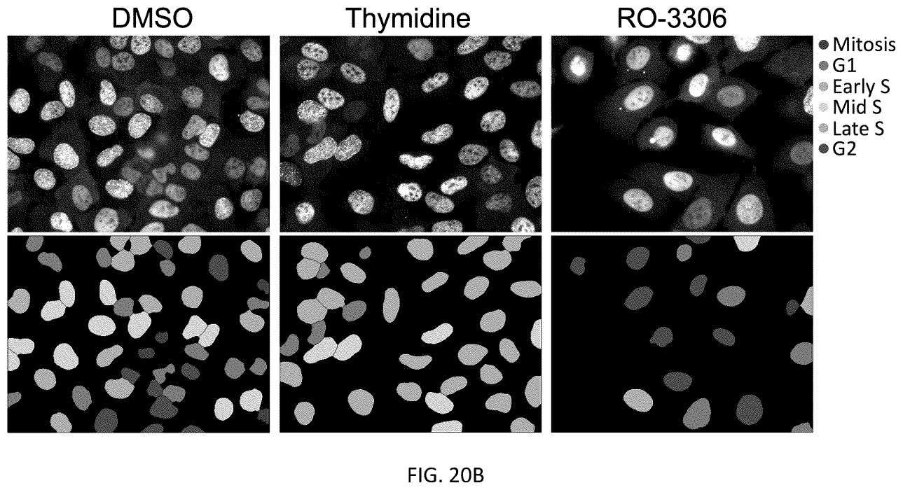

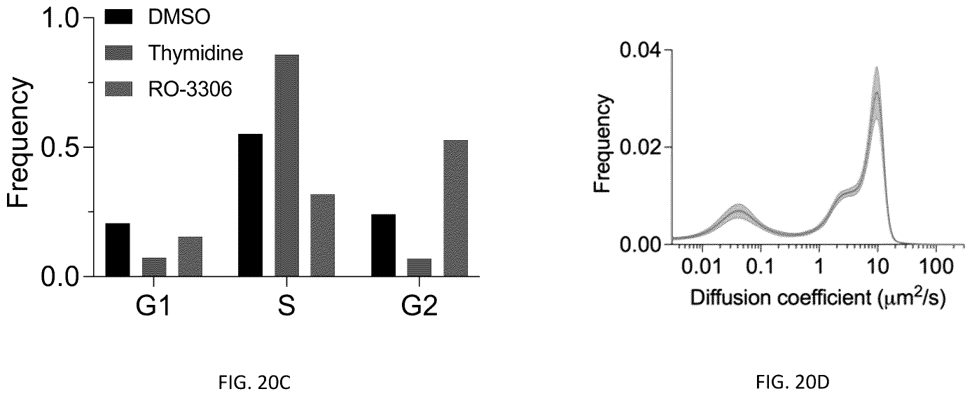

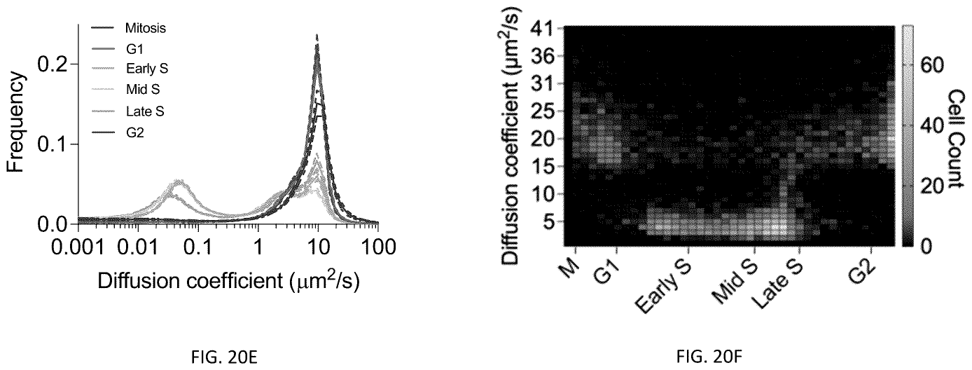

FIG. 6 illustrates an example system 600 for a high-throughput single-molecule imaging platform that measures molecule movement in living cells. Experiments 602 can be performed to collect large amounts of data from a plurality of living cells (e.g., using imaging system 624 to identify compounds 626 and/or targets 622). The experiments 602 can include the application of various identifiers to molecules of interest such as labels which can be subsequently fluoresced or otherwise detected (e.g., using a laser or other light source). The biological samples forming part of such experiments 602 can be organized into plates 604 having a plurality of wells 606. Each well 606 can have one or more associated fields of view (FOVs) 610. FOVs 610 can be locations within or corresponding to a single well 606. A sequence of images can be generated for the FOVs 610 to result in one or more movies 612, which can include SMT movies as well as non-SMT movies. SMT movies can be used to track the paths of individual labeled molecules such as proteins, generating a plurality of trajectories. Each trajectory may be comprised of a plurality of spots 614, which include the spatiotemporal coordinates of a labeled molecule at a particular time (as described in further detail in FIG. 7). Separately from the tracking, and in some instances in parallel with the tracking, the movies 612 can be utilized to identify molecules through the use of machine-learning and/or computer vision-based image segmentation to generate masks 618. Masks 618 are spatial regions within a FOV 610 produced by the segmentation. Each mask 618 can belong to a mask category, which is described in more detail in FIG. 8 and FIG. 20B.

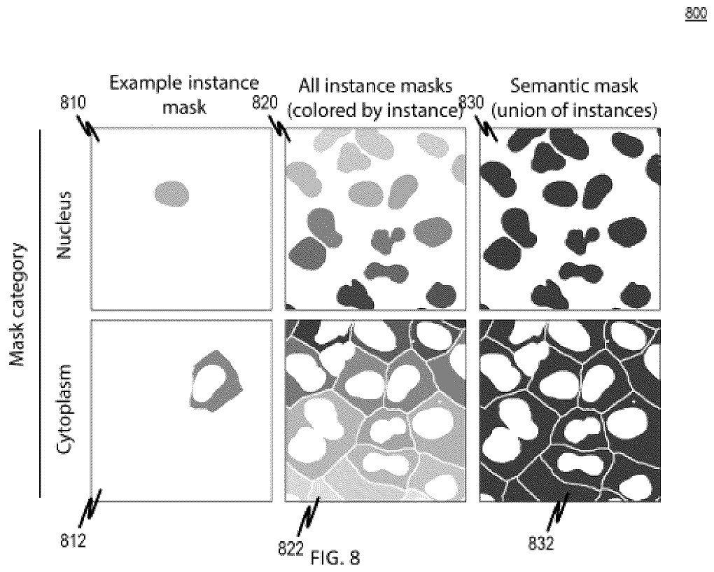

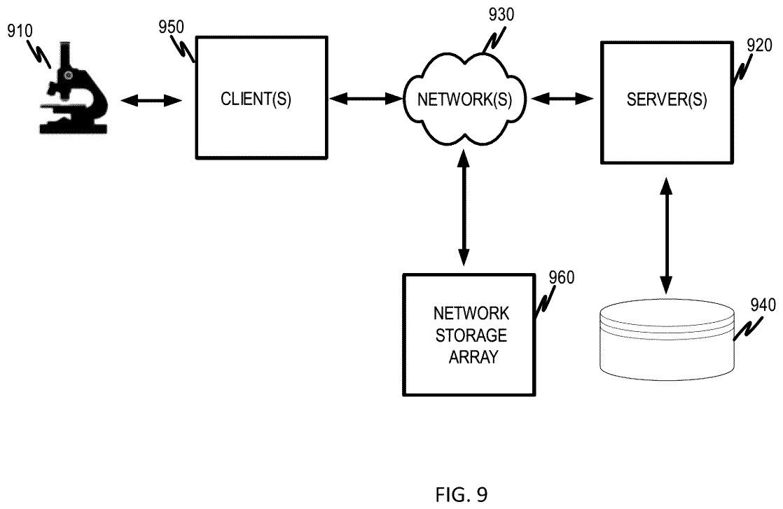

Data associated with two channels (e.g., tracking channel and segmentation/masking channel) can be combined to generate a plurality of metrics 620 associated with various aspects of the samples. In other words, the trajectories 616 (e.g., trajectory data) can be combined with the machine learning processed image segmentation data and further analyzed using statistical/machine learning methods. Processing of the combined data can be used to generate metrics 620 such as hit scores associated with compounds and/or targets within a biological sample that may be stored in a database structure, as further described in FIG. 9.

FIG. 7 illustrates data flow through an example system 700 for a high-throughput single-molecule imaging platform that measures protein movement in living cells. Experiment specifications 704 that define experiments 602 can be provided as data input via one or more clients 702. For example, each experiment 602 can be collected with accompanying stains (e.g., Hoechst or Potomac Red) that are used for downstream analysis including segmentation 618. The experiment specifications 704 can define various parameters for the experiments 602 such as stains, dyes, compounds, treatments, and the like. As previously described in FIG. 6, imaging system 706 (e.g., imaging system 624) can capture a sequence of images that generate one or SMT movies 711 and/or non-SMT movies or segmentation movies 708 (e.g., movies 612) which characterize molecular movement. The SMT movies 711 can characterize movement of individual fluorescent molecules and/or contain images of individual fluorescent molecules. The segmentation movies 708 can comprise a sequence of images that characterize movement of labeled molecules and/or component thereof. It will be appreciated that Hoechst staining is only one technique that can be used to label molecules and that different and/or multiple labeling techniques such as Potomoc Red can be utilized depending on the desired configuration. For example, MitoTracker Deep Red can be used to label mitochondria), concanavalin A-dye conjugates can be used to label endoplasmic reticulum, SYTO 14 can be used to label nucleoli, phalloidin can be used to label actin, and the like.

The SMT movies 711 can be analyzed to perform operations relating to molecule tracking 710 which can include detecting 712, subpixel localization 713, and linking 714 to identify trajectories 715 of molecules across various images within the SMT movies 711. More specifically, during detection 712 one or more spots within the SMT movies 711 can be detected or recovered. Each spot can be equipped with spatiotemporal coordinates. These spatiotemporal coordinates can be estimated by using subpixel localization techniques 713. Linking 714 can be performed on the spots to ultimately identify trajectories 715.

Links, as used herein, are potential associations between two spots. Each link is directed, beginning at one spot and ending at another. A “correct link” joins two spots produced by the same emitter in different frames; otherwise, a link is “incorrect.” One objective of the linking algorithm is to estimate which links are correct. Links are referred to herein in the format α: i→j This is taken to mean: link a, which begins at spot i and ends at spot j. Links satisfy at least three of the following constraints: (a) links go forward in time, (b) links may not join two spots that are farther apart than some limit (referred to herein as the “search radius”), and (c) links may not join two spots that are temporally separated by more than some limit (referred to herein as the “gap limit”). A spot-link graph is a graph of spots and links for one SMT movie 711. The spots are the vertices and the links are the edges of this graph. Because links go forward in time, the spot-link graph is a directed acyclic graph. A matching is a subset of the links in a spot-link graph such that no two links in this subset begin or end at the same spot. Trajectories 715 are used herein to refer to sequences of contiguous (end-to-end) links in the same matching. Dynamical metrics 730 can be determined using a plurality of trajectories. Such parameters can comprise attributes of a spot that characterize the spot's movement. Such parameters can comprise one or more of velocity, diffusion coefficient, or anomaly parameter(s) for each spot. The dynamical parameter(s) for spot i are herein referred to as θᵢ. The set of dynamical parameters for all spots in a spot-link graph are herein referred to as Θ.

Separate from, and in some variations in parallel with, the processing of SMT movies 711, segmentation movies 708 can undergo segmentation, which generates one or more masks 720. The masks can be of various categories, including but not limited to, cell nuclei, cell cytoplasm, cell cycle phase and/or extraneous masks, which are further described in FIG. 8 and FIG. 20B. Instance masks are individual segmented objects (e.g., one cell, one nucleus, one mitochondrion, M phase, G1 phase, Early S phase, Middle S phase, Late phase, G2 phase). A FOV 610 may contain any number of instance masks for one mask category. Semantic masks are the union of all instance masks corresponding to one type of mask category for one FOV (e.g., all cells, all nuclei, or all mitochondria for one FOV, etc.). The extraneous masks can contain parts of the non-SMT movie 708 that are excluded from any downstream data analysis. For example, these extraneous masks could correspond to parts of the non-SMT movie 708 that are out of focus or that contain auto fluorescent cell debris that prevents accurate tracking. During segmentation, molecules within the segmentation movies 708 can be assigned to one or more masks. Image metrics 740 can be evaluated from the masked molecules such as cell health, focus quality, or the like.

Experiment information such as the dynamical metrics 730, the image metrics 740, and any data from which either metric is derived (e.g., segmentation information) can be provided to a data repository 770 for storage. Such data repository 770 can store, for example, any results of experiment 602 such as the dynamical metrics 730, image metrics 740, and/or any data from which either metric is derived. Data repository can comprise local persistence and/or dedicated servers accessed locally or by way of the cloud. Data repository 770 can also store metadata associated therewith and/or metadata associated with the experiment specification 704. The experiment information (e.g., results and metadata from historical experiments, etc.) can be provided to data repository 770 via a repository application program interface (API) 750. The repository API 750 can also interface with a web-based graphical user interface front end 760 that provides such information for display on clients 702.

In some variations, segmentation information can be used to identify subcellular compartments such as nuclei, nucleoli, cytoplasm, and the like. Segmentation information can also be used to distinguish one cell from another. Segmentation information can be stored in a specific format (e.g., a multi-image file format such as TIFF, etc.).

Example dynamical metrics 730 can also include state arrays. State arrays are a framework for learning interpretable dynamical models from SMT trajectories, and can be used for gaining additional insight into the movement of a target protein and where in the cell that movement occurs. In some variations, state arrays can be generated/populated using the segmentation information. The outputs for state arrays can be returned at the subcellular compartment level, allowing scientists to distinguish dynamics in different subcellular compartments. Additionally, state arrays can be computed on each individual subcellular compartment (e.g., per nucleus).

To facilitate data access by applications, including but not limited to state arrays, processed SMT data may be stored in a format that permits (a) representation of processed trajectories and associated attributes such as SNR and spot shape characteristics for each SMT movie, (b) representation of mask objects, including mask category (e.g., each mask object's associated subcellular organelle, each mask object's cell cycle phase, etc.), (c) association of trajectories with mask objects (such as the cell nucleus in which each trajectory was observed or the cell cycle phase in which each trajectory was observed), and (d) association of all SMT movies with metadata relevant to the original experiment, such as compound treatments, acquisition times, and imaging system name. Formats (a) and (c) can be a Protocol Buffer schema defining a storage format for trajectories along with associated mask objects. Format (b) can be a specialized image file format that includes the mask objects to which each pixel in an FOV belongs. Format (d) may be a PostgreSQL database that records all captured experiments/movies. As a client of processed SMT data, state arrays can draw on these data schemas to report dynamic characteristics of trajectories on a per-mask category or per-mask object basis.

FIG. 8 is a plurality of images 800 illustrating differences between mask categories and instance or semantic masks. As previously discussed, non-SMT movies or segmentation movies can be assigned to a plurality of categories. Such categories can include cell nuclei (e.g., Category A), cell cytoplasm (e.g., Category B), and/or extraneous masks (e.g., Category C). Unique, individual masks can be applied to biological samples. For example, image 810 is of a unique, individual instance mask applied to a cell nucleus (e.g., Category A). Image 812 is of a unique, individual instance mask applied to a cell cytoplasm (e.g., Category B). Image 820 illustrates multiple instance masks applied to one or more nuclei, with individual colors representing a different unique, individual instance mask. Image 822 illustrates multiple masks applied to one or more cytoplasms, with individual colors representing a different, unique individual instance mask. Image 830 illustrates a semantic mask, which is the union of all instance masks, applied to one or more nuclei. Image 832 illustrates a semantic mask applied to one or more cytoplasms. FIG. 20B further illustrates the use of mask categories. As shown in FIG. 20B, an individual instance mask can be applied to cells undergoing M phase, an individual instance mask can be applied to cells undergoing G1 phase, an individual instance mask can be applied to cells undergoing Early S phase, an individual instance mask can be applied to cells undergoing Middle S phase, an individual instance mask can be applied to cells undergoing Late S phase and/or an individual instance mask can be applied to cells undergoing G2 phase.

FIG. 9 illustrates an example computer-implemented environment 900 where an imaging system 910 can interact with a computing architecture to perform the various algorithms described herein. As shown in FIG. 9, the imaging system 910 can interface with one or more clients 950 (e.g., clients 702 via a web application having a graphical user interface such). The one or more clients 950 can interface with one or more servers 920 accessible through the network(s) 930. The one or more clients 950 can host a frame grabber that captures images from a camera (e.g., movies 612). Those images can be temporarily stored on the one or more clients 950 and periodically transferred to the one or more servers 920 for remote storage via network 930. The one or more servers 920 can also contain or have access to one or more data stores 940 for storing data collected and/or extracted from a sample by imaging system 910. In some variations, the network 930 may include or interface with one or more network storage arrays 960 for storing data such as the captured images (e.g., movies 612).

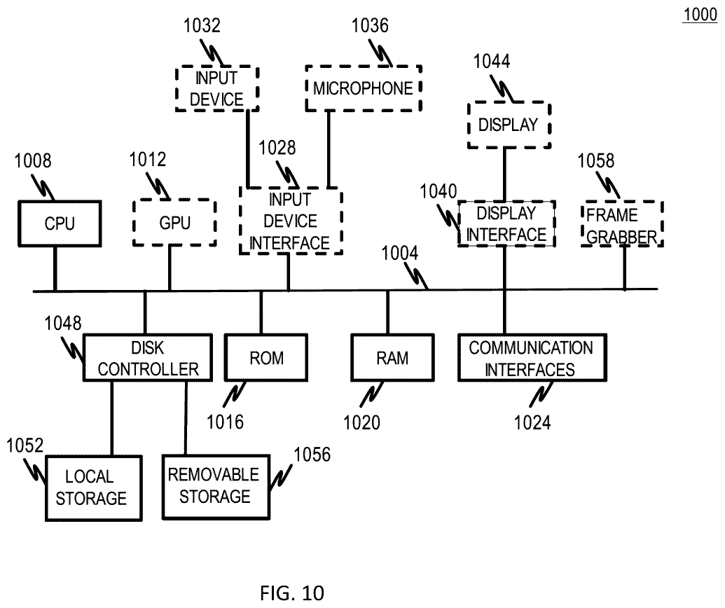

FIG. 10 is a diagram 1000 illustrating a sample computing device architecture for implementing various aspects described herein. In some variations, the sample computing device architecture can be that of client(s) 950 and/or of server(s) 920 and some components described in relation to diagram 1000 may be optional for the client(s) 950 and/or servers(s) 920. A bus 1004 can serve as the information highway interconnecting the other illustrated components of the hardware. A processing system 1008 labeled CPU (central processing unit) (e.g., one or more computer processors/data processors at a given computer or at multiple computers), can perform calculations and logic operations required to execute a program. Optionally or additionally, a processing system 1012 labeled GPU (graphics processing unit) (e.g., one or more computer processors/data processors at a given computer or at multiple computers), can perform calculations and logic operations required to execute a program. A non-transitory processor-readable storage medium, such as read only memory (ROM) 1016 and random access memory (RAM) 1020, can be in communication with the processing system 1008 and/or processing system 1012 and can include one or more programming instructions for the operations specified here. Optionally, program instructions can be stored on a non-transitory computer-readable storage medium such as a magnetic disk, optical disk, recordable memory device, flash memory, solid state drive or other physical storage medium.

In one example, a disk controller 1048 can interface with one or more optional removable storage 1056 or local storage 1052 to the system bus 1004. The removable storage 1056 can be external or internal disk drives, or solid state drives, or external hard drives. The local storage 1052 can be internal hard drives and/or memory. As indicated previously, these various examples of removable storage 1056, local storage 1052, and disk controllers 1048 are optional devices. The system bus 1004 can also include at least one communications interface 1024 to allow for communication with external devices either physically connected to the computing system or available externally through a wired or wireless network such as cloud storage and remote services. In some cases, the at least one communications interface 1024 includes or otherwise comprises a network interface.

In some variations, such as for client(s) 950, to provide for interaction with a user, the subject matter described herein can be implemented on a computing device having a display device 1044 (e.g., LCD (liquid crystal display) or LED (light-emitting diode) monitor) for displaying information obtained from the bus 1004 via a display interface 1040 to the user and an input device 1032 such as keyboard and/or a pointing device (e.g., a mouse or a trackball) and/or a touchscreen by which the user can provide input to the computer. Other kinds of input devices 1032 can be used to provide for interaction with a user as well; for example, feedback provided to the user can be any form of sensory feedback (e.g., visual feedback, auditory feedback by way of a microphone 1036, or tactile feedback); and input from the user can be received in any form, including acoustic, speech, or tactile input. The input device 1032 and the microphone 1036 can be coupled to and convey information via the bus 1004 by way of an input device interface 1028. By way of example, input device 1032 may be an imaging system 910 configured with abilities to capture a sequence of images as described herein. A frame grabber 1058 can capture or grab individual frames from analog or digital data encapsulating the sequence of images obtained from the bus 1004. Frame grabber 1058 may include memory that can store individual or multiple frames. Frame grabber 1058 can also provide individual or multiple frames to bus 1004 for further storage on, for example, local storage 1052 and/or removable storage 1056. Other computing devices, such as dedicated servers, can omit one or more of the components described in connection with FIG. 10.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

### 4. SPECIFIC OLS HTSMT APPLICATIONS

Many, and perhaps most, pathways that regulate the fundamental biochemistry of cells depend upon the interaction of protein sensors with protein effectors that engage transiently to trigger a change in cell physiology. Although the fundamentals of this process have long been appreciated, biochemical investigation of these protein interactions has typically required in vitro reconstitution or has been interrogated through pull-down assays after cell permeabilization. The htSMT workflow described herein provide a means of visualizing protein movement in large numbers of live cells, and under circumstances where the effect of added compositions, e.g., small molecule inhibitors, can be assessed quantitatively.

With reference to FIG. 1, aspects of the OLS htSMT workflows of the present disclosure include, but are not limited to, (i) sample preparation including reagent handling, (ii) image acquisition using imaging of the samples to generate a series of images and/or videos, (iii) image analysis through processing of these images and video, (iv) storage of information, and (v) provision of insights using the stored information including biological interpretation. With respect to the biological interpretations, the htSMT workflows described herein offer the ability to provide specific insights, as outlined below, depending on the particular workflow employed, e.g., (i) OLS htSMT Screening; (ii) OLS htSMT Binding; and/or (iii) OLS KineticSMT.

In certain embodiments, the workflows of the present disclosure can comprise detecting the fluorescence from a plurality of the target fluorescent proteins in a field of view of the sample plane, where the field of view (e.g., detected field of view) has a size of about 150 μm to about 250 μm in a first dimension by about 100 μm to about 210 μm in a second dimension. In certain embodiments, the FOV, e.g., detected FOV, can have a size of about 150 μm to about 250 μm in a first dimension by about 100 μm to about 210 μm in a second dimension. In certain embodiments, the FOV, e.g., detected FOV, can have a size of about 200 μm to about 250 μm in a first dimension by about 150 μm to about 210 μm in a second dimension or the FOV, e.g., detected FOV, can have a size of about 225 μm to about 250 μm in a first dimension by about 175 μm to about 210 μm in a second dimension. For example, but not by way of limitation, the FOV, e.g., detected FOV, can have a size of about 250 μm in a first dimension by about 190 μm in a second dimension, e.g., as disclosed in Example 1.

In certain embodiments, a certain percentage of an FOV, e.g., a detected FOV, achieves sufficient laser illumination for tracking protein movement. For example, but not by way of limitation, at least 75% of the FOV, at least 80% of the FOV, at least 85% of the FOV, at least 90% of the FOV, at least 95% of the FOV, at least 96% of the FOV, at least 97% of the FOV, at least 98% of the FOV, at least 99% of the FOV or 100% of the FOV achieves sufficient laser illumination for tracking protein movement. In certain embodiments, at least 75% of the FOV, e.g., detected FOV, achieves sufficient laser illumination for tracking protein movement. In certain embodiments, at least 80% of the FOV, e.g., detected FOV, achieves sufficient laser illumination for tracking protein movement. In certain embodiments, at least 85% of the FOV, e.g., detected FOV, achieves sufficient laser illumination for tracking protein movement. In certain embodiments, at least 90% of the FOV, e.g., detected FOV, achieves sufficient laser illumination for tracking protein movement. In certain embodiments, at least 95% of the FOV, e.g., detected FOV, achieves sufficient laser illumination for tracking protein movement. In certain embodiments, at least 96% of the FOV, e.g., detected FOV, achieves sufficient laser illumination for tracking protein movement. In certain embodiments, at least 97% of the FOV, e.g., detected FOV, achieves sufficient laser illumination for tracking protein movement. In certain embodiments, at least 98% of the FOV, e.g., detected FOV, achieves sufficient laser illumination for tracking protein movement. In certain embodiments, at least 99% of the FOV, e.g., detected FOV, achieves sufficient laser illumination for tracking protein movement. In certain embodiments, 100% of the FOV, e.g., detected FOV, achieves sufficient laser illumination for tracking protein movement. In certain embodiments, a percentage equal to or greater than about 75% of the FOV achieves sufficient laser illumination for tracking protein movement, e.g., a percentage equal to or greater than about 80% of the FOV, a percentage equal to or greater than about 85% of the FOV, a percentage equal to or greater than about 90% of the FOV, a percentage equal to or greater than about 95% of the FOV, a percentage equal to or greater than about 96% of the FOV, a percentage equal to or greater than about 97% of the FOV, a percentage equal to or greater than about 98% of the FOV or a percentage equal to or greater than about 99% of the FOV provides sufficient laser illumination for tracking protein movement. In certain embodiments, a percentage equal to or greater than about 90% of the FOV achieves sufficient laser illumination for tracking protein movement. In certain embodiments, a percentage equal to or greater than about 95% of the FOV achieves sufficient laser illumination for tracking protein movement.

In certain embodiments, the workflows of the present disclosure comprise detecting the field of view with a frame rate up to about 2000 Hz. In certain embodiments, the workflows of the present disclosure comprise detecting the field of view with a frame rate of about 100 Hz or higher, about 200 Hz or higher, about 400 Hz or higher, about 600 Hz or higher, about 800 Hz or higher, about 1000 Hz or higher, about 1200 Hz or higher, about 1400 Hz or higher, about 1600 Hz or higher or about 1800 Hz or higher. In certain embodiments, the workflows of the present disclosure comprise detecting the field of view with a frame rate up to about 1200 Hz. In certain embodiments, the workflows of the present disclosure comprise detecting the field of view with a frame rate up to about 1400 Hz. In certain embodiments, the workflows of the present disclosure comprise detecting the field of view with a frame rate up to about 1600 Hz. In certain embodiments, the workflows of the present disclosure comprise detecting the field of view with a frame rate up to about 1800 Hz.

In certain embodiments, the workflows of the present disclosure comprise illuminating the field of view using a 0.1 to 1 msec stroboscopic laser pulse. In certain embodiments, the workflows of the present disclosure comprise illuminating the field of view using a stroboscopic laser pulse of about 0.2 to about 1 msec, of about 0.3 to about 1 msec, of about 0.4 to about 1 msec, of about 0.1 to about 0.9 msec, of about 0.1 to about 0.8 msec, of about 0.1 to about 0.7 msec, of about 0.1 to about 0.6 msec, of about 0.1 to about 0.5 msec, of about 0.1 to about 0.4 msec, of about 0.2 to about 0.6 msec, of about 0.2 to about 0.5 msec, of about 0.2 to about 0.4 msec or of about 0.3 to about 0.5 msec. In certain embodiments, the workflows of the present disclosure comprise illuminating the field of view using a stroboscopic laser pulse of about 0.1 to about 0.6 msec. In certain embodiments, the workflows of the present disclosure comprise illuminating the field of view using a stroboscopic laser pulse of about 0.1 to about 0.5 msec. In certain embodiments, the workflows of the present disclosure comprise illuminating the field of view using a stroboscopic laser pulse of about 0.2 to about 0.4 msec. In certain embodiments, the workflows of the present disclosure comprise illuminating the field of view using a stroboscopic laser pulse of about 0.2 msec.

In certain embodiments, the workflows of the present disclosure comprise illuminating a field of view in a sample plane disposed within the sample with a light beam to cause fluorescence by a subset of the fluorescent target proteins in the live cells to image a plurality of molecular trajectories. In certain embodiments, up to about 1,000,000 molecular trajectories in a single detected field of view can be imaged, e.g., up to about 900,000, up to about 800,000, up to about 700,000, up to about 600,000, up to about 500,000, up to about 400,000, up to about 300,000, up to about 200,000 or up to about 100,000. In certain embodiments, the number of trajectories imaged in a single detected field of view can be from about 30,000 to about 1,000,000, e.g., about 30,000 to about 250,000. For example, but not by way of limitation, the number of trajectories imaged in a single detected field of view can be from about 50,000 to about 200,000, from about 100,000 to about 200,000, from about 100,000 to about 500,000 or from about 100,000 to about 150,000. In certain embodiments, the number of trajectories imaged in a single detected field of view can be up to about 1,000,000. In certain embodiments, the number of trajectories imaged in a single detected field of view can be from about 100,000 to about 1,000,000. In certain embodiments, the number of trajectories imaged in a single detected field of view can be from about 200,000 to about 1,000,000. In certain embodiments, the number of trajectories imaged in a single detected field of view can be from about 100,000 to about 500,000. In certain embodiments, the number of trajectories imaged in a single detected field of view can be from about 200,000 to about 500,000.

In certain embodiments, a field of view can include a plurality of cells. In certain embodiments, the number of cells imaged in a field of view is related to the size of the cells being imaged. For example, but not by way of limitation, the smaller the size of the cell, the greater the number of cells that can be imaged in a field of view. In certain embodiments, depending on the size of the cell being imaged, a field of view can include about 30 to about 200 live cells, e.g., can include about 30 to about 80 cells or about 50 to about 80 cells. In certain embodiments, depending on the size of the cell being imaged, a field of view can include up to about 80 live cells, e.g., mammalian cells. In certain embodiments, for U2OS cells the range is about 30 to about 40 cells per field of view, while for HCT116 cells the range is about 50 to about 80 cells per field of view given their differences in area. In certain embodiments, a field of view can include 30 to about 80 live cells, e.g., mammalian cells. In certain embodiments, a field of view can include 50 to about 80 live cells, e.g., mammalian cells. In certain embodiments, a field of view can include 55 to about 80 live cells, e.g., mammalian cells. In certain embodiments, a field of view can include 60 to about 80 live cells, e.g., mammalian cells.

In certain embodiments, the workflows of the present disclosure can include analyzing a subset (e.g., a subpopulation) of the cells present within the field of view, e.g., analyzing and/or tracking the trajectories of the fluorescent target proteins in a subset (e.g., a subpopulation) of the cells present within the field of view. For example, but not by way of limitation, workflows of the present disclosure can include analyzing about 1% to about 99% of the cells present within the field of view, e.g., about 1% to about 50% of the cells present within the field of view.

In certain embodiments, the workflows of the present disclosure can include illuminating a field of view in a sample plane disposed within the sample with a light beam to cause fluorescence a plurality of fluorescent target proteins in the live cells. In certain embodiments, the plurality of fluorescent target proteins can include from about 1,000 to about 1,000,000 fluorescent target proteins, e.g., about 10,000 to about 1,000,000 or about 100,000 to about 1,000,000.

In certain embodiments, the workflows of the present disclosure can include detecting the fluorescence from the plurality of fluorescent target proteins in a field of view of the sample plane at a rate of more than about 100,000 detected field of views per day. For example, but not by way of limitation, a rate of about 100,000 to about 1,000,000 detected field of views per day. In certain embodiments, the workflows of the present disclosure can include detecting the fluorescence from the plurality of fluorescent target proteins in a field of view of the sample plane a rate of about 100,000 to about 500,000 detected field of views per day.

In certain embodiments, the workflows of the present disclosure can include determining a change in the movement of the fluorescently labeled target protein in the presence of the compound. For example, but not by way of limitation, the average change in movement of the fluorescent target protein in the presence of a compound is about 1% to about 5% or to about 10% relative to baseline movements in the absence the compound. In certain embodiments, the average change in movement of the fluorescent target protein in the presence of a compound is about 1% to about 5%. In certain embodiments, the average change in movement of the fluorescent target protein in the presence of a compound is about 1% to about 10%.

In certain embodiments, exemplary OLS htSMT workflows comprise individual strategies described above as well as combinations of these strategies where two or more of the strategic requirements are combined.

4.1 OLS htSMT Screening

In certain implementations of the OLS htSMT workflows described herein, the systems and methods are adapted to interrogate the ability of one or more compositions, e.g., “test” compounds, to impact the SMT profile associated with a labeled protein. For example, such htSMT workflow will screen for changes in the SMT profile, e.g., either an increase or a decrease in movement of the protein of interest, in the presence of the composition relative to that SMT profile in the absence of the composition. It will be appreciated that higher-order comparisons can also be made with where compounds are multiplexed, including where multiple proteins are fluorescent. Moreover, as outlined above, the htSMT screening strategies described herein are equally applicable to screening of the SMT profiles associated with fluorescent compounds, e.g., compounds that are naturally fluorescent or those that have been modified to fluoresce or are linked to a fluorophore.

Underlying such htSMT screening strategies is the ability of the htSMT workflows described herein to extract accurate molecular trajectories at scale. Exemplary OLS htSMT workflows comprise the following individual strategies as well as combinations of the following strategies where two or more of the strategic requirements are combined. For example, but not by way of limitation, the workflows of the present disclosure comprise both illuminating a field of view in a sample plane disposed within the sample with a light beam to cause fluorescence by a subset of the fluorescent target proteins in the live cells, wherein the subset of the fluorescent target proteins are present in about 30 to about 80 live cells illuminated in the field of view of the sample plane as well as illuminating a field of view in a sample plane disposed within the sample with a light beam to cause fluorescence by a subset of the fluorescent target proteins in the live cells, wherein the subset of the fluorescent target proteins comprises a range of about 1000 to about 1,000,000 proteins. Similarly, illuminating sample planes to illuminate about 30 to about 80 live cells per FOV and/or causing the fluorescence of about 1000 to about 1,000,000 proteins can be combined with any of the other strategic requirements disclosed herein, e.g., determining the average change in movement of the fluorescent target protein in the presence of the compound is about 1% to about 5% or to about 10% relative to baseline movements in the absence the compound, detecting the fluorescence from a plurality of the fluorescent target proteins in a field of view of the sample plane at a rate of about 10,000 to about 18,000 per day per system and achieving, based on a single field of view, a z-factor of >0.5.

In certain implementations of the OLS htSMT screening workflows described herein, the workflow can comprise identifying a biological interaction between a compound and a fluorescent target protein in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample, wherein said tracking comprises: (i) illuminating a field of view in a sample plane disposed within the sample with a light beam to cause fluorescence by a subset of the fluorescent target proteins in the live cells, wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells given their differences in area; (ii) detecting the fluorescence from a plurality of the fluorescent target proteins in the field of view of the sample plane via a detector device; and (c) determining a change in the movement of the fluorescent target protein in the presence of the compound; wherein a change in the movement of the fluorescent target protein in the presence of the compound relative to the movement of the fluorescent target protein in the absence of the compound, e.g., when addition of the compound has been substituted by a control, such as, but not limited to, DMSO, identifies a biological interaction between the compound and the fluorescent target protein.

In certain implementations of the OLS htSMT screening workflows described herein, the workflow can comprise identifying a biological interaction between a compound and a fluorescent target protein in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample, wherein said tracking comprises: (i) illuminating a field of view in a sample plane disposed within the sample with a light beam to cause fluorescence by a subset of the fluorescent target proteins in the live cells, wherein the subset of the fluorescent target proteins comprises a range of about 1000 to about 1,000,000 proteins, wherein the number of proteins within the subset depend on the expression level of the protein of interest as well as the dye concentration being deemed adequate to label a subset of the proteins for robust SMT, both of which can be calculated and/or configured by one of skill in the art based on the disclosure of the instant application; (ii) detecting the fluorescence from a plurality of the fluorescent target proteins in a field of view of the sample plane via a detector device; and (c) determining a change in the movement of the fluorescent target protein in the presence of the compound; wherein a change in the movement of the fluorescent target protein in the presence of the compound relative to the movement of the fluorescent target protein in the absence of the compound identifies a biological interaction between the compound and the fluorescent target protein.

In certain implementations of the OLS htSMT screening workflows described herein, the workflow can comprise identifying a biological interaction between a compound and a fluorescent target protein in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample, wherein said tracking comprises: (i) illuminating a field of view in a sample plane disposed within the sample with a light beam to cause fluorescence by a subset of the fluorescent target proteins in the live cells; (ii) detecting the fluorescence from a plurality of the fluorescent target proteins in a field of view of the sample plane via a detector device; and (c) determining a change in the movement of the fluorescent target protein in the presence of the compound, wherein the average change in movement of the fluorescent target protein in the presence of the compound is about 1% to about 5% or to about 10% relative to baseline movements in the absence of the compound; wherein a change in the movement of the fluorescent target protein in the presence of the compound relative to the movement of the fluorescent target protein in the absence of the compound identifies a biological interaction between the compound and the fluorescent target protein.

In certain implementations of the OLS htSMT screening workflows described herein, the workflow can comprise identifying a biological interaction between a compound and a fluorescent target protein in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample, wherein said tracking comprises: (i) illuminating a field of view in a sample plane disposed within the sample with a light beam to cause fluorescence by a subset of the fluorescent target proteins in the live cells; (ii) detecting the fluorescence from a plurality of the fluorescent target proteins in a field of view of the sample plane via a detector device; (iii) wherein said tracking comprises detecting the fluorescence from a plurality of the fluorescent target proteins in a field of view of the sample plane at a rate of about 10,000 to about 18,000 per day per system; and (c) determining a change in the movement of the fluorescent target protein in the presence of the compound; wherein a change in the movement of the fluorescent target protein in the presence of the compound relative to the movement of the fluorescent target protein in the absence of the compound identifies a biological interaction between the compound and the fluorescent target protein.

In certain implementations of the OLS htSMT screening workflows described herein, the workflow can comprise identifying a biological interaction between a compound and a fluorescent target protein in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample, wherein said tracking comprises: (i) illuminating a field of view in a sample plane disposed within the sample with a light beam to cause fluorescence by a subset of the fluorescent target proteins in the live cells; (ii) detecting the fluorescence from a plurality of the fluorescent target proteins in a field of view of the sample plane via a detector device; and (iii) achieve, based on a single field of view, a z-factor of >0.5; and (c) determining a change in the movement of the fluorescent target protein in the presence of the compound; wherein a change in the movement of the fluorescent target protein in the presence of the compound relative to the movement of the fluorescent target protein in the absence of the compound identifies a biological interaction between the compound and the fluorescent target protein.

In certain implementations of the OLS htSMT screening workflows described herein, the workflow can comprise determining a dose response of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample, wherein said tracking comprises: (i) illuminating a field of view in a sample plane disposed within the sample with a light beam to cause fluorescence by at least a subset of the fluorescent target proteins in the live cells, wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells, given their differences in area; (ii) detecting the fluorescence from one or more of the fluorescent target proteins in the sample plane via a detector device; and (c) determining a change in the movement of the fluorescent target protein in the presence of the compound (d) repeating steps (b)-(c) for each of the plurality of samples across the range of compound concentrations; wherein a change in the movement of the fluorescent target protein in the presence of the compound across the concentration range indicates the dose response of the compound.

In certain implementations of the OLS htSMT screening workflows described herein, the workflow can comprise determining a dose response of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample, wherein said tracking comprises: (i) illuminating a field of view in a sample plane disposed within the sample with a light beam to cause fluorescence by at least a subset of the fluorescent target proteins in the live cells, wherein the subset of the fluorescent target proteins comprises a range of about 1000 to about 1,000,000 proteins, wherein the number of proteins within the subset depend on the expression level of the protein of interest as well as the dye concentration being deemed adequate to label a subset protein for robust SMT, both of which can be calculated and/or configured by one of skill in the art based on the disclosure of the instant application; (ii) detecting the fluorescence from one or more of the fluorescent target proteins in the sample plane via a detector device; and (c) determining a change in the movement of the fluorescent target protein in the presence of the compound; (d) repeating steps (b)-(c) for each of the plurality of samples across the range of compound concentrations; wherein a change in the movement of the fluorescent target protein in the presence of the compound across the concentration range indicates the dose response of the compound.

In certain implementations of the OLS htSMT screening workflows described herein, the workflow can comprise determining a dose response of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample, wherein said tracking comprises: (i) illuminating a field of view in a sample plane disposed within the sample with a light beam to cause fluorescence by at least a subset of the fluorescent target proteins in the live cells; (ii) detecting the fluorescence from one or more of the fluorescent target proteins in the sample plane via a detector device; and (c) determining a change in the movement of the fluorescent target protein in the presence of the compound, wherein the average change in movement of the fluorescent target protein in the presence of the compound is about 1% to about 5% or to about 10% relative to baseline movements in the absence of the compound; and (d) repeating steps (b)-(c) for each of the plurality of samples across the range of compound concentrations; wherein a change in the movement of the fluorescent target protein in the presence of the compound across the concentration range indicates the dose response of the compound.

In certain implementations of the OLS htSMT screening workflows described herein, the workflow can comprise determining a dose response of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample, wherein said tracking comprises: (i) illuminating a field of view in a sample plane disposed within the sample with a light beam to cause fluorescence by at least a subset of the fluorescent target proteins in the cells; (ii) detecting the fluorescence from one or more of the fluorescent target proteins in the sample plane via a detector device; (iii) wherein said tracking comprises detecting the fluorescence from a plurality of the fluorescent target proteins in a field of view of the sample plane at a rate of about 10,000 to about 18,000 per day per system; and (c) determining a change in the movement of the fluorescent target protein in the presence of the compound (d) repeating steps (b)-(c) for each of the plurality of samples across the range of compound concentrations; wherein a change in the movement of the fluorescent target protein in the presence of the compound across the concentration range indicates the dose response of the compound.

In certain implementations of the OLS htSMT screening workflows described herein, the workflow can comprise determining a dose response of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample, wherein said tracking comprises: (i) illuminating a field of view in a sample plane disposed within the sample with a light beam to cause fluorescence by at least a subset of the fluorescent target proteins in the cells; (ii) detecting the fluorescence from one or more of the fluorescent target proteins in the sample plane via a detector device; and (iii) achieve, based on a single field of view, a z-factor of >0.5; and (c) determining a change in the movement of the fluorescent target protein in the presence of the compound (d) repeating steps (b)-(c) for each of the plurality of samples across the range of compound concentrations; wherein a change in the movement of the fluorescent target protein in the presence of the compound across the concentration range indicates the dose response of the compound.

In certain implementations of the OLS htSMT screening workflows described herein, the workflow can comprise use of a microscopy system configured to identify a biological interaction between a compound and a fluorescent target protein in a live cell comprising: (a) a stage for supporting a sample, wherein the sample comprises a population of live cells, and where the live cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane, and wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells given their differences in area; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; and (ii) track the movement of individual fluorescent target proteins, (e) a memory; and (f) a processor in communication with the memory and the detector device, where the processor is capable of determining the change in the movement of the fluorescent target protein in the presence of the compound relative to the absence of the compound.

In certain implementations of the OLS htSMT screening workflows described herein, the workflow can comprise use of a microscopy system configured to identify a biological interaction between a compound and a fluorescent target protein in a live cell comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane; and wherein the subset of the fluorescent target proteins comprises a range of about 1000 to about 1,000,000 proteins, wherein the number of proteins within the subset depend on the expression level of the protein of interest as well as the dye concentration being deemed adequate to label a subset protein for robust SMT, both of which can be calculated and/or configured by one of skill in the art based on the disclosure of the instant application; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; and (ii) track the movement of individual fluorescent target proteins, (e) a memory; and (f) a processor in communication with the memory and the detector device, where the processor is capable of determining the change in the movement of the fluorescent target protein in the presence of the compound relative to the absence of the compound.

In certain implementations of the OLS htSMT screening workflows described herein, the workflow can comprise use of a microscopy system configured to identify a biological interaction between a compound and a fluorescent target protein in a live cell comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; and (ii) track the movement of individual fluorescent target proteins, wherein the average change in movement of the fluorescent target protein in the presence of the compound is about 1% to about 5% or to about 10% relative to baseline movements in the absence of the compound, (e) a memory; and (f) a processor in communication with the memory and the detector device, where the processor is capable of determining the change in the movement of the fluorescent target protein in the presence of the compound relative to the absence of the compound.

In certain implementations of the OLS htSMT screening workflows described herein, the workflow can comprise use of a microscopy system configured to identify a biological interaction between a compound and a fluorescent target protein in a live cell comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a plurality of the fluorescent target proteins in the sample are disposed in the sample plane; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; and (ii) track the movement of individual fluorescent target proteins, wherein said tracking comprises detecting the fluorescence from a plurality of the fluorescent target proteins in a field of view of the sample plane at a rate of about 10,000 to about 18,000 per day per system; (e) a memory; and (f) a processor in communication with the memory and the detector device, where the processor is capable of determining the change in the movement of the fluorescent target protein in the presence of the compound relative to the absence of the compound.

In certain implementations of the OLS htSMT screening workflows described herein, the workflow can comprise use of a microscopy system configured to identify a biological interaction between a compound and a fluorescent target protein in a live cell comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a plurality of the fluorescent target proteins in the sample are disposed in the sample plane; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; (ii) track the movement of individual fluorescent target proteins; and (iii) achieve, based on a single field of view, a z-factor of >0.5; (e) a memory; and (f) a processor in communication with the memory and the detector device, where the processor is capable of determining the change in the movement of the fluorescent target protein in the presence of the compound relative to the absence of the compound.

OLS htSMT Binding

In certain implementations of the OLS htSMT workflows described herein, the systems and methods are adapted to discriminate between recovery after exposure to a compound driven by an increase in residence time (decreasing k*_{off}), which would result in increasing f_{bound} seen in an htSMT screening assay. Importantly, neither FRAP nor htSMT can discriminate between recovery driven by an increase in residence time (decreasing k*_{off}) or increasing the rate of chromatin binding (increasing k*ₒₙ), either of which would result in increasing f_{bound}. By changing SMT acquisition conditions to reduce the illumination intensity and collect long frame exposures, only immobile proteins form spots. Under these imaging conditions, the distribution of track lengths provides a measure of relative residence times.

Exemplary OLS htSMT binding workflows comprise the following individual strategies as well as combinations of the following strategies where two or more of the strategic requirements are combined. For example, but not by way of limitation, the workflows of the present disclosure comprise both illuminating a field of view in a sample plane disposed within the sample with a light beam to cause fluorescence by a subset of the fluorescent target proteins in the live cells, wherein the subset of the fluorescent target proteins are present in about 30 to about 80 live cells illuminated in the field of view of the sample plane as well as illuminating a field of view in a sample plane disposed within the sample with a light beam to cause fluorescence by a subset of the fluorescent target proteins in the live cells, wherein the subset of the fluorescent target proteins comprises a range of about 1000 to about 1,000,000 proteins. Similarly, illuminating sample planes to illuminate about 30 to about 80 live cells per FOV and/or causing the fluorescence of about 1000 to about 1,000,000 proteins can be combined with any of the other strategic requirements disclosed herein, e.g., determining the average change in movement of the fluorescent target protein in the presence of the compound is about 1% to about 5% or to about 10% relative to baseline movements in the absence of the compound, e.g., when addition of the compound has been substituted by a control, such as, but not limited to, DMSO, detecting the fluorescence from a plurality of the fluorescent target proteins in a field of view of the sample plane at a rate of about 10,000 to about 18,000 per day per system and achieving, based on a single field of view, a z-factor of >0.5.

In certain implementations of the OLS htSMT binding workflows described herein, the workflow will comprise determining whether a compound that induces a change in binding of a fluorescent target protein in a live cell reduces the K_{off} of the fluorescent target protein comprising: (a) contacting a sample comprising a population of live cells with the compound, where the live cells comprise the fluorescent target protein; (b) tracking the movement of individual fluorescent target proteins in a plurality of the cells in the sample, wherein said tracking comprises: (i) illuminating a field of view in a sample plane disposed within the sample with a light beam to cause fluorescence by at least a subset of the fluorescent target proteins in the live cells, wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells given their differences in area; (ii) detecting the fluorescence from one or more of the fluorescent target proteins in the sample plane via a detector device where the method is adapted to selectively detect localized fluorescence; and (c) determining a change in the movement of the fluorescent target protein in the presence of the compound; wherein an increase in the signal detected from the fluorescent target protein in the presence of the compound relative to the signal of the fluorescent target protein in the absence of the compound indicates that the compound induces a reduction in the K_{off} of the fluorescent target protein.

In certain implementations of the OLS htSMT binding workflows described herein, the workflow can comprise determining whether a compound that induces a change in binding of a fluorescent target protein in a live cell reduces the K_{off} of the fluorescent target protein comprising: (a) contacting a sample comprising a population of live cells with the compound, where the live cells comprise the fluorescent target protein; (b) tracking the movement of individual fluorescent target proteins in a plurality of the cells in the sample, wherein said tracking comprises: (i) illuminating a field of view in a sample plane disposed within the sample with a light beam to cause fluorescence by at least a subset of the fluorescent target proteins in the live cells, wherein the subset of the fluorescent target proteins comprises a range of about 1000 to about 1,000,000 proteins, wherein the number of proteins within the subset depend on the expression level of the protein of interest as well as the dye concentration being deemed adequate to label a subset protein for robust SMT, both of which can be calculated and/or configured by one of skill in the art based on the disclosure of the instant application; (ii) detecting the fluorescence from one or more of the fluorescent target proteins in the sample plane via a detector device where the method is adapted to selectively detect localized fluorescence; and (c) determining a change in the movement of the fluorescent target protein in the presence of the compound; wherein an increase in the signal detected from the fluorescent target protein in the presence of the compound relative to the signal of the fluorescent target protein in the absence of the compound indicates that the compound induces a reduction in the K_{off} of the fluorescent target protein.

In certain implementations of the OLS htSMT binding workflows described herein, the workflow can comprise determining whether a compound that induces a change in binding of a fluorescent target protein in a live cell reduces the K_{off} of the fluorescent target protein comprising: (a) contacting a sample comprising a population of live cells with the compound, where the live cells comprise the fluorescent target protein; (b) tracking the movement of individual fluorescent target proteins in a plurality of the cells in the sample, wherein said tracking comprises: (i) illuminating a field of view in a sample plane disposed within the sample with a light beam to cause fluorescence by at least a subset of the fluorescent target proteins in the live cells; (ii) detecting the fluorescence from one or more of the fluorescent target proteins in the sample plane via a detector device where the method is adapted to selectively detect localized fluorescence; and (c) determining a change in the movement of the fluorescent target protein in the presence of the compound, wherein the average change in movement of the fluorescent target protein in the presence of the compound is about 1% to about 5% or to about 10% relative to baseline movements in the absence of the compound; wherein an increase in the signal detected from the fluorescent target protein in the presence of the compound relative to the signal of the fluorescent target protein in the absence of the compound indicates that the compound induces a reduction in the K_{off} of the fluorescent target protein.

In certain implementations of the OLS htSMT binding workflows described herein, the workflow can comprise determining whether a compound that induces a change in binding of a fluorescent target protein in a live cell reduces the K_{off} of the fluorescent target protein comprising: (a) contacting a sample comprising a population of live cells with the compound, where the live cells comprise the fluorescent target protein; (b) tracking the movement of individual fluorescent target proteins in a plurality of the cells in the sample, wherein said tracking comprises: (i) illuminating a field of view in a sample plane disposed within the sample with a light beam to cause fluorescence by at least a subset of the fluorescent target proteins in the live cells; (ii) detecting the fluorescence from one or more of the fluorescent target proteins in the sample plane via a detector device where the method is adapted to selectively detect localized fluorescence; and (iii) achieving, based on a single field of view, a z-factor of >0.5; and (c) determining a change in the movement of the fluorescent target protein in the presence of the compound; wherein an increase in the signal detected from the fluorescent target protein in the presence of the compound relative to the signal of the fluorescent target protein in the absence of the compound indicates that the compound induces a reduction in the K_{off} of the fluorescent target protein

In certain implementations of the OLS htSMT binding workflows described herein, the workflow can comprise determining a dose of a compound that induces a change in binding of a fluorescent target protein in a live cell by determining whether the compound reduces the K_{off} of the fluorescent target protein comprising: (a) contacting a sample comprising a population of live cells with the compound, where the live cells comprise the fluorescent target protein; (b) tracking the movement of individual fluorescent target proteins in a plurality of the cells in the sample, wherein said tracking comprises: (i) illuminating a field of view in a sample plane disposed within the sample with a light beam to cause fluorescence by at least a subset of the fluorescent target proteins in the live cells, wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells given their differences in area; (ii) detecting the fluorescence from one or more of the fluorescent target proteins in the sample plane via a detector device where the method is adapted to selectively detect localized fluorescence and (c) determining a change in the movement of the fluorescent target protein in the presence of the compound; wherein an increase in the signal detected from the fluorescent target protein in the presence of the compound relative to the signal of the fluorescent target protein in the absence of the compound indicates that the compound induces a reduction in the K_{off} of the fluorescent target protein and indicates an increase in dose due to enhanced residence time leading to reduced drug metabolism.

In certain implementations of the OLS htSMT binding workflows described herein, the workflow can comprise determining a dose of a compound that induces a change in binding of a fluorescent target protein in a live cell by determining whether the compound reduces the K_{off} of the fluorescent target protein comprising: (a) contacting a sample comprising a population of live cells with the compound, where the live cells comprise the fluorescent target protein; (b) tracking the movement of individual fluorescent target proteins in a plurality of the cells in the sample, wherein said tracking comprises: (i) illuminating a field of view in a sample plane disposed within the sample with a light beam to cause fluorescence by at least a subset of the fluorescent target proteins in the live cells, wherein the subset of the fluorescent target proteins comprises a range of about 1000 to about 1,000,000 proteins, wherein the number of proteins within the subset depend on the expression level of the protein of interest as well as the dye concentration being deemed adequate to label a subset protein for robust SMT, both of which can be calculated and/or configured by one of skill in the art based on the disclosure of the instant application; (ii) detecting the fluorescence from one or more of the fluorescent target proteins in the sample plane via a detector device where the method is adapted to selectively detect localized fluorescence; and (c) determining a change in the movement of the fluorescent target protein in the presence of the compound; wherein an increase in the signal detected from the fluorescent target protein in the presence of the compound relative to the signal of the fluorescent target protein in the absence of the compound indicates that the compound induces a reduction in the K_{off} of the fluorescent target protein and, in certain instances, an increase in dose due to enhanced residence time leading to reduced drug metabolism.

In certain implementations of the OLS htSMT binding workflows described herein, the workflow can comprise determining a dose of a compound that induces a change in binding of a fluorescent target protein in a live cell by determining whether the compound reduces the K_{off} of the fluorescent target protein comprising: (a) contacting a sample comprising a population of live cells with the compound, where the live cells comprise the fluorescent target protein; (b) tracking the movement of individual fluorescent target proteins in a plurality of the cells in the sample, wherein said tracking comprises: (i) illuminating a field of view in a sample plane disposed within the sample with a light beam to cause fluorescence by at least a subset of the fluorescent target proteins in the live cells; (ii) detecting the fluorescence from one or more of the fluorescent target proteins in the sample plane via a detector device where the method is adapted to selectively detect localized fluorescence; and (c) determining a change in the movement of the fluorescent target protein in the presence of the compound; wherein the average change in movement of the fluorescent target protein in the presence of the compound is about 1% to about 5% or to about 10% relative to baseline movements in the absence of the compound; wherein an increase in the signal detected from the fluorescent target protein in the presence of the compound relative to the signal of the fluorescent target protein in the absence of the compound indicates that the compound induces a reduction in the K_{off} of the fluorescent target protein indicates an increase in dose due to enhanced residence time leading to reduced drug metabolism.

In certain implementations of the OLS htSMT binding workflows described herein, the workflow can comprise determining a dose of a compound that induces a change in binding of a fluorescent target protein in a live cell by determining whether the compound reduces the K_{off} of the fluorescent target protein comprising: (a) contacting a sample comprising a population of live cells with the compound, where the live cells comprise the fluorescent target protein; (b) tracking the movement of individual fluorescent target proteins in a plurality of the cells in the sample, wherein said tracking comprises: (i) illuminating a field of view in a sample plane disposed within the sample with a light beam to cause fluorescence by at least a subset of the fluorescent target proteins in the live cells; (ii) detecting the fluorescence from one or more of the fluorescent target proteins in the sample plane via a detector device where the method is adapted to selectively detect localized fluorescence; and (iii) achieving, based on a single field of view, a z-factor of >0.5; and (c) determining a change in the movement of the fluorescent target protein in the presence of the compound; wherein either: an increase in the signal detected from the fluorescent target protein in the presence of the compound relative to the signal of the fluorescent target protein in the absence of the compound indicates that the compound induces a reduction in the K_{off} of the fluorescent target protein and, in certain instances, an increase in dose due to enhanced residence time leading to reduced drug metabolism.

In certain implementations of the OLS htSMT binding workflows described herein, the workflow can comprise the use of a microscopy system configured to determine whether a compound that induces a change in binding of a fluorescent target protein in a cell reduces the K_{off} of the fluorescently labeled target comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane, and wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells given their differences in area; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; and (ii) track the movement of individual fluorescent target proteins, wherein the tracking is adapted to selectively detect localized fluorescence relative to dynamic fluorescence; (e) a memory; and (f) a processor in communication with the memory and the detector device, where the processor is capable of determining the change in the movement of the fluorescent target protein in the presence of the compound relative to the absence of the compound.

In certain implementations of the OLS htSMT binding workflows described herein, the workflow can comprise the use of a microscopy system configured to determine whether a compound that induces a change in binding of a fluorescent target protein in a cell reduces the K_{off} of the fluorescently labeled target comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane, and wherein the subset of the fluorescent target proteins comprises a range of about 1000 to about 1,000,000 proteins, wherein the number of proteins within the subset depend on the expression level of the protein of interest as well as the dye concentration being deemed adequate to label a subset protein for robust SMT, both of which can be calculated and/or configured by one of skill in the art based on the disclosure of the instant application; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; and (ii) track the movement of individual fluorescent target proteins, wherein the tracking is adapted to selectively detect localized fluorescence relative to dynamic fluorescence; (e) a memory; and (f) a processor in communication with the memory and the detector device, where the processor is capable of determining the change in the movement of the fluorescent target protein in the presence of the compound relative to the absence of the compound.

In certain implementations of the OLS htSMT binding workflows described herein, the workflow can comprise the use of a microscopy system configured to determine whether a compound that induces a change in binding of a fluorescent target protein in a cell reduces the K_{off} of the fluorescently labeled target comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; and (ii) track the movement of individual fluorescent target proteins, wherein the tracking is adapted to selectively detect localized fluorescence relative to dynamic fluorescence, and wherein the average change in movement of the fluorescent target protein in the presence of the compound is about 1% to about 5% or to about 10% relative to baseline movements in the absence of the compound; (e) a memory; and (f) a processor in communication with the memory and the detector device, where the processor is capable of determining the change in the movement of the fluorescent target protein in the presence of the compound relative to the absence of the compound.

In certain implementations of the OLS htSMT binding workflows described herein, the workflow can comprise the use of a microscopy system configured to determine whether a compound that induces a change in binding of a fluorescent target protein in a cell reduces the K_{off} of the fluorescently labeled target comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins; and (ii) track the movement of individual fluorescent target proteins, wherein the tracking is adapted to selectively detect localized fluorescence relative to dynamic fluorescence; (iii) achieve, based on a single field of view, a z-factor of >0.5; (e) a memory; and (f) a processor in communication with the memory and the detector device, where the processor is capable of determining the change in the movement of the fluorescent target protein in the presence of the compound relative to the absence of the compound.

### 4.3 OLS KineticSMT

Since SMT can identify the rate of emergence of a biological interaction between a compound and a target, SMT can be used to distinguish direct versus indirect effects on target activity, among other parameters. Given the live cell setting of SMT, a data collection mode can be configured that allows for measurement of protein movements in set intervals after compound addition (kinetic SMT or kSMT) to determine the rate of emergence of a biological interaction between a compound and a target.

Exemplary OLS KineticSMT workflows comprise the following individual strategies as well as combinations of the following strategies where two or more of the strategic requirements are combined. For example, but not by way of limitation, the workflows of the present disclosure comprise both illuminating a field of view in a sample plane disposed within the sample with a light beam to cause fluorescence by a subset of the fluorescent target proteins in the live cells, wherein the subset of the fluorescent target proteins are present in about 30 to about 80 live cells illuminated in the field of view of the sample plane as well as illuminating a field of view in a sample plane disposed within the sample with a light beam to cause fluorescence by a subset of the fluorescent target proteins in the live cells, wherein the subset of the fluorescent target proteins comprises a range of about 1000 to about 1,000,000 proteins. Similarly, illuminating sample planes to illuminate about 30 to about 80 live cells per FOV and/or causing the fluorescence of about 1000 to about 1,000,000 proteins can be combined with any of the other strategic requirements disclosed herein, e.g., determining the average change in movement of the fluorescent target protein in the presence of the compound is about 1% to about 5% or to about 10% relative to baseline movements in the absence of the compound, e.g., when addition of the compound has been substituted by a control, such as, but not limited to, DMSO, detecting the fluorescence from a plurality of the fluorescent target proteins in a field of view of the sample plane at a rate of about 10,000 to about 18,000 per day per system and achieving, based on a single field of view, a z-factor of >0.5.

In certain implementations of the OLS Kinetic htSMT binding workflows described herein, the workflow can comprise determining the rate of emergence of a biological interaction between a compound and a target in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample at a plurality of time points, wherein said tracking comprises: (i) illuminating a field of view in a sample plane disposed within the sample with a light beam to cause fluorescence by a subset of the fluorescent target proteins in the live cells, wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells given their differences in area; (ii) detecting the fluorescence from a plurality of the fluorescent target proteins in the field of view of the sample plane via a detector device; and (c) determining a change in the movement of the fluorescent target protein in the presence of the compound; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise determining the rate of emergence of a biological interaction between a compound and a target in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample at a plurality of time points, wherein said tracking comprises: (i) illuminating a field of view in a sample plane disposed within the sample with a light beam to cause fluorescence by a subset of the fluorescent target proteins in the live cells, wherein the subset of the fluorescent target proteins comprises a range of about 1000 to about 1,000,000 proteins, wherein the number of proteins within the subset depend on the expression level of the protein of interest as well as the dye concentration being deemed adequate to label a subset protein for robust SMT, both of which can be calculated and/or configured by one of skill in the art based on the disclosure of the instant application; (ii) detecting the fluorescence from a plurality of the fluorescent target proteins in a field of view of the sample plane via a detector device; and (c) determining a change in the movement of the fluorescent target protein in the presence of the compound; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise determining the rate of emergence of a biological interaction between a compound and a target in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample at a plurality of time points, wherein said tracking comprises: (i) illuminating a field of view in a sample plane disposed within the sample with a light beam to cause fluorescence by a subset of the fluorescent target proteins in the live cells; (ii) detecting the fluorescence from a plurality of the fluorescent target proteins in a field of view of the sample plane via a detector device; and (c) determining a change in the movement of the fluorescent target protein in the presence of the compound, wherein the average change in movement of the fluorescent target protein in the presence of the compound is about 1% to about 5% or to about 10% relative to baseline movements in the absence of the compound; wherein the rate at which changes in the movement of the fluorescent target protein occur in the presence of the compound indicates t the rate of emergence of a biological interaction between a compound and a target.

In certain implementations of the OLS Kinetic htSMT binding workflows described herein, the workflow can comprise determining the rate of emergence of a biological interaction between a compound and a target between a direct and indirect biological interaction between a compound and a fluorescent target protein in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample at a plurality of time points, wherein said tracking comprises: (i) illuminating a field of view in a sample plane disposed within the sample with a light beam to cause fluorescence by a subset of the fluorescent target proteins in the live cells; (ii) detecting the fluorescence from a plurality of the fluorescent target proteins in a field of view of the sample plane via a detector device; (iii) wherein said tracking comprises detecting the fluorescence from a plurality of the fluorescent target proteins in a field of view of the sample plane at a rate of about 10,000 to about 18,000 per day per system; and (c) determining a change in the movement of the fluorescent target protein in the presence of the compound; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise determining the rate of emergence of a biological interaction between a compound and a target in a live cell comprising: (a) contacting a sample comprising a population of live cells with the compound, wherein the live cells comprise the fluorescent target protein; (b) tracking the movement of a plurality of individual fluorescent target proteins in a plurality of live cells in the sample at a plurality of time points, wherein said tracking comprises: (i) illuminating a field of view in a sample plane disposed within the sample with a light beam to cause fluorescence by a subset of the fluorescent target proteins in the live cells; (ii) detecting the fluorescence from a plurality of the fluorescent target proteins in the field of view of the sample plane via a detector device, wherein detection based on a single field of view is associated with a z-factor of >0.5; and (c) determining a change in the movement of the fluorescent target protein in the presence of the compound; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise determining a dose of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample at a plurality of time points, wherein said tracking comprises: (i) illuminating a field of view in a sample plane disposed within the sample with a light beam to cause fluorescence by at least a subset of the fluorescent target proteins in the live cells, wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells given their differences in area; (ii) detecting the fluorescence from one or more of the fluorescent target proteins in the sample plane via a detector device; and (c) determining the rate at which changes in the movement of the fluorescent target protein occur in the presence of the compound; (d) repeating steps (b)-(c) for each of the plurality of samples across the range of compound concentrations; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise determining a dose of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample at a plurality of time points, wherein said tracking comprises: (i) illuminating a field of view in a sample plane disposed within the sample with a light beam to cause fluorescence by at least a subset of the fluorescent target proteins in the live cells, wherein the subset of the fluorescent target proteins comprises a range of about 1000 to about 1,000,000 proteins, wherein the number of proteins within the subset depend on the expression level of the protein of interest as well as the dye concentration being deemed adequate to label a subset protein for robust SMT, both of which can be calculated and/or configured by one of skill in the art based on the disclosure of the instant application; (ii) detecting the fluorescence from one or more of the fluorescent target proteins in the sample plane via a detector device; and (c) determining the rate at which changes in the movement of the fluorescent target protein occur in the presence of the compound; (d) repeating steps (b)-(c) for each of the plurality of samples across the range of compound concentrations; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise determining a dose response of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample at a plurality of time points, wherein said tracking comprises: (i) illuminating a field of view in a sample plane disposed within the sample with a light beam to cause fluorescence by at least a subset of the fluorescent target proteins in the live cells; (ii) detecting the fluorescence from one or more of the fluorescent target proteins in the sample plane via a detector device; and (c) determining the rate at which changes in the movement of the fluorescent target protein occur in the presence of the compound, wherein the average change in movement of the fluorescent target protein in the presence of the compound is about 1% to about 5% or to about 10% relative to baseline movements in the absence of the compound; and (d) repeating steps (b)-(c) for each of the plurality of samples across the range of compound concentrations; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise determining a dose response of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample at a plurality of time points, wherein said tracking comprises: (i) illuminating a field of view in a sample plane disposed within the sample with a light beam to cause fluorescence by at least a subset of the fluorescent target proteins in the cells; (ii) detecting the fluorescence from one or more of the fluorescent target proteins in the sample plane via a detector device; (iii) wherein said tracking comprises detecting the fluorescence from a plurality of the fluorescent target proteins in a field of view of the sample plane at a rate of about 10,000 to about 18,000 per day per system; and (c) determining the rate at which changes in the movement of the fluorescent target protein occur in the presence of the compound; (d) repeating steps (b)-(c) for each of the plurality of samples across the range of compound concentrations; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise determining a dose of a compound that induces a change in the movement of a fluorescent target protein in a live cell comprising: (a) contacting a plurality of samples with the compound, (i) where each sample comprises a population of live cells, (ii) where the live cells comprise the fluorescent target protein, and (iii) where the plurality of samples are contacted with distinct concentrations of the compound across a range of compound concentrations; (b) tracking the movement of individual fluorescent target proteins in a plurality of live cells of a sample at a plurality of time points, wherein said tracking comprises: (i) illuminating a field of view in a sample plane disposed within the sample with a light beam to cause fluorescence by at least a subset of the fluorescent target proteins in the live cells; (ii) detecting the fluorescence from one or more of the fluorescent target proteins in the field of view in the sample plane via a detector device, wherein detection based on a single field of view is associated with a z-factor of >0.5; and (c) determining the rate at which changes in the movement of the fluorescent target protein occur in the presence of the compound; (d) repeating steps (b)-(c) for each of the plurality of samples across the range of compound concentrations; wherein the rate at which changes in the movement of the fluorescent target protein occur in the presence of the compound indicates the rate of emergence of a biological interaction between a compound and a target.

In certain implementations of the OLS Kinetic htSMT binding workflows described herein, the workflow can comprise use of a microscopy system configured to determine the rate of emergence of a biological interaction between a compound and a target in a live cell comprising: (a) a stage for supporting a sample, wherein the sample comprises a population of live cells, and where the live cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane, and wherein the subset of the fluorescent target proteins are present in about 30 to about 80 live cells illuminated in the field of view of the sample plane, depending on the specific cell type being used, e.g., for U2OS cells the range is about 30 to about 40 cells per FOV, while for HCT116 cells the range is about 50 to about 80 cells given their differences in area; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins at a plurality of time points; and (ii) track the movement of individual fluorescent target proteins, (e) a memory; and (f) a processor in communication with the memory and the detector device, where the processor is capable of determining the change in the movement of the fluorescent target protein in the presence of the compound relative to the absence of the compound.

In certain implementations of the OLS Kinetic htSMT binding workflows described herein, the workflow can comprise use of a microscopy system configured to determine the rate of emergence of a biological interaction between a compound and a target in a live cell comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane; and wherein the subset of the fluorescent target proteins comprises a range of about 1000 to about 1,000,000 proteins, wherein the number of proteins within the subset depend on the expression level of the protein of interest as well as the dye concentration being deemed adequate to label a subset protein for robust SMT, both of which can be calculated and/or configured by one of skill in the art based on the disclosure of the instant application; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins at a plurality of time points; and (ii) track the movement of individual fluorescent target proteins, (e) a memory; and (f) a processor in communication with the memory and the detector device, where the processor is capable of determining the change in the movement of the fluorescent target protein in the presence of the compound relative to the absence of the compound.

In certain implementations of the OLS Kinetic htSMT binding workflows described herein, the workflow can comprise use of a microscopy system configured to determine the rate of emergence of a biological interaction between a compound and a target in a live cell comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins at a plurality of time points; and (ii) track the movement of individual fluorescent target proteins, wherein the average change in movement of the fluorescent target protein in the presence of the compound is about 1% to about 5% or to about 10% relative to baseline movements in the absence of the compound, (e) a memory; and (f) a processor in communication with the memory and the detector device, where the processor is capable of determining the change in the movement of the fluorescent target protein in the presence of the compound relative to the absence of the compound.

In certain implementations of the OLS Kinetic htSMT binding workflows described herein, the workflow can comprise use of a microscopy system configured to determine the rate of emergence of a biological interaction between a compound and a target in a live cell comprising: (a) a stage for supporting a sample, wherein the sample comprises a population of cells, and where the cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a plurality of the fluorescent target proteins in the sample are disposed in the sample plane; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins at a plurality of time points; and (ii) track the movement of individual fluorescent target proteins, wherein said tracking comprises detecting the fluorescence from a plurality of the fluorescent target proteins in a field of view of the sample plane at a rate of about 10,000 to about 18,000 per day per system; (e) a memory; and (f) a processor in communication with the memory and the detector device, where the processor is capable of determining the change in the movement of the fluorescent target protein in the presence of the compound relative to the absence of the compound.

In certain implementations of the OLS Kinetic htSMT binding workflows described herein, the workflow can comprise use of a microscopy system configured to determine the rate of emergence of a biological interaction between a compound and a target in a live cell comprising: (a) a stage for supporting a sample, wherein the sample comprises a population of live cells, and where the live cells comprise the fluorescent target protein; (b) a light source for emitting a light beam capable of inducing a light-based response from a plurality of the fluorescent target proteins in the sample; (c) an objective for focusing the light beam on the sample in the sample plane, wherein a subset of the fluorescent target proteins in the sample are disposed in a field of view in the sample plane; (d) a detector device for monitoring the light-based response from the fluorescent target proteins in the presence of the compound, wherein the detector device is configured to: (i) block light received from sources other than the sample plane in which the fluorescent target proteins are disposed to thereby track the position of the fluorescent target proteins at a plurality of time points; and (ii) track the movement of individual fluorescent target proteins; and (iii) achieve, based on a single field of view, a z-factor of >0.5; (e) a memory; and (f) a processor in communication with the memory and the detector device, where the processor is capable of determining the change in the movement of the fluorescent target protein in the presence of the compound relative to the absence of the compound.

### 5. EXEMPLARY EMBODIMENTS

A. The present disclosure provides a method of identifying a biological interaction between a compound and a target fluorescent protein in a live cell comprising:
(a) contacting a sample comprising a population of live cells with the compound, wherein the live cells comprise target fluorescent protein;
(b) tracking the movement of a plurality of individual target fluorescent proteins in a plurality of live cells in the sample, wherein said tracking comprises:
   (i) illuminating a field of view in a sample plane disposed within the sample with a light beam to cause fluorescence by a subset of the target fluorescent proteins in the live cells; and
   (ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension; and
(c) determining a change in the movement of at a target fluorescent protein in the presence of the compound,
   wherein a change in the movement of the target fluorescent protein in the presence of the compound relative to the movement of the target fluorescent protein in the absence of the compound identifies a biological interaction between the compound and the target fluorescent protein.

B. The present disclosure provides a method of identifying a biological interaction between a compound and a target fluorescent protein in a live cell comprising:
(a) contacting a sample comprising a population of live cells with the compound, wherein the live cells comprise target fluorescent protein;
(b) tracking the movement of a plurality of individual target fluorescent protein in a plurality of live cells in the sample, wherein said tracking comprises:
   (i) illuminating a field of view in a sample plane disposed within the sample with a light beam to cause fluorescence by a subset of the target fluorescent proteins in the live cells, wherein the subset of the target fluorescent proteins produces up to about 1,000,000 molecular trajectories in a single detected field of view; and
   (ii) detecting the fluorescence from a plurality of the target fluorescent proteins in the detected field of view of the sample plane via a detector device, wherein the detected field of view has a size of about 150 μm to about 250 μm in a first dimension by about 100 μm to about 210 μm in a second dimension; and
(c) determining a change in the movement of a target fluorescent protein in the presence of the compound,
   wherein a change in the movement of the target fluorescent protein in the presence of the compound relative to the movement of the target fluorescent protein in the absence of the compound identifies a biological interaction between the compound and the target fluorescent protein.

C. The present disclosure provides a method of identifying a biological interaction between a compound and a target fluorescent protein in a live cell comprising:
(a) contacting a sample comprising a population of live cells with the compound, wherein the live cells comprise target fluorescent proteins;
(b) tracking the movement of a plurality of individual target fluorescent proteins in a plurality of live cells in the sample, wherein said tracking comprises:
   (i) illuminating a field of view in a sample plane disposed within the sample with a light beam to cause fluorescence by a subset of the target fluorescent proteins in the live cells, wherein the field of view is illuminated using a 0.1 to 1 msec stroboscopic laser pulse; and
   (ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension; and
(c) determining a change in the movement of the target fluorescent proteins in the presence of the compound,
   wherein a change in the movement of a target fluorescent protein in the presence of the compound relative to the movement of the target fluorescent protein in the absence of the compound identifies a biological interaction between the compound and the target fluorescent protein.

D. The present disclosure provides a method of identifying a biological interaction between a compound and a target fluorescent protein in a live cell comprising:
(a) contacting a sample comprising a population of live cells with the compound, wherein the live cells comprise target fluorescent proteins;
(b) tracking the movement of a plurality of individual target fluorescent proteins in a plurality of live cells in the sample, wherein said tracking comprises:
   (i) illuminating a field of view in a sample plane disposed within the sample with a light beam to cause fluorescence by a subset of the target fluorescent proteins in the live cells;
   (ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension; and
   (iii) detecting the fluorescence from a plurality of the target fluorescent proteins in the detected field of view of the sample plane at a rate of more than about 100,000 detected field of views per day; and
(c) determining a change in the movement of a target fluorescent protein in the presence of the compound,
   wherein a change in the movement of the target fluorescent proteins in the presence of the compound relative to the movement of target fluorescent protein in the absence of the compound identifies a biological interaction between the compound and the target fluorescent protein.

E. The present disclosure provides a method of identifying a biological interaction between a compound and a target fluorescent protein in a live cell comprising:
(a) contacting a sample comprising a population of live cells with the compound, wherein the live cells comprise the fluorescently labeled target protein;
(b) tracking the motion of a plurality of individual target fluorescent proteins in a plurality of live cells in the sample, wherein said tracking comprises:
   (i) illuminating a field of view in a sample plane disposed within the sample with a light beam to cause fluorescence by a subset of the target fluorescent proteins in the live cells;
   (ii) detecting the fluorescence from a plurality of the target fluorescent proteins in a detected field of view of the imaging plane via a detector device, wherein the detected field of view has a size of about 150 μm to about 250 μm in a first dimension by about 100 μm to about 210 μm in a second dimension; and
   (iii) achieve, based on a single field of view, a z-factor of >0.5; and
(c) determining a change in the movement of the fluorescently labeled target protein in the presence of the compound,
   wherein a change in the movement of the target fluorescent protein in the presence of the compound relative to the motion of the target fluorescent protein in the absence of the compound identifies a biological interaction between the compound and the target fluorescent protein.

F. The present disclosure provides a method of identifying a biological interaction between a compound and a target fluorescent protein in a live cell comprising:
(a) contacting a sample comprising a population of live cells with the compound, wherein the live cells comprise target fluorescent proteins;
(b) tracking the movement of a plurality of individual target fluorescent proteins in a plurality of live cells in the sample, wherein said tracking comprises:
   (i) illuminating a field of view in a sample plane disposed within the sample with a light beam to cause fluorescence by a subset of the target fluorescent proteins in the live cells; and
   (ii) detecting the fluorescence from a plurality of the target fluorescent proteins in a detected field of view of the sample plane via a detector device, wherein the field of view is detected with a frame rate up to about 2000 Hz and wherein the detected field of view has a size of about 150 μm to about 250 μm in a first dimension by about 100 μm to about 210 μm in a second dimension; and
(c) determining a change in the movement of the fluorescently labeled target protein in the presence of the compound,
   wherein a change in the movement of the target fluorescent protein in the presence of the compound relative to the motion of the target fluorescent protein in the absence of the compound identifies a biological interaction between the compound and the target fluorescent protein.

G. The present disclosure provides a method of identifying a biological interaction between a compound and a target fluorescent protein in a live cell comprising:
(a) contacting a sample comprising a population of live cells with the compound, wherein the live cells comprise target fluorescent proteins;
(b) tracking the movement of a plurality of individual target fluorescent proteins in a plurality of live cells in the sample, wherein said tracking comprises:
   (i) illuminating a field of view in a sample plane disposed within the sample with a light beam to cause fluorescence by a subset of the target fluorescent proteins in the live cells; and
   (ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension and wherein equal to or greater than 95% of the detected field of view achieves sufficient laser illumination for tracking protein movement; and
(c) determining a change in the movement of the fluorescently labeled target protein in the presence of the compound,
wherein a change in the movement of the target fluorescent protein in the presence of the compound relative to the motion of the target fluorescent protein in the absence of the compound identifies a biological interaction between the compound and the target fluorescent protein.

G1. The method of A-G, wherein the average change in movement of the target fluorescent proteins in the presence of the compound is at least 1%, at least 5%, at least 10% relative to the change observed in the absence of the compound.

G2. The method of any one of A-G1, wherein the change in movement is detected as an increase in immobile trajectories indicating an increase in bound (f_{bound}) target fluorescent protein.

G3. The method of any one of A-G1, wherein the change in movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(i) state occupation via inference.

G4. The method of any one of A-G3, wherein the subset of the target fluorescent proteins are in about 30 to about 80 live cells illuminated in the field of view of the sample plane.

G5. The method of any one of A-G4, wherein the target fluorescent protein interacts in a larger molecular assembly.

G6. The method of G5, wherein the target fluorescent protein is a ligand.

G7. The method of G5, wherein the target fluorescent protein is a receptor.

G8. The method of any one of A-G7, wherein the biological interaction is a direct interaction.

G9. The method of G8, wherein the direct interaction comprises binding of the compound to the target fluorescent protein.

G10. The method of any one of A-G7, where the biological interaction is an indirect interaction.

G11. The method of G10, wherein the indirect interaction comprises the compound agonizing or antagonizing a larger molecular assembly comprising the target fluorescent protein.

H. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise target fluorescent protein; and
   (iii) wherein the plurality of samples are contacted with distinct concentrations of the compound across a range of compound concentrations;
(b) tracking the movement of individual target fluorescent proteins in a plurality of live cells of a sample, wherein said tracking comprises:
   (i) illuminating a field of view in a sample plane disposed within the sample with a light beam to cause fluorescence by at least a subset of the target fluorescent proteins in the live cells; and
   (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and
(c) determining a change in the movement of a target fluorescent protein in the presence of the compound; and
(d) repeating steps (b)-(c) for each of the plurality of samples across the range of compound concentrations,
   wherein a change in the movement of the target fluorescent protein in the presence of the compound across the concentration range indicates the dose response of the compound.

I. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations;
(b) tracking the movement of individual target fluorescent proteins in a plurality of live cells of a sample, wherein said tracking comprises:
   (i) illuminating a field of view in a sample plane disposed within the sample with a light beam to cause fluorescence by at least a subset of the target fluorescent proteins in the live cells, wherein the subset of the target fluorescent proteins produces up to about 1,000,000 molecular trajectories in a single detected field of view; and
   (ii) detecting the fluorescence from one or more of the target fluorescent proteins in the detected field of view of the sample plane via a detector device, wherein the detected field of view has a size of about 150 μm to about 250 μm in a first dimension by about 100 μm to about 210 μm in a second dimension; and
(c) determining a change in the movement of the target fluorescent protein in the presence of the compound; and (d) repeating steps (b)-(c) for each of the plurality of samples across the range of compound concentrations,
wherein a change in the movement of the target fluorescent protein in the presence of the compound across the concentration range indicates the dose response of the compound.

J. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations;
(b) tracking the movement of individual target fluorescent proteins in a plurality of live cells of a sample, wherein said tracking comprises:
   (i) illuminating a field of view in a sample plane disposed within the sample with a light beam to cause fluorescence by at least a subset of the target fluorescent proteins in the live cells, wherein the field of view is illuminated using a 0.1 to 1 msec stroboscopic laser pulse;
   (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and
(c) determining a change in the movement of a target fluorescent protein in the presence of the compound; and
(d) repeating steps (b)-(c) for each of the plurality of samples across the range of compound concentrations,
wherein a change in the movement of the target fluorescent protein in the presence of the compound across the concentration range indicates the dose response of the compound.

K. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations;
(b) tracking the movement of individual target fluorescent proteins in a plurality of live cells of a sample, wherein said tracking comprises:
   (i) illuminating a field of view in a sample plane disposed within the sample with a light beam to cause fluorescence by at least a subset of the target fluorescent proteins in the cells;
   (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and
   (iii) detecting the fluorescence from a plurality of the target fluorescent proteins in the detected field of view of the sample plane at a rate of more than about 100,000 detected field of views per day; and
(c) determining a change in the movement of a target fluorescent protein in the presence of the compound; and
(d) repeating steps (b)-(c) for each of the plurality of samples across the range of compound concentrations,
wherein a change in the movement of the target fluorescent protein in the presence of the compound across the concentration range indicates the dose response of the compound.

L. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations;
(b) tracking the movement of individual target fluorescent proteins in a plurality of live cells of a sample, wherein said tracking comprises:
   (i) illuminating a field of view in a sample plane disposed within the sample with a light beam to cause fluorescence by at least a subset of the target fluorescent proteins in the live cells;
   (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and
   (iii) achieving, based on a single field of view, a z-factor of >0.5; and
(c) determining a change in the movement of a target fluorescent protein in the presence of the compound; and
(d) repeating steps (b)-(c) for each of the plurality of samples across the range of compound concentrations,
wherein a change in the movement of the target fluorescent protein in the presence of the compound across the concentration range indicates the dose response of the compound.

M. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations;
(b) tracking the movement of individual target fluorescent proteins in a plurality of live cells of a sample, wherein said tracking comprises:
   (i) illuminating a field of view in a sample plane disposed within the sample with a light beam to cause fluorescence by at least a subset of the target fluorescent proteins in the live cells; and
   (ii) detecting the fluorescence from one or more of the target fluorescent proteins in the sample plane via a detector device, wherein the field of view is detected with a frame rate up to about 2000 Hz and wherein the detected field of view has a size of about 150 μm to about 250 μm in a first dimension by about 100 μm to about 210 μm in a second dimension; and
(c) determining a change in the movement of a target fluorescent protein in the presence of the compound; and
(d) repeating steps (b)-(c) for each of the plurality of samples across the range of compound concentrations,
wherein a change in the movement of the target fluorescent protein in the presence of the compound across the concentration range indicates the dose response of the compound.

N. The present disclosure provides a method of determining a dose response of a compound that induces a change in the movement of a target fluorescent protein in a live cell comprising:
(a) contacting a plurality of samples with the compound,
   (i) wherein each sample comprises a population of live cells;
   (ii) wherein the live cells comprise the target fluorescent protein; and
   (iii) wherein the plurality of samples are contacted with distinct concentrations of the compound across a range of compound concentrations;
(b) tracking the movement of individual target fluorescent proteins in a plurality of live cells of a sample, wherein said tracking comprises:
   (i) illuminating a field of view in a sample plane disposed within the sample with a light beam to cause fluorescence by at least a subset of the target fluorescent proteins in the live cells; and
   (ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension and wherein equal to or greater than 95% of the detected field of view achieves sufficient laser illumination for tracking protein movement; and
(c) determining a change in the movement of a target fluorescent protein in the presence of the compound; and
   (d) repeating steps (b)-(c) for each of the plurality of samples across the range of compound concentrations,
wherein a change in the movement of the target fluorescent protein in the presence of the compound across the concentration range indicates the dose response of the compound

N1. The method of H-N, wherein the average change in movement of the target fluorescent proteins in the presence of the compound is at least 1%, at least 5%, at least 10% relative to the change observed in the absence of the compound.

N2. The method of any one of H-N1, wherein the change in movement detected is an increase in immobile trajectories indicating an increase in bound (f_{bound}) target fluorescent protein.

N3. The method of any one of H-N1, wherein the movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(i) state occupation via inference.

N4. The method of any one of H-N3, wherein the target fluorescent protein interacts in a larger molecular assembly.

N5. The method of N4, wherein the target fluorescent protein is a ligand.

N6. The method of N4, wherein the target fluorescent protein is a receptor.

N7. The method of any one of H-N6, wherein the biological interaction is a direct interaction.

N8. The method of N7, wherein the direct interaction comprises binding of the compound to the target fluorescent protein.

N9. The method of any one of H-N6, where the biological interaction is an indirect interaction.

N10. The method of N9, wherein the indirect interaction comprises the compound agonizing or antagonizing a larger molecular assembly comprising the target fluorescent protein.

N11. The method of any one of H-N11, wherein the subset of the target fluorescent proteins are in about 30 to about 80 live cells illuminated in the field of view of the sample plane.

O. The present disclosure provides a microscopy system configured to identify a biological interaction between a compound and a target fluorescent protein in a live cell comprising:
(a) a stage for supporting a sample, wherein the sample comprises a population of live cells, and wherein the live cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane and wherein the detected field of view has a size of about 150 μm to about 250 μm in a first dimension by about 100 μm to about 210 μm in a second dimension;
(d) a detector device for monitoring the light-based response from the target fluorescent proteins in the presence of the compound;
(e) a memory; and
(f) a processor in communication with the memory and the detector device, wherein the processor is capable of determining the change in the movement of the target fluorescent protein in the presence of the compound relative to the absence of the compound.

P. The present disclosure provides a microscopy system configured to identify a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane, wherein the subset of the target fluorescent proteins produces up to about 1,000,000 molecular trajectories in a single field of view and wherein the detected field of view has a size of about 150 μm to about 250 μm in a first dimension by about 100 μm to about 210 μm in a second dimension;
(d) a detector device for monitoring the light-based response from the target fluorescent proteins in the presence of the compound;
(e) a memory; and
(f) a processor in communication with the memory and the detector device, wherein the processor is capable of determining the change in the movement of the target fluorescent protein in the presence of the compound relative to the absence of the compound.

Q. The present disclosure provides a microscopy system configured to identify a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample, wherein a field of view is illuminated using a 0.1 to 1 msec stroboscopic laser pulse;
(c) an objective for focusing the light beam on the sample in the sample plane, wherein a subset of the target fluorescent proteins in the sample are disposed in a detected field of view of the sample plane and wherein the detected field of view has a size of about 150 μm to about 250 μm in a first dimension by about 100 μm to about 210 μm in a second dimension;
(d) a detector device for monitoring the light-based response from the target fluorescent proteins in the presence of the compound;
(e) a memory; and
(f) a processor in communication with the memory and the detector device, wherein the processor is capable of determining the change in the movement of the target fluorescent protein in the presence of the compound relative to the absence of the compound.

R. The present disclosure provides a microscopy system configured to identify a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a plurality of the target fluorescent proteins in the sample are disposed in a detected field of view of the sample plane and wherein the detected field of view has a size of about 150 μm to about 250 μm in a first dimension by about 100 μm to about 210 μm in a second dimension;
(d) a detector device for monitoring the light-based response from the target fluorescent proteins in the presence of the compound, wherein said monitoring comprises detecting the fluorescence from a plurality of the target fluorescent proteins in the detected field of view of the sample plane at a rate of more than about 100,000 detected field of views per day;
(e) a memory; and
(f) a processor in communication with the memory and the detector device, wherein the processor is capable of determining the change in the movement of the target fluorescent protein in the presence of the compound.

S. The present disclosure provides a microscopy system configured to identify a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a plurality of the target fluorescent proteins in the sample are disposed in a detected field of view of the sample plane and wherein the detected field of view has a size of about 150 μm to about 250 μm in a first dimension by about 100 μm to about 210 μm in a second dimension;
(d) a detector device for monitoring the light-based response from the target fluorescent proteins in the presence of the compound;
(e) a memory; and
(f) a processor in communication with the memory and the detector device, wherein the processor is capable of determining the change in the movement of the target fluorescent protein in the presence of the compound,
wherein the system has a z-factor of >0.5 based on a single detected field of view.

T. The present disclosure provides a microscopy system configured to identify a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a plurality of the target fluorescent proteins in the sample are disposed in a detected field of view of the sample plane and wherein the detected field of view has a size of about 150 μm to about 250 μm in a first dimension by about 100 μm to about 210 μm in a second dimension;
(d) a detector device for monitoring the light-based response from the target fluorescent proteins in the presence of the compound, wherein the detector device has a frame rate up to about 2000 Hz;
(e) a memory; and
(f) a processor in communication with the memory and the detector device, wherein the processor is capable of determining the change in the movement of the target fluorescent protein in the presence of the compound.

U. The present disclosure provides a microscopy system configured to identify a biological interaction between a compound and a target fluorescent protein in a live cell comprising:
(a) a stage for supporting a sample, wherein the sample comprises a population of cells, and where the cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a plurality of the target fluorescent proteins in the sample are disposed in a detected field of view of the sample plane, wherein the detected field of view has a size of about 150 μm to about 250 μm in a first dimension by about 100 μm to about 210 μm in a second dimension and wherein equal to or greater than 95% of the detected field of view achieves sufficient laser illumination for tracking protein movement;
(d) a detector device for monitoring the light-based response from the target fluorescent proteins in the presence of the compound;
(e) a memory; and
(f) a processor in communication with the memory and the detector device, wherein the processor is capable of determining the change in the movement of the target fluorescent protein in the presence of the compound.

U1. The system of O-U, wherein the average change in movement of the target fluorescent proteins in the presence of the compound is at least 1%, at least 5%, at least 10% relative to the change observed in the absence of the compound.

U2. The system of O-U1, wherein the change in movement detected is an increase in immobile trajectories indicating an increase in bound (f_{bound}) target fluorescent protein.

U3. The system of any one of O-U1, wherein the movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(i) state occupation via inference.

U4. The system of any one of O-U3, wherein the target fluorescent protein interacts in a larger molecular assembly.

U5. The system of U4, wherein the target fluorescent protein is a ligand.

U6. The system of U4, wherein the target fluorescent protein is a receptor.

U7. The system of any one of O-U6, wherein the biological interaction is a direct interaction.

U8. The system of U7, wherein the direct interaction comprises binding of the compound to the target fluorescent protein.

U9. The system of any one of O-U6, where the biological interaction is an indirect interaction.

U10. The system of U9, wherein the indirect interaction comprises the compound agonizing or antagonizing a larger molecular assembly comprising the target fluorescent protein.

U11. The system of any one of O-U10, wherein the subset of the target fluorescent proteins are in about 30 to about 80 live cells illuminated in the field of view of the sample plane.

### 6. EXAMPLES

The presently disclosed subject matter will be better understood by reference to the following examples, which are provided as exemplary of the presently disclosed subject matter, and not by way of limitation.

### Example 1: Optical Line Scanning System

### A. Introduction

Single molecule localization microscopy (SMLM) techniques, such as single-molecule tracking (SMT), enable in situ measurements in live and fixed cells from which data-rich metrics can be extracted. SMT has been successfully applied to address a variety of biological questions and model systems, aiming to unravel the spatiotemporal regulation of molecular mechanisms that govern protein function, downstream pathway effects, and cellular function in healthy or pathological conditions. While powerful, SMLM often suffers from low throughput, illumination inhomogeneity, and microscope and user-induced technical biases. Due to technical limitations of scaling SMLM techniques, a tradeoff between spatial resolution, temporal resolution and throughput must be made, restricting these technologies to a few research groups.

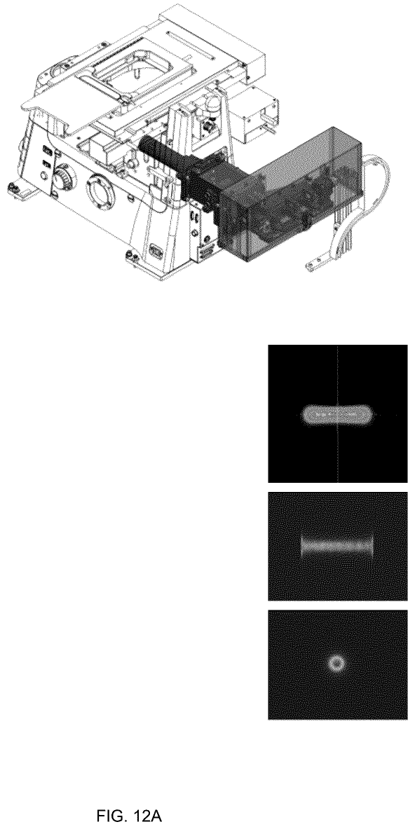

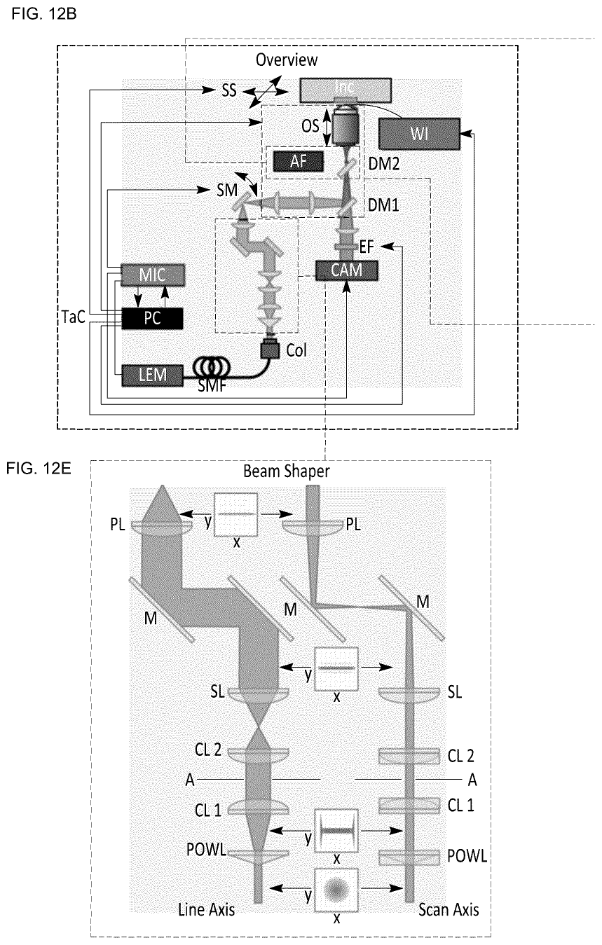

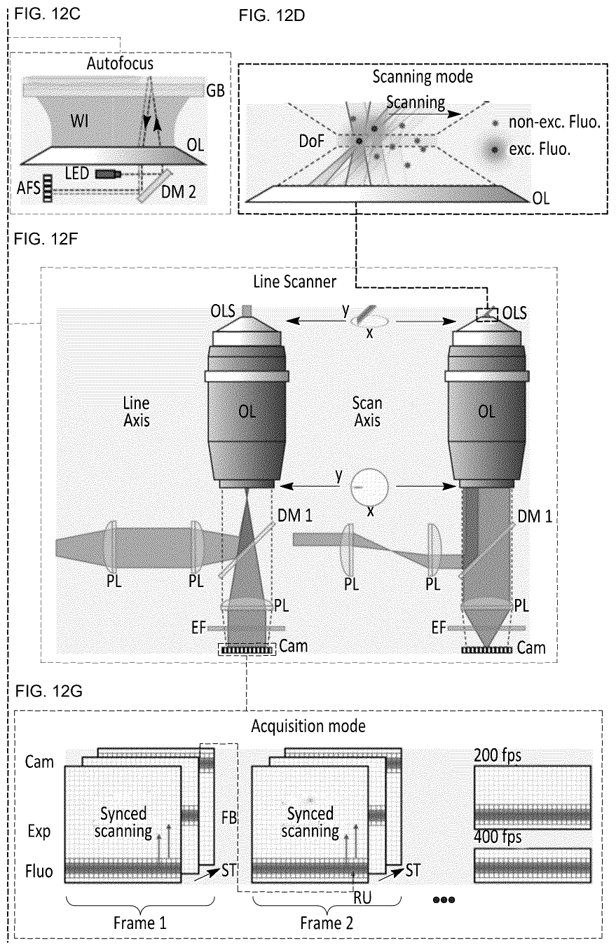

This example describes the development of an OLS system as disclosed herein to overcome the limitations of other SMLM techniques. Briefly, a thin optical light-sheet is shaped and focused into the back focal plane of the microscope's objective and scanned using a galvanometric mirror. This optical configuration results in a scannable oblique light sheet that can cover the full FOV of the water-immersion high NA objective (FIG. 11A and FIGS. 12A-12G). Further details regarding this exemplary OLS system are provided below.

### B. Exemplary OLS System

SMT Image acquisition for OLS datasets was performed on a custom-built microscope based on a Nikon Ti2, motorized stage, stage top environmental chamber (OKO labs), quadband filter cube (Chroma), custom laser launch with 405 nm, 561 nm, and 642 nm wavelengths, delivering >10 mW, >150 mW, and >150 mW of power to the back focal plane of the objective, respectively. The custom laser launch consists of three externally triggerable free-space laser sources (Cobolt 06-MLD; Huebner Photonics; 2RU-VFL-P-2000-560-M; MBP Communications Inc.; VFL-P-2000-642-M; MBP Communications Inc.).

The Oblique Line Scanning (OLS; FIGS. 12B and 12E) unit is attached to the back-port of the microscope providing optical excitation and scanning. The OLS unit receives collimated Gaussian-shaped optical excitation via a polarization-maintaining single-mode fiber coupled to a laser beam coupler. The laser excitation is sent through a combination of a Powell lens, custom-designed cylindrical lenses, and an achromatic lens to shape the beam into a laser line. The beam is guided over a set of two position-adjustable right-angle prisms followed by an aspherized achromatic lens to position the beam and focus the scan-axis onto galvanometric scanning mirrors, which is adjusted to position the beam at an offset of 3.8 mm to the central optical axis in the objective's back focal plane to achieve an illumination light sheet in the sample of at an inclination angle of 60 deg (FIG. 12F).

Fluorescence emission was passed through a high-speed filter wheel (Sutter Instruments) and collected with a backlit sCMOS camera (ORCA-Fusion BT, Hamamatsu). The sCMOS camera is operated in progressive mode with an exposure time of 407 μs at an internal line interval of 4.87 μs in order to achieve a virtual rolling slit of ˜200% of the optical excitation and fluorescence line width (FIG. 12G). Images were acquired with a 60×1.27 NA water immersion objective (Nikon). Environmental chamber was set to 37° Celsius, 95% humidity, and 5% CO₂.

System hardware control is realized in a custom-designed and user-configurable electric circuit board for software interfacing, synchronization, and device control. Data acquisition control is realized in a custom-designed and user-configurable acquisition script in MicroManager for raster scanning 384 well plates, a custom-designed automated focusing routine (FIG. 12C). 1 frame of Hoechst and Potomac Red channel were collected at the same frame rate for downstream registration of trajectories to nuclei and cytoplasms, respectively.

### C. Discussion

This example discloses OLS, a robust single-objective light-sheet based illumination and detection modality that achieves nanoscale spatial resolution and sub-millisecond temporal resolution across a 250×190 μm field of view to overcome the limitations of other SMLM techniques. OLS was developed with the purpose of enlarging the effective imaging area while homogenizing SNR across the camera chip to yield high quality SMLM and SMT raw image files with no compromise to the achieved spatiotemporal resolution. The relative simplicity of the optical configuration used in OLS renders this approach readily implementable on inverted microscopes equipped with either water- or oil-immersion high numerical aperture (NA) objectives and an sCMOS camera with light-sheet mode capability.

### Example 2: OLS High Throughput Single Molecule Tracking (htSMT)

### A. Introduction

This example describes exemplary industrial scale OLS htSMT techniques using the exemplary OLS system of Example 1 and the comparison of such an OLS system to a Highly Inclined and Laminated Optical Sheet (HILO) system. This example further describes systems incorporating such OLS htSMT techniques, hardware and software related to such OLS htSMT techniques, as well as methods of using such OLS htSMT techniques. For example, the OLS htSMT techniques described herein are capable of measuring protein movement in millions of cells per day. The OLS htSMT techniques described herein exhibit specific, robust, and reproducible results. The OLS htSMT techniques described herein can be used for a variety of applications including, but not limited to, classical drug discovery activities, such as compound library screening and the elucidation of SAR. Importantly, the OLS htSMT techniques described herein can be used to characterize both known and novel pathway contributions to interaction networks, such as protein signaling interaction networks.

### B. Results

a. Creation and Validation of an htSMT System

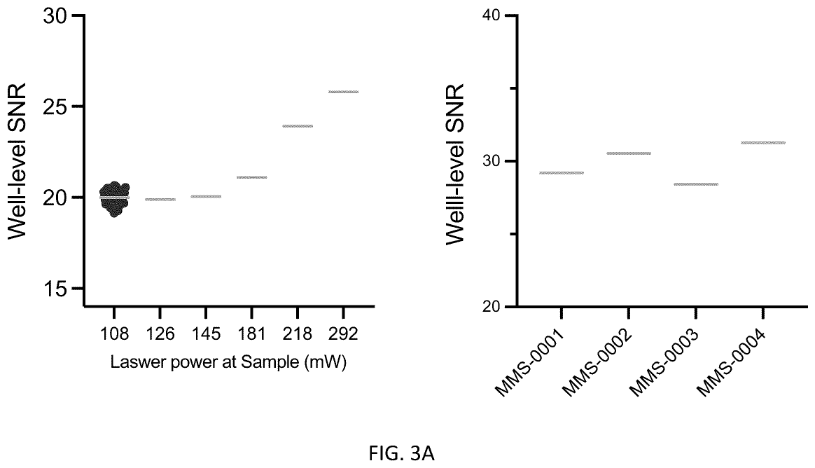

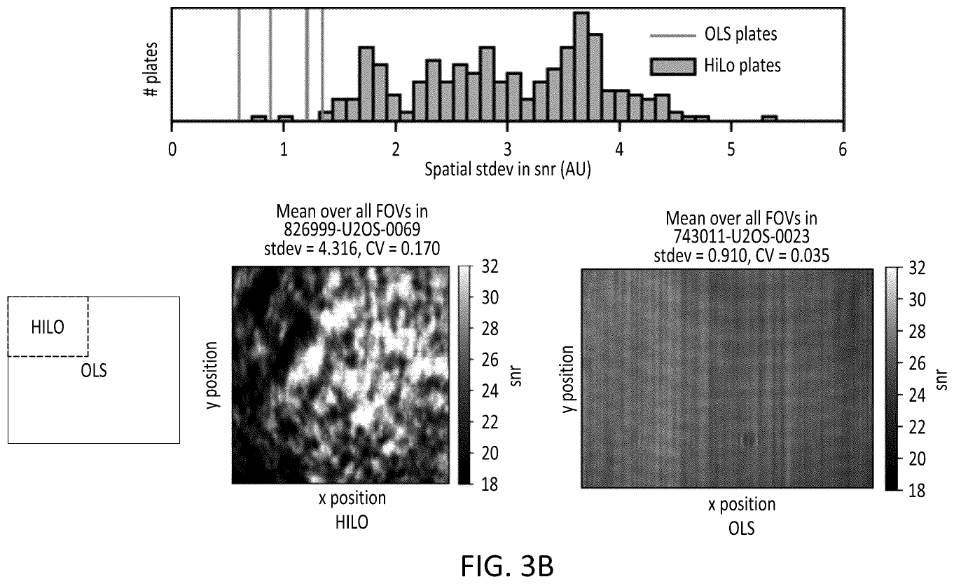

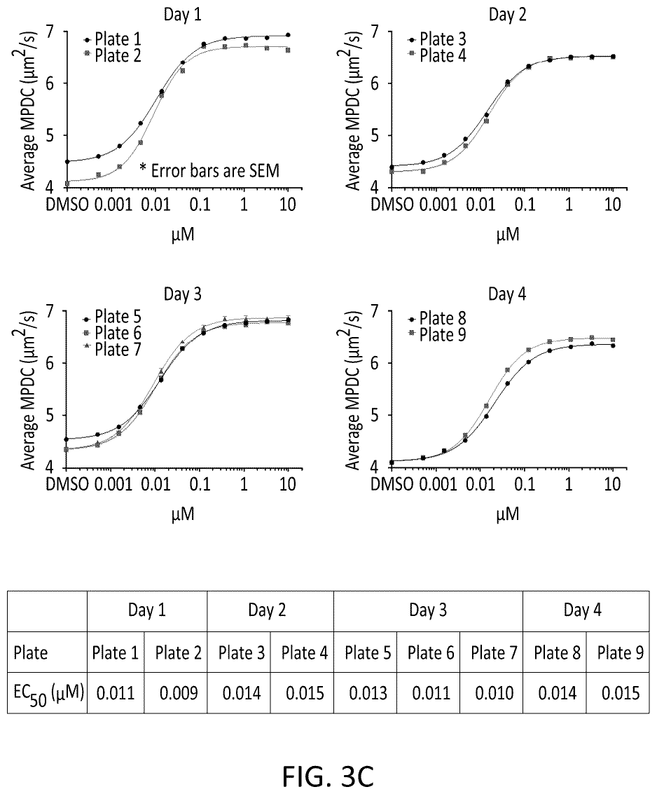

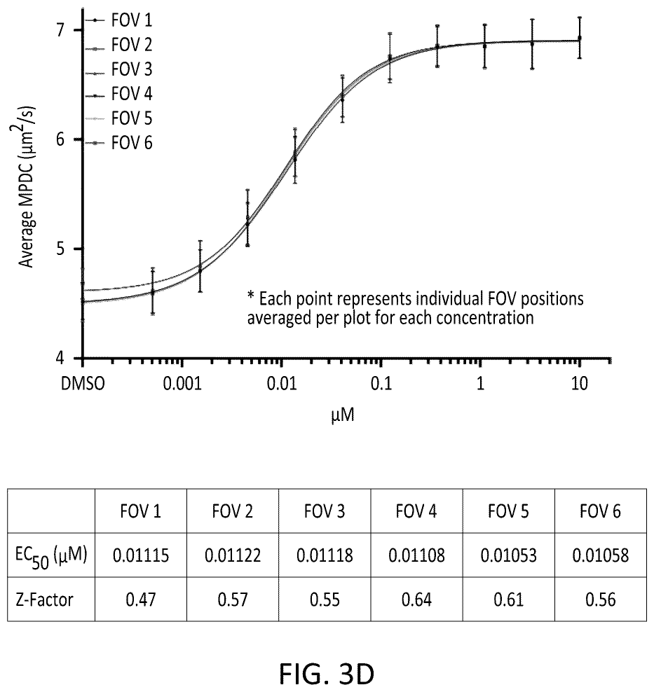

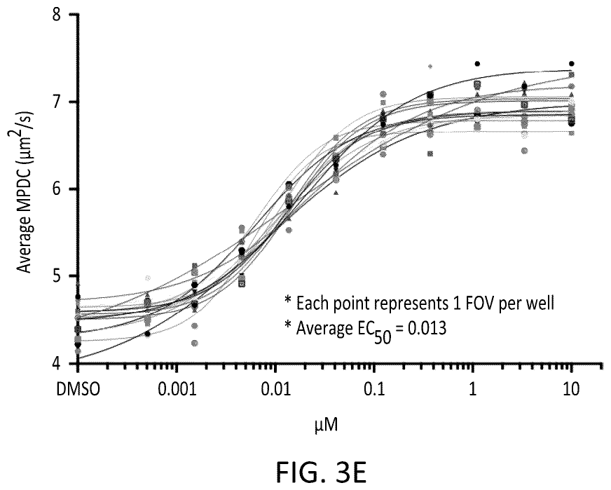

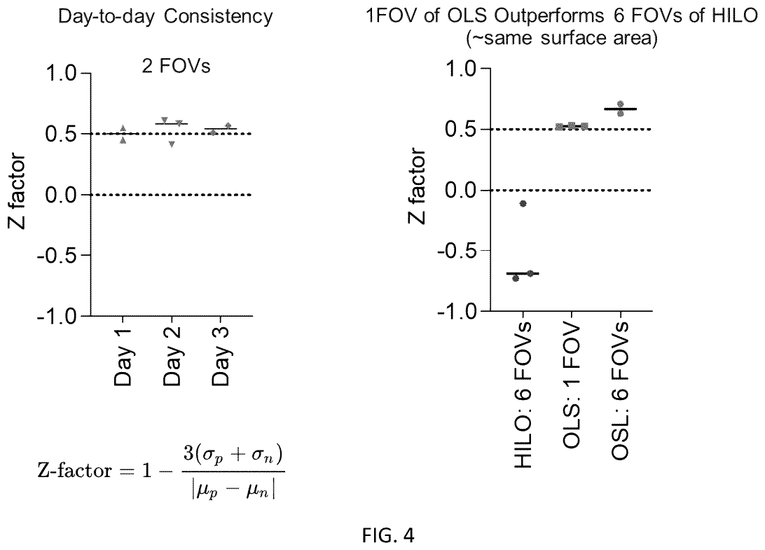

A robotic system capable of handling reagents, collecting high-quality, fast SMT image series, processing time-ordered raw images to yield molecular trajectories, and extracting features of biological interest within defined cellular compartments was developed (FIG. 1). To examine htSMT system performance various measures were performed indicating that the image acquisition systems and workflows of the present disclosure are amenable to robust htSMT analysis. For example, FIG. 3A depicts a laser titration experiment indicating the relationship of laser power at the sample (mW) to signal-to-noise ratio (SNR) (left panel), as well as the average SNR at the well-level across four image acquisition systems measuring six different 384 well plates per system (right panel). FIG. 3C depicts a dose-response experiment conducted on a halo-tagged protein with an established and well-characterized compound to assess plate to plate and day to day reproducibility (top panels) and the respective EC50s presented (bottom panel). FIG. 3D indicates that the systems described herein are configured to capture comparable protein diffusion coefficients per FOV per well, where each point represents individual FOV positions averaged per plot for each concentration (top panel) and both the EC50s and z-Factors are presented (bottom panel). FIG. 3E depicts the consistency of data across multiple wells and multiple experiments, where each point represents one FOV from 14 independently generated dose-response curves.

In addition to establishing the OLS workflows described herein are amenable to robust htSMT analysis, experiments were undertaken to compare the OLS-based workflows described herein with HILO-based approaches. For example, a comparison of Z-factors associated with the OLS-based data presented in FIG. 3D and FIG. 3E to data collected using a HILO-based approach clearly illustrates the improved performance of the OLS-based approach. These differences between OLS-based approaches and HILO-based approaches are particularly evident in FIG. 3B, which depicts differences heterogeneity in spatial SNR between the OLS systems of the instant disclosure and HILO-based approaches. The top panel compares the spatial standard deviations observed in OLS relative to a HILO-based approach. The bottom panel illustrates the difference in FOV between HILO and OLS-based approaches (left image), along with a comparison of the spatial heterogeneity across those FOVs for each of the HILO-based approach (middle image) and the OLS-based approach (right image).

Additional experiments were performed to illustrate the improved performance of the OLS-based approach compared to the HILO-based approach. For such comparisons, a U2OS cell line with the HaloTag genome-edited into the amino terminus of KEAP1 gene (Halo-KEAP1) was used. Initial imaging of Halo-KEAP1 sparsely labeled with the rhodamine dye Janelia Fluorophore 549 (JF₅₄₉) resulted in clear single molecule resolution from which analysis of spot detection, localization, tracking can be applied (FIG. 11B). The performance of the OLS system was benchmarked against a HILO implementation. 1.5 seconds of SMT data were collected in both HILO and OLS and the resulting trajectories were plotted (FIG. 11D). The average number of trajectories collected across the FOV increased from 25,765±4838 with HILO to 167,479±46,324 with OLS, matching the calculated 6-fold imaging field increase (FIG. 11E). SMT data was collected for 1,224 FOVs across a 384 well plate and the average signal to noise ratio (SNR) for all spots localized within each pixel of the FOV was calculated and a spatial SNR map was rendered (FIG. 11F). The standard deviation and average SNR per FOV were then summarized across 308 wells for each of OLS and HILO, demonstrating improved consistency and performance in SNR when comparing the two illumination modalities (FIG. 11G).

For HILO, samples were illuminated for 2 msec by pulsing the excitation laser for a subset of the camera exposure time. For OLS, given the scanning rate of the light-sheet, it was calculated that each fluorophore is only exposed to light for 400 usec. Given this shorter fluorophore integration time, more consistent point spread functions (PSFs) across different diffusion rates was expected. This hypothesis was tested by analyzing mean spot width of KEAP1 with and without KI-696 (FIG. 13A). With HILO illumination, there was a 4.4% increase in the mean 26 radius of single molecule PSFs which decreased to 1.4% for OLS (FIGS. 13B and 13D). While a 400 μsec strobe time would have provided a direct comparison in motion-induced blurring performance in OLS, it was found that within this integration time, HILO does not enable single molecule detection as the vast majority of PSFs do not pass the noise threshold (FIG. 13C).

One of the major advantage provided by OLS, is that during the scan of the inclined light sheet, out of focus illuminated emitters are outside of the recorded strip of pixels on the camera. To characterize this superior illumination-based optical sectioning method, samples consisting of increasing concentrations of His-HaloTag in solution were prepared to titrate protein labeling density and the downstream effect on SNR and PSF detection. This experiment strikingly captures the expected improvement in sectioning ability provided by OLS. A faster decrease in the number of detected localizations were observed in HILO, which was correlated to a decrease in SNR (FIGS. 13E and 13F). These results highlight that under OLS illumination, single PSFs were better detected irrespective of local PSF overlaps that can arise from increasing dye or protein concentration. Taken together with the reduction in motion blur, OLS provides the ability to track single particles at high density with high resolving performance.

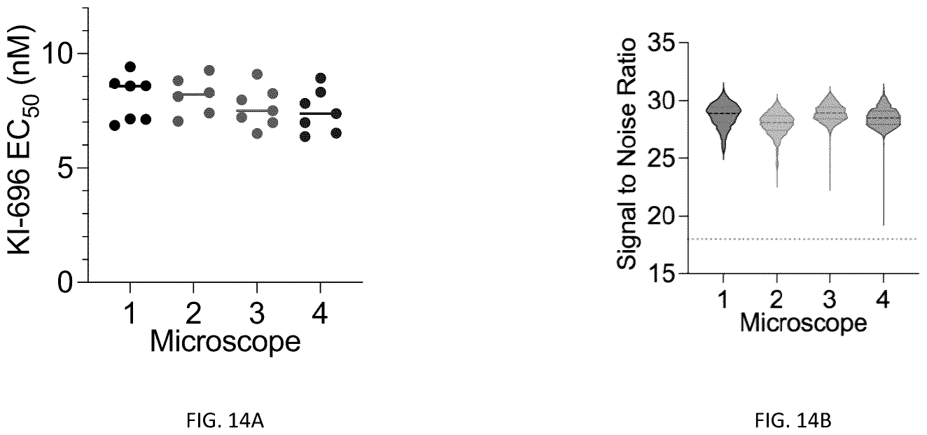

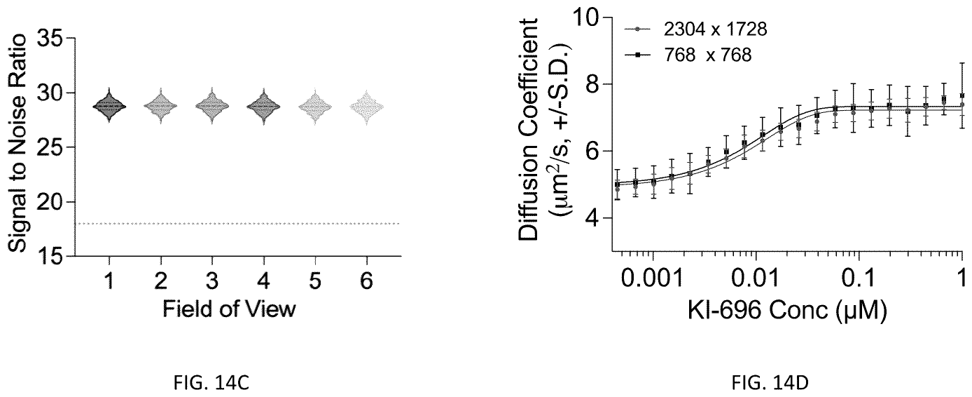

To further evaluate the reproducibility in illumination quality of the presently disclosed OLS optical systems, side-by-side SMT measurements on four distinct OLS-equipped microscopes using an automated system previously described. Six to seven 384 well plates were tested per microscope, treating Halo-KEAP1 with 20 concentrations of KI-696, a small molecule known to disrupts KEAP1 interaction with its binding partner NRF2, thereby increasing the fast-diffusing Halo-KEAP1 fraction, with 12 well replicates per concentration randomized across the plate and 6 FOVs per well. The average dose-response profiles per microscope were highly consistent, with a median increase in diffusion of 47-51%, and resulting median EC₅₀ values ranging between 7.37 and 8.58 nM across 4 independent microscopes (FIGS. 11C and 14A). The average FOV-level SNR per microscope was compared and all four microscopes provided a median SNR ranging between 28.08 and 28.89 (FIG. 14B). No change across subsequent FOVs captured within a single well was observed suggesting minimal disruption across the well upon imaging of a specific FOV (FIG. 14C). This implies that within this set of measurements, positional effects within a well do not appear to be present. Additionally, the effect of the large OLS FOV size on SMT sampling was directly characterized by comparing cropped regions of the same FOV to the large OLS sized-FOV. A significant increase in the variance was observed as the number of captured cells was shrunk to an area spanning 83×83 μm (FIG. 14D).

### C. Methods

a. Cell Lines

U2OS (ATCC Cat. No. HTB-96) can be grown in DMEM (Cat. No. 1056601, Gibco DMEM, high glucose, GlutaMAX Supplement, Thermofisher) supplemented with 10% Fetal Bovine Serum (Cat. No. 16000044, Thermofisher) and 1% pen-strep (Cat. No 15140122, Thermo Fisher) and maintained in a humidified 37° C. incubator at 5% CO₂ and subcultivated approximately every two to three days.

### Halo Tag-Expressing Cell Lines

For particular Target-HaloTag fusions, mammalian expression vectors containing the appropriate fusion gene under the control of a weak L30 promoter and containing a Neomycin resistance marker can be transfected into U2OS cells at 70% confluence using FuGENE 6 (Cat. No. E2691, Promega). Transfected cells can be selected with G418 (Cat. No. 10131027, Thermo Fisher) at 500 μg/mL, then clonally isolated. Clones expressing the desired fusion gene can be determined first by staining with 100 nM JF₅₄₉-HTL (Cat. No. GA1110, Promega) and 50 nM Hoechst 33342 and identifying clones with the expected distribution of JF₅₄₉ signal. A number of clones can be subsequently tested using SMT conditions for response to a control compound, and the most homogenous clones can subsequently be expanded for further testing.

To generate certain KEAP1-HaloTag cell lines (e.g., the cell line used in FIGS. 14A-14D), ribonucleoprotein (RNP) complexes included sgRNAs targeting either N- or C-terminal region (Integrated DNA Technologies-IDT) and Cas9 protein (PNA bio, Cat #CP01) were transfected together with linear dsDNA donors (IDT) using Lonza nucleofection method. Each donor consists of 200-300 bp homology arms specific for each target, codon optimized HaloTag sequence, and TEV linker (ENLYFQG) between the target and HaloTag. After transfection, the cells were incubated with Halo ligand JF₆₄₆ (Internal) and imaged with ImageXpress system (Molecular Device) to confirm HaloTag integration. Cells were then subjected to single cell sorting into 384-well plates. Clonal cells were expanded imaged with the ImageXpress system and genotyped by Sanger sequencing to confirm homogenous HaloTag integration.

b. Western Blot

Cells can be grown in the same conditions as described previously. 1.5×10⁶ cells can be seeded per well in a 6-well plate in DMEM overnight, followed by compound treatment (DMSO or 100 nM fulvestrant) the following day for 24 hours. Cells can then be lysed in 200 μL 1× Cell Lysis Buffer (catalogue number 9803, Cell Signaling). Protein lysate concentration can then be determined using BCA protein assay kit (Catalog number 23225, Pierce™ BCA Protein Assay Kit) following manufacturer instructions. Capillary Western Immunoassay can then be performed using Jess Protein Simple following manufacturer's instruction (protein simple, USA). Levels of anti-target antibody can be normalized to loading control β-tubulin (1:100, NC0244815 LI-COR 92642213, Thermo Fisher). The peaks can be analyzed with the Compass software (Protein Simple, USA).

c. OLS Single Molecule Tracking Sample Preparation

Cells can then be seeded on tissue culture-treated 384-well glass-bottom plates at 4500-6000 cells per well. Seeded cells can then be incubated at 37° C. and 5% CO₂ to allow adhesion overnight. For all SMT experiments, cells can be incubated with 5-100 pM of JF₅₄₉-HTL (Cat. No. GA1110, Promega) and 50 nM Hoechst 33342 for an hour in complete medium. Cells can then be washed three times in DPBS and twice in imaging media, which is fluoroBrite DMEM media (Cat. No. A1896701, Thermo Fisher) supplemented with GlutaMAX (Cat. No. 35050079, Thermo Fisher) and the same serum and antibiotics as growth media. Where appropriate, compounds can be serially diluted in an Echo Qualified 384-Well Low Dead Volume Source Microplate (0018544, Beckman Coulter) to generate dose-titration source material. Compounds can be administered at a final 1:1000 dilution in cell culture medium. Each dose of a compound can have at least 3 replicates per plate and up to 3 plate replicates are prepared sequentially, 20 DMSO control wells and 2 no dye control wells can be randomized across each plate. Compounds can be allowed to incubate for an hour at 37° C. prior to image acquisition.

d. Image Acquisition

Unless otherwise stated, all image acquisition using SMT was performed on a custom-built microscope, motorized stage, stage top environmental chamber, quad-band filter cube (Chroma), custom laser engine with 405 nm and 561 nm wavelengths to the back focal plane of the objective. Fluorescence emission was passed through a high-speed filter wheel (Finger Lakes Instruments) and collected with a backlit CMOS camera (Hammamatsu Orca Fusion run in light-sheet mode). Images were acquired with a 60×1.27 NA water immersion objective (Nikon). Environmental chamber was set to 37° Celsius, 95% humidity, and 5% CO₂. In certain implementations, each pixel is exposed for 400 microseconds and the full region of interest (ROI) takes 9 milliseconds total. The galvo position can then be reset in 1 millisecond, e.g., with the laser turned off, before recording another image. In such an implementation, 100 frames can be recorded per second. Additionally, or alternatively, a second setting can be employed that uses a smaller ROI in order to record 200 frames per second with the same 400 microsecond/pixel exposure and a 4 millisecond image recording time. Additionally, or alternatively, the galvo reset could be done faster.

e. Image Analysis

Image acquisition produced one JF₅₄₉ movie and one Hoechst per field of view. The JF₅₄₉ movie can be used to track the movement of individual JF₅₄₉ molecules, while the Hoechst movie can be used for nuclear segmentation. Tracking can be accomplished in three sequential steps—detection, subpixel localization, and linking-using a combination of existing methods. Briefly, spots can be detected using a generalized log likelihood ratio detector. After detection, the estimated position of each emitter can be refined to subpixel resolution using Levenberg-Marquardt fitting with an integrated 2D Gaussian spot model starting from an initial guess afforded by the radial symmetry method. Detected spots can be linked into trajectories using a custom modification of a hill-climbing algorithm. The same detection, subpixel localization, and linking settings can be used for all movies.

For nuclear segmentation, all frames of the Hoechst movie can be averaged to generate a mean projection. This mean projection can then be segmented with a neural network trained on human-labeled nuclei. Each spot can then be assigned to at most one nucleus using its subpixel coordinates.

To recover movement information from trajectories, state arrays can be used. For example, a Bayesian inference approach, with the “RBME” likelihood function and a grid of 100 diffusion coefficients from 0.01 to 100.0 μm²s⁻¹ and 31 localization error magnitudes from 0.02 to 0.08 μm can be used. After inference, localization error can be marginalized out to yield a one-dimensional distribution over the diffusion coefficient for each field of view. For single-cell analysis, SMT and nuclear segmentation can be performed, e.g., on a mixture of U2OS cells bearing H2B-HaloTag, HaloTag-CaaX, or free HaloTag. The marginal likelihood of each of a set of 100 diffusion coefficients on the set of trajectories within each segmented nucleus can then be evaluated. These marginal likelihood functions can be clustered with k-means, and the marginal likelihood functions for each cell can be ordered by their cluster index to produce the heat map. To estimate the fraction bound (f_{bound}), the state array posterior distribution below 0.1 μm²s⁻¹ can be integrated. To estimate the free diffusion coefficient (D_{free}), the mean of the posterior distribution above 0.1 μm²s⁻¹ can be computed.

f. Single Molecule Tracking Methods

Single molecule tracking (SMT) data were processed with a custom pipeline operating on image sequences produced by the microscope. Briefly, individual emitters were detected by applying a generalized log-likelihood ratio test to every 11×11 subwindow in the image as described above (Signal to noise ratio definition and quantification section below). Emitters were detected by identifying pixels with a log likelihood ratio exceeding 14. Detected emitters were localized to subpixel precision in a two-stage procedure. First, the subpixel location was estimated by computing points of maximum radial symmetry. Second, this estimate was used to seed an iterative Levenberg-Marquardt fitting routine to a 2D integrated Gaussian within a 11×11 pixel subwindow centered on the detection.

Localized emitters can be linked in time to produce trajectories using a modification of Sbalzerini's hill-climbing algorithm that uses Gibbs sampling to estimate data association uncertainty. In all SMT links longer than 1.25 μm were prohibited for cSMT and links over more than 2 gap frames to limit association error. Emitters were assigned to segmentation categories (nucleus, cytoplasm) by comparing their subpixel location with the semantic masks produced by the segmentation routine.

g. Data Analysis

Tracking results from the automated processing pipeline can be analyzed using KNIME or Spotfire (TIBCO). Individual f_{bound} or D_{free} measurements can be associated with experimental metadata and aggregated by condition. Change in f_{bound} can be calculated as the difference between the f_{bound} of each well and the median f_{bound} of DMSO in the same plate. Wells that had no cells in the field of view or in which the field of view was out of focus can be omitted from further analysis. Compounds can be assessed for assay interference using the median fluorescence intensity of the tracking channel and omitted if it they are more than 3 standard deviations higher than the median intensity of the DMSO wells. Similarly, plates where the active and negative controls could not be clearly resolved or where the significantly deviated from the performance of the rest of the screen can be removed from further analysis. Finally, compound with a variance more than three standard deviations higher than the average compound variance can be removed from downstream analysis. Z′-factor between the active controls on a plate and DMSO can be calculated. EC₅₀ values can be calculated in Prism (GraphPad) by first log-transforming the molecule concentrations and then fitting to a four-parameter logistic curve

h. Clustering Active Molecules

Chemical structure-based clustering can be performed on molecules identified as active. Molecular frameworks can be computed as known in the art and as implemented in Pipeline Pilot. Molecular frameworks can be clustered using functional class fingerprints (FCFP_4), e.g., with a similarity threshold cut-off of 0.3 Tanimoto distance.

i. Kinetic Experiments

Cells can be seeded into a 384-well plate the day before, dyed, and washed as described above. 1 well with a plurality of FOVs per well can be taken as a baseline reading. Then, while imaging, compound can be manually or robotically added to each well to a final concentration of 100 nM. Data can then be collected for the wells. A pause can be included between each FOV such that the entire imaging regime covers the assay window. Change in f_{bound} can be determined per-well relative to t=0.

For assays extending to 4 hours, the plate can be imaged twice with a plurality of FOVs per well with different FOV locations per readthrough to prevent photobleaching from impacting data.

j. Residence Time Imaging

Sample preparation and execution of residence time imaging experiments can be conducted in a similar manner to the single molecule tracking assay described above with a few exceptions. Samples can be dyed with 1-10 pM JF₅₄₉ (Promega) and 50 nM Hoechst 33342 for an hour. A plurality of frames per field of view can be collected with a camera integration time set to the desired time msec, and laser sources reduced to the desired mW at the objective. During image acquisition, lasers can be on continuously. Compound incubation can range from 1 to 4 hours.

k. Residence Time Analysis

Image processing, including spot detection, localization, and track reconnection can be performed using the same methods described above. Because residence time imaging selectively tracks slow-diffusing molecules, individual localizations can be limited in the distance of the maximum displacement for individual jump reconnections. Sets of trajectories for each field of view can be binned into 1-CDF distributions as previously described and fit to a two exponent decay model CDF(t)=A(Fe^{-k}^{_{fast}}^{t}+(1−F)e^{-k}^{_{slow}}^{t}).

1. Fluorescence Recovery after Photobleaching

Images can be acquired on a custom-built OLS microscope as described herein, e.g., in Example 1, with a Spectra Light Engine RS-232. Stimulation can be directed using a miniscanner coupled with a Coherent OBIS 561 nm 100 mW laser. All imaging can be performed using a 60×1.27 NA water immersion objective (Nikon). All experiments can be performed at 37° C. For FRAP experiments, cells can be seeded into a 384-well plate the day before, labeled with 50 nM HTL-JF₅₄₉, and washed as described above. Compound can be added to 100 nM final an hour before imaging. Then, a pre-bleach image can be acquired by averaging 10 consecutive images. Then 8-10 regions can then be bleached (2 background, 6-8 cells) and 2 regions in cells can be unbleached. Regions that are bleached are bleached at 10% power without scanning. For the next 30 seconds, an image can be acquired every 200 ms, then every 1 second for 2 minutes. The background-subtracted average intensity can be measured in the region of interest over time and normalized to the average of the fluorescence in the baseline images, then normalized to the unbleached regions to account for readout-induced photobleaching of fluorophores. Data from a plurality of cells can be pooled per experiment for three biological experiments.

m. HILO Microscopy

SMT image acquisition for HILO datasets was performed on a custom-built microscope based on a Nikon Ti2, motorized stage, stage top environmental chamber (OKO labs), quadband filter cube (Chroma), custom laser launch with 405 nm and 561 nm wavelengths, delivering >10 mW and >150 mW of power to the back focal plane of the objective, respectively. Fluorescence emission was passed through a high-speed filter wheel (Finger Lakes Instruments) and collected with a backlit sCMOS camera (ORCA-Fusion BT, Hamamatsu). Images were acquired with a 60×1.27 NA water immersion objective (Nikon). Environmental chamber was set to 37° C., 95% humidity, and 5% CO₂. For each field of view, 150 SMT frames were collected at a frame rate of 100 Hz, with a 2 ms stroboscopic laser pulse.

n. Measurements on Trajectories

When reporting the number of trajectories, singlets (trajectories with 1 detection) were excluded, as these do not contribute information to most dynamical estimates.

Average diffusion coefficients were computed with the mean squared displacement method (Dₑₛₜ=MSD_{2D}/4Δt). This estimator is expected to overestimate the diffusion coefficient by σ_{loc}²/Δt, where σ_{loc}² is the variance of the 1D localization error and Δt is the frame interval.

To resolve trajectories in multiple dynamical states, the coefficients of a Brownian mixture model over a grid of diffusion coefficient values and localization error values were inferred using state arrays, a variational Bayesian routine based on the Dirichlet process mixture. Mixture components were selected as the Cartesian product of 100 diffusion coefficients log-spaced between 0.01 and 100 μm²/s and 31 localization error values from 0.02 to 0.08 μm (1D standard deviations). Occupations are reported as the mean posterior probabilities of each diffusion coefficient marginalized over all values of localization error. To make inference tractable, inference was limited to 10000 trajectories randomly sampled from each well.

Bias estimation in single population samples was performed using analytical calculations that capture the probability of false linking and jump-length-distribution truncation due to a finite search radius.

o. Empirical Estimate of Linking Precision

To estimate the accuracy of the linking algorithm, a bootstrapping procedure was used. Detections from the first and second halves of the movie were superimposed, and the tracking algorithm was run on the resulting set of detections while blinded to the origin of each detection. From this, the fraction of links generated were computed where individual detections were joined from different halves of the movie. Since this fraction neither accounts for erroneous links between detections in the same half of the movie nor for the effects of photobleaching, it forms a lower bound on the linking error rate (ERLB).

p. Signal to Noise Ratio Definition and Quantification

Signal to noise ratio (SNR) is defined based on the likelihood ratio for a hypothesis test comparing: a target-absent condition, where the local image is modeled by the sum of a constant offset, and independent Gaussian-distributed noise; and a target-present condition, where the local image is modeled by the sum of a centrally located Gaussian peak (with known width but unknown amplitude), independent Gaussian-distributed noise, and a constant offset. The SNR is expressed as:

$$SNR = - \frac{{w}_{s}^{2}}{2} ln [ 1 - \frac{{( A ⊙ {h}_{G} )}^{2}}{( {A}^{2} ⊙ {h}_{u} ) - {( A ⊙ {h}_{u} )}^{2} / {w}_{s}^{2}} ]$$

where:
A is the image, cropped to the current region of interest (ROI);
wₛ is the side length, in pixels, of the square ROI;
\textcircled{*} is the inner product operator;
h_{G} is a zero-mean detection kernel, matched to the expected Gaussian target profile, and with

$$\sum {h}_{G}^{2} = 1$$

where the summation is taken over the ROI;
hᵤ is a uniform kernel (i.e., it has a value of 1 over the entire ROI).

### D. Discussion

Taken together, these results highlight the robustness and reproducibility of SMT measurements conducted using OLS illumination within a large FOV. These results further demonstrate the superior performance of OLS compared to HILO when characterizing the motion of fast-moving proteins at high labeling density. These data demonstrate how OLS, as a novel illumination scheme, enhances several properties of SMLM and SMT-based techniques. OLS provides a large FOV, finer sectioning ability and superior SNR, homogeneous illumination along with high spatiotemporal resolution relative to the established technique HILO. By reporting on results from OLS illumination modules implemented on four distinct microscopes, the robustness of OLS and the resulting consistency in result reproducibility was demonstrated. This robustness enables SMT measurements to be conducted agnostically on any microscope to test large compound libraries for drug screening. Additionally, the improvement in rejecting out of focus light enables better single molecule detection and localization, makes OLS suitable for conducting SMT on a variety of cellular systems and protein targets where previously, background fluorescence would be limiting. Consistent with this idea, high SNR SMT results in more complex cellular systems were achieved in spheroid cultures from immortalized cancer cells using OLS.

### Example 3: OLS Allows for Rapid SMT Data Capture Enabling Tracking of Fast-Moving Proteins

This example shows the impact higher frame rate acquisition has on assay window improvement and key imaging metrics of the OLS system of Example 1.

### A. Results

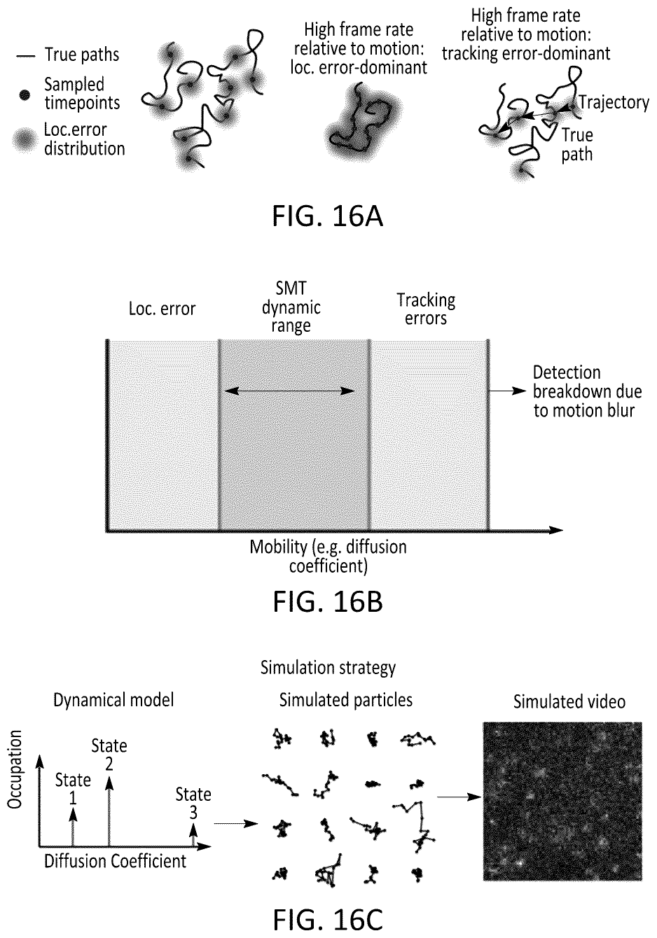

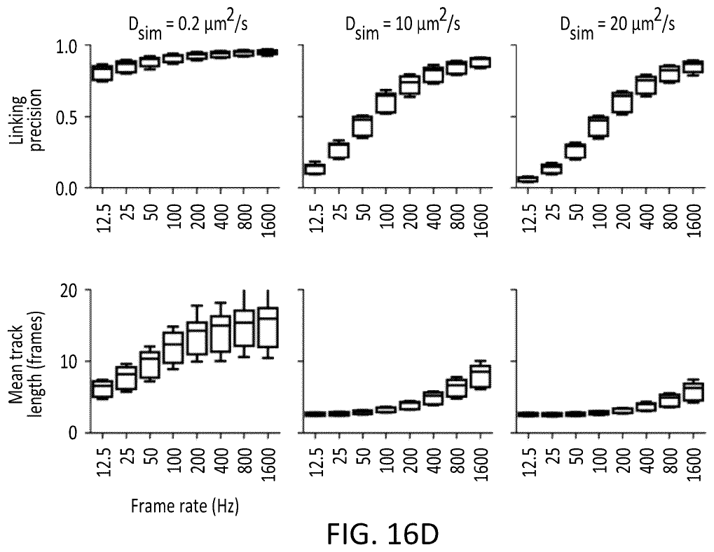

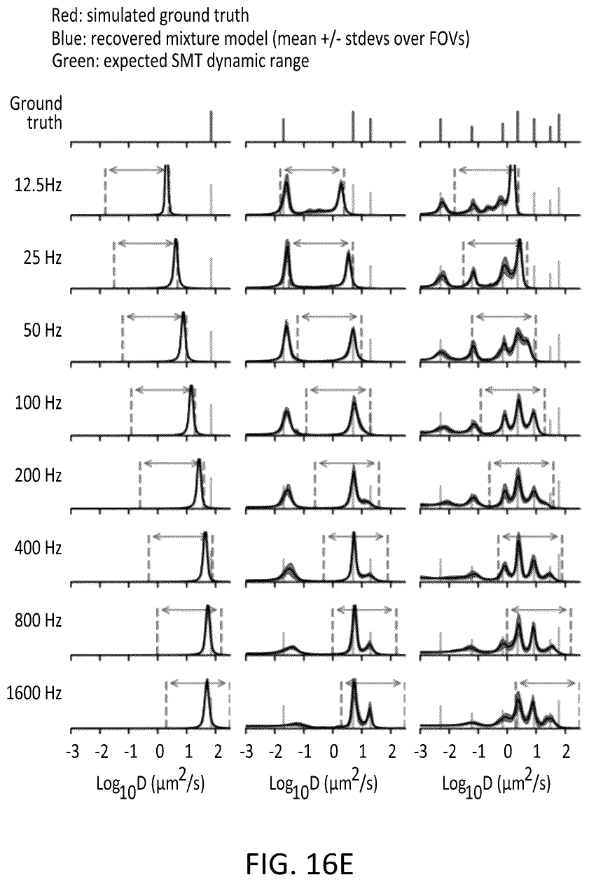

It was hypothesized that given the range and specificity of protein motion in live cells, there exists a set of optimal acquisition parameters for a given protein of interest. Frame rate correlates with other experimental factors such as localization error and tracking error to determine the information recoverable from SMT (FIGS. 16A and 16B). To understand these effects, optical-dynamical simulations were performed with complex mixtures of Brownian motions (FIG. 16C), then tracking was run on these simulated movies. Both mean track length and tracking fidelity improved with increasing frame rate, highlighting that the sampled FOV size is the only apparent compromise (FIG. 16D). The underlying dynamical model for each simulation was then estimated using state array analysis, a variational Bayesian method for recovering mixture models from observed trajectories. Increasing frame rate improved recovery of faster states but eventually degraded the recovery of slower ones (FIG. 16E). It was also noted that the lower and upper bound on the mean squared displacement (MSD) estimator of the diffusion coefficient, determined on one hand by localization error and on the other by the search radius used in tracking, roughly approximated this dynamic range (FIG. 16E, green dotted lines). These results suggested that tunable frame rates are a highly desirable property of an SMT imaging system.

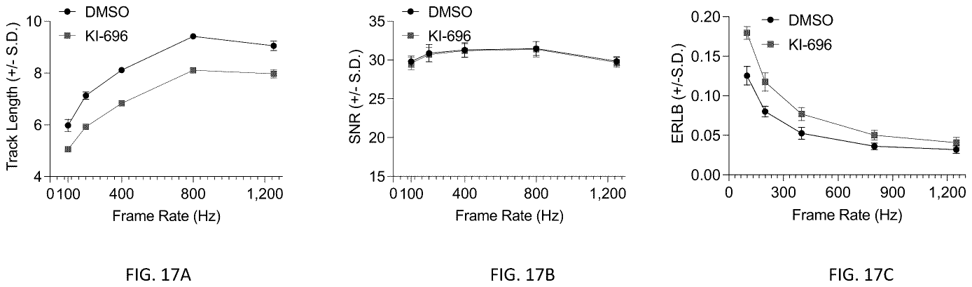

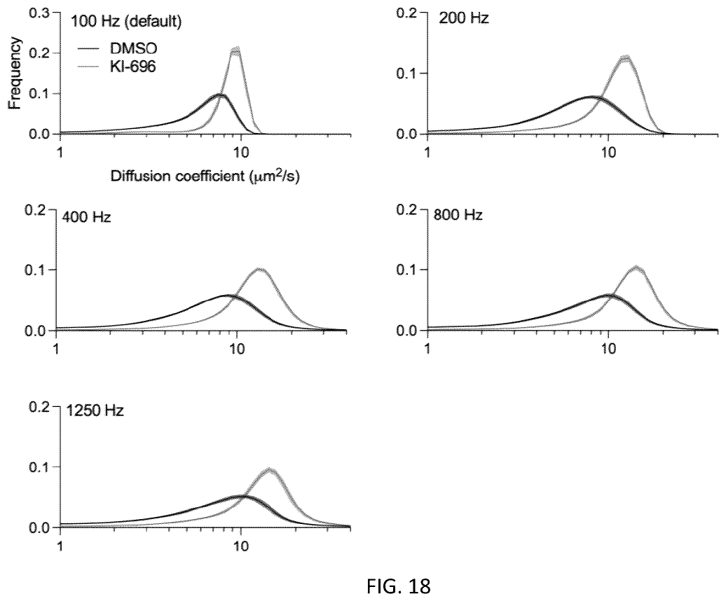

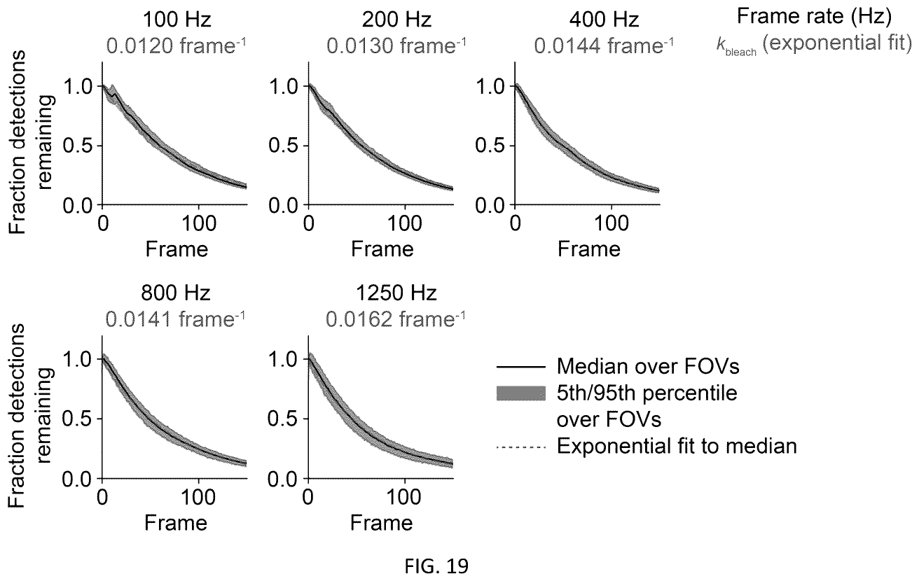

Turning to the experimental Halo-KEAP1 system described in Example 2, OLS-enabled SMT was ran with frame rates ranging from 100 to 1250 Hz (FIG. 15A). Similar to simulation results, both mean trajectory length and the estimated linking precision improved at higher frame rates (FIGS. 17A and 17C). Moreover, the OLS illuminator achieved this without degrading mean SNR (FIG. 17B) or bleaching rate per frame (FIG. 19). Running state array analysis, faster motion with increasing frame rate was recovered until the estimates stabilized at ˜9 μm²/s for DMSO-treated Halo-KEAP1 and ˜14 μm²/sec for KI-696-treated Halo-KEAP1 at 400 Hz (FIG. 18, FIG. 15B). Interestingly, it was noticed that 400 Hz may represent a point of diminishing return where the sampling frequency may be appropriate to capture the faster diffusing subset of KEAP1 under both DMSO and KI-696 treatment (FIG. 15B). Further, when the measured diffusion coefficients of Halo-KEAP1+/−KI-696 treatment at varying frame rates were simulated, simulations closely matched measured results (FIG. 15C). Together, these results demonstrated that the ability to increase frame rate utilizing the line scanning of OLS facilitates the accurate measure of fast protein diffusion in the cellular environment. While 400 Hz appears to be an appropriate sampling speed for KEAP1, it is anticipated that other important biochemical processes in live cells will be suitably captured only at significantly higher frame rates.

### B. Methods

a. Estimate of SMT Dynamic Range

To estimate the impact of frame rate on SMT dynamic range (as in FIG. 15C and FIG. 16), the bounds on the possible values of the mean squared displacement (MSD) estimator of the diffusion coefficient (\hat{D}) for a Brownian particle with Gaussian localization error were considered. The MSD estimator is biased upwards by localization error according to

$$\hat{D} = D + {σ}_{loc}^{2} / Δ t ,$$

where

$${σ}_{loc}^{2}$$

is the variance of the 1D localization error and Δt is the frame interval. Because D is nonnegative, this yields

$$\hat{D} \geq {σ}_{loc}^{2} / Δ t .$$

In the opposite limit, when the true jumps of the particle are much larger than the search radius R used for tracking, the jumps are uniformly distributed within the tracking range gate (a circle of radius R) and the MSD estimator of the diffusion coefficient is capped at R²/8Δt. Together, this yields the dynamic range estimate

$${σ}_{loc}^{2} / Δ t \leq \hat{D} \leq {R}^{2} / 8 Δ t .$$

Consequently, the effect of changing frame rate is to translate the dynamic range in the log \hat{D} domain. This simple model of dynamic range does not consider the impact of trajectory misconnection (which may change the upper bound) or the non-MSD estimate of diffusion coefficient in state arrays (which may reduce the lower bound).
b. Optical-Dynamical Simulations

To evaluate tracking methodology and the impact of frame rate on SMT dynamic range, optical-dynamical simulations were performed. These simulations use a scalar diffraction approximation to a paraxial imaging system with NA=1.2. Briefly, discrete mixtures of Brownian motions without state transitions at frame rates 12.5, 25, 50, 100, 200, 400, 800, or 1600 Hz in a cube with dimensions 45×45×8 μm (XYZ) were simulated. Particles were initialized at density 0.31 or 0.62 particles per cubic micron (depending on the simulation), and were subject to photobleaching at probability 0.03 per frame. Positions of particles coinciding with 500 μs pulses (modeling the stroboscopic illumination in HILO or the rolling shutter in OLS) were accumulated onto a simulated 2D camera via convolution with the system's 3D point spread function. This yielded a probability distribution of photon arrivals over all simulated camera pixels. Next, photon arrivals from this distribution as a Poisson process up to mean 90 or 125 photons per particle (depending on the simulation) were sampled. Finally, Gaussian read noise with RMS 3 photons was added, was multiplied by a 4.3 counts-per-photon gain factor, and the movie was discretized into 16-bit. This movie was then subjected to tracking and state array inference with settings identical to Halo-KEAP1 tracking.

### C. Discussion

OLS enables frame rates of at least 1250 Hz without compromising SNR and tracking fidelity. As illustrated with Halo-KEAP1, a frame rate of 400 Hz is required to fully characterize the increase in diffusion of drug-induced release from NRF2. It is anticipated that there are many biological processes that involve rapid protein motion that were previously unmeasurable with other SMT illumination methods. OLS provides an opportunity to tackle new protein and cellular mechanisms that are likely occurring at the sub-millisecond scale.

### Example 4: OLS Captures Intercellular Heterogeneity of Single Protein Dynamics

This example shows that the OLS system of Example 1 can be used to analyze intercellular heterogeneity in a sample.

### A. Results

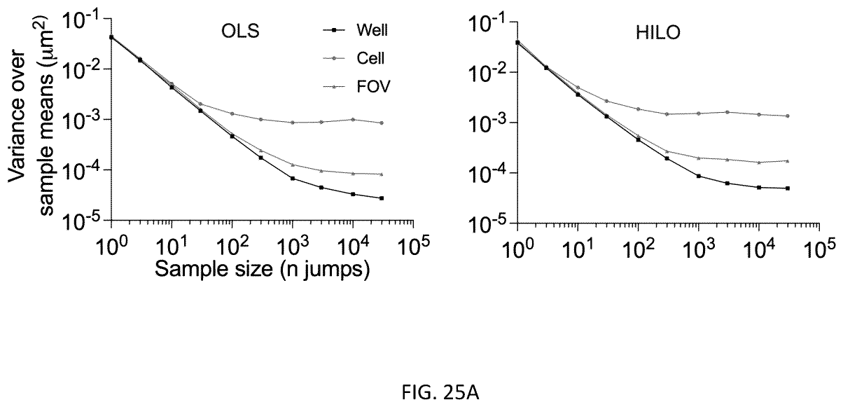

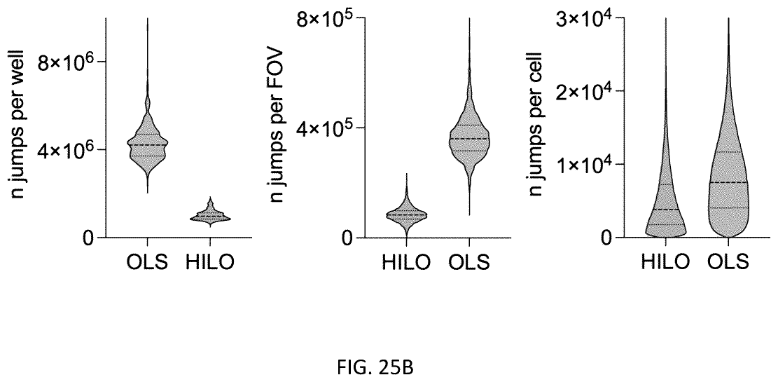

In assessing the consistency of SMT measurements it was determined that intercellular heterogeneity is the highest source of variance, exceeding FOV, well, plate or microscope-level variation. Cell-to-cell biases were at least an order of magnitude greater than FOV-to-FOV or well-to-well biases (FIGS. 25A-25B). This strongly indicates that biological heterogeneity is dominant over technical variation of OLS-based SMT measurements (FIG. 20A). Examples of cell heterogeneity include cell cycle, state of cell stress, and genetic variation. Single cell measurements such as large-FOV SMT can enable more nuanced measurements to better understand such heterogeneity. This example exemplifies such single-cell analysis by measuring the effect of cell cycle on proliferating cell nuclear antigen (PCNA) protein dynamics. PCNA is involved in DNA replication and hence relocalizes to replication foci during S-phase therefore exhibiting distinct and specific protein dynamics across the cell cycle.

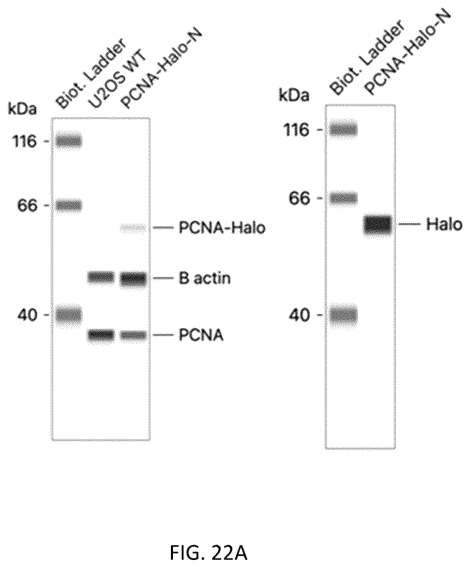

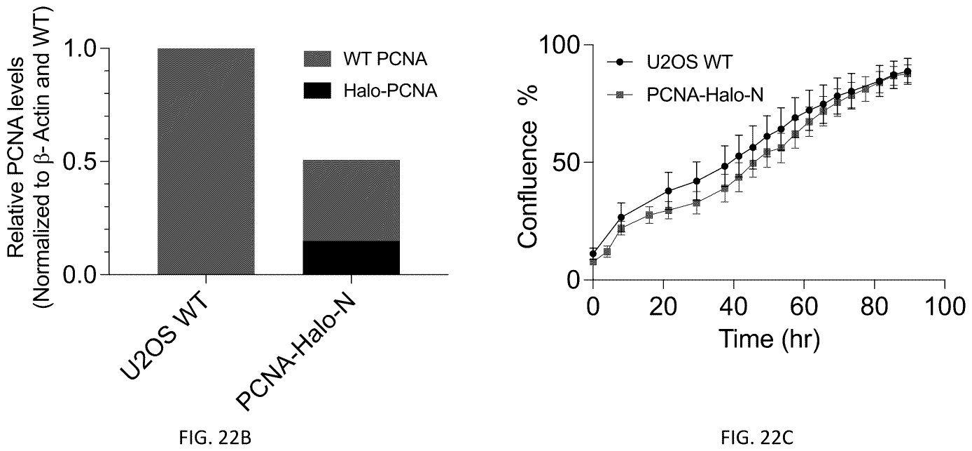

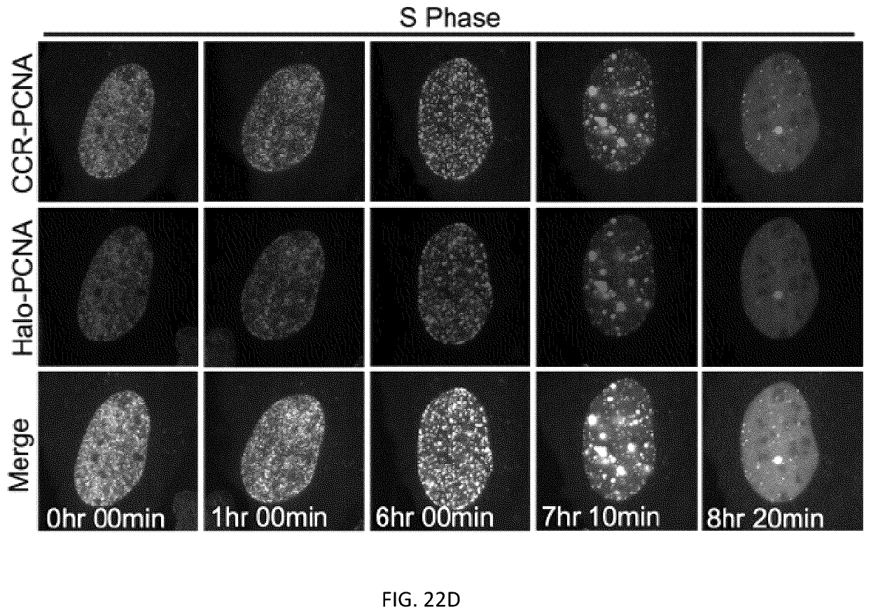

Halo-PCNA was introduced into U2OS isolated clones expressing sub-endogenous levels of tagged protein (FIGS. 22A and 22B) and found growth rates were not measurably affected by the expression of Halo-PCNA (FIG. 22C). Proper localization of Halo-PCNA was confirmed with colocalization analysis with an RFP-labeled anti-PCNA nanobody (FIG. 22D).

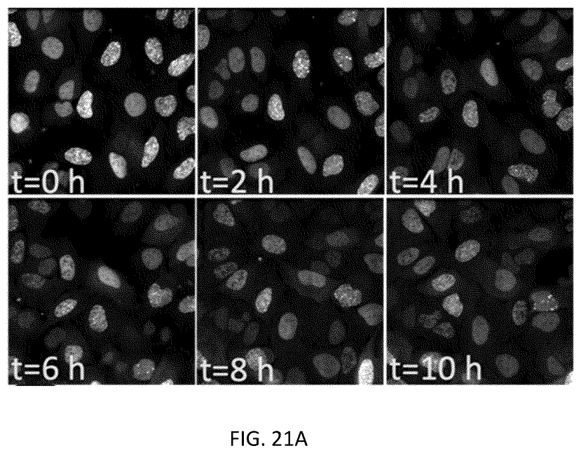

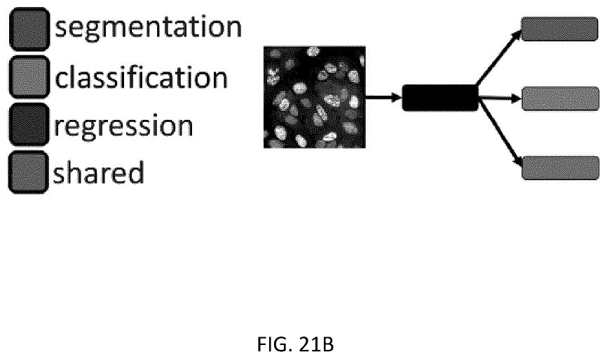

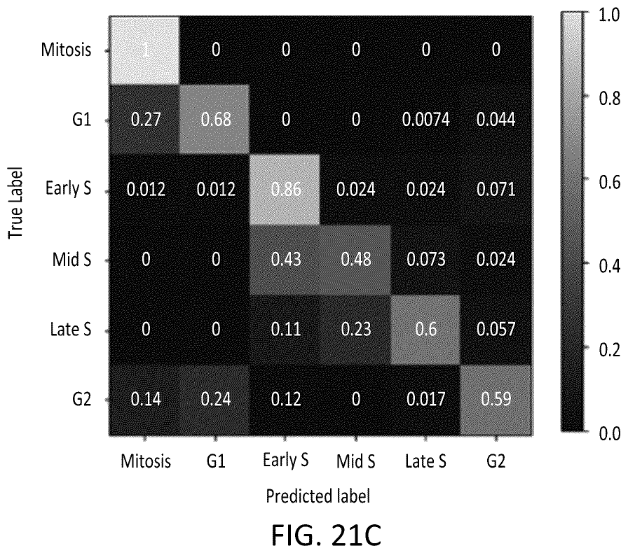

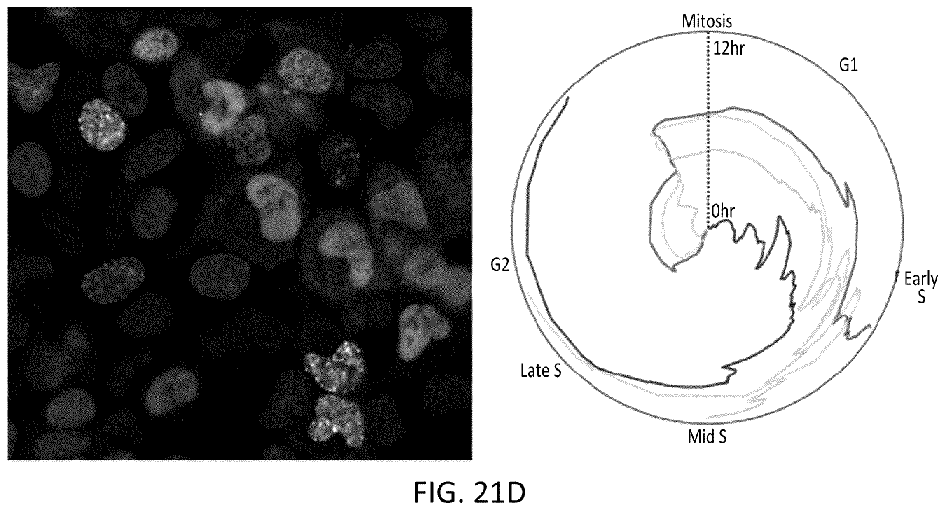

Time-lapse microscopy was conducted on Halo-PCNA labeled with 50 nM JFX₆₅₀, to achieve near-saturating labeling, for 12 hours at 12 frames per hour (FIG. 21A). A machine learning model was then trained using both manually assigned cell stages and time-based progression resulting in both a G1, early S, mid S, late S, G2 and mitosis classifier as well as a regression prediction across the continuum of the cell cycle (FIG. 21B). The predicted cell cycle classification performed well as compared to manual annotation (FIG. 21C). Clear progression of individual cells through the cell cycle was observed when regression prediction was plotted across the time series (FIG. 21D). To further validate the cell cycle phase assignment model, cell cycle progression at S-phases was blocked with thymidine or the G2-M transition was blocked with the CDK-1 inhibitor RO-3306. Both treatments lead to expected enrichment in the fraction of cells assigned to the respective cell phases (FIGS. 20B and 20C). Cells were then labeled with 10 μM JF₅₄₉ and 50 nM JFX₆₅₀ to enable simultaneous cell cycle assignment based on near-saturating labeling along with SMT measurements. Two peaks of mobility were observed at 0.043 and 9.88 μm²/s, likely representing PCNA associated at sites of DNA replication and free PCNA, respectively (FIG. 20D). When cells in each cell phase were analyzed separately, it became apparent that the slow-moving population of PCNA was exclusively found in cell predicted to be in S-phase and that there was a marked decrease in the fast-moving population (FIG. 20E). Mean diffusion coefficient of PCNA was then measured for 4,081 individual cells to characterize heterogeneity across the cell population, which is described in large part by cell cycle (FIG. 20F). Taken together these data provide an example of how large FOV SMT enabled by OLS allows for the capture and elucidation of cell-level heterogeneity of protein dynamics.

### B. Methods

c. Cell Line Engineering of CRISPR Knock-In Halo Tagged Proteins

All U2OS cells were cultured in Dulbecco's modified Eagle's medium (DMEM) (gibco) supplemented with 10% fetal bovine serum (Corning), 100 units/mL penicillin, 100 μg/mL streptomycin (Gibco), at 37° C. and 5% CO₂.

To generate Halotag-PCNA cell lines, ribonucleoprotein (RNP) complexes included sgRNAs targeting either N- or C-terminal region (Integrated DNA Technologies-IDT) and Cas9 protein (PNA bio, Cat #CP01) were transfected together with linear dsDNA donors (IDT) using Lonza nucleofection method. Each donor consists of 200-300 bp homology arms specific for each target, a codon optimized Halotag sequence, and TEV linker (ENLYFQG) between the target and Halotag.

After transfection, the cells were incubated with Halo ligand JF₆₄₆ (Internal) and imaged with ImageXpress system (Molecular Device) to confirm HaloTag integration. Cells were then subjected to single cell sorting into 384-well plates. Clonal cells were expanded imaged with the ImageXpress system and genotyped by Sanger sequencing to confirm homogenous HaloTag integration.

b. Cell Proliferation

6 well-plate cell proliferation-Cells were seeded into 6 well-plates (Fisher Scientific, 07-200-83) 150,000 cells/well and left at ambient temperature for 20 minutes to ensure homogeneous settling. The plate was imaged with the Incucyte (Sartorius) taking 9 images per well every 4 hours and phase masking algorithms were applied to determine cell confluence using the Incucyte software v.S3 2019A.

c. Western Blot Analysis

Protein was extracted from 1-2 million cells by lysing with 1× Cell Lysis Buffer (CST, #9803) diluted in UltraPure Sterile Water (Intermountain Life Sciences, 20804225) with 1× Halt Protease and Phosphatase Inhibitor Cocktail (Thermo Fisher Scientific, 78440). Samples were collected using cell scrapers (FisherScientific, 08-100-241) and placed on ice for 15 minutes and then centrifuged at 15,000 RPM for 10 minutes. The supernatant was transferred to a new tube. Protein concentration was determined with the Pierce BCA Protein Assay Kit (Thermo Fisher Scientific, A55864) according to the manufacturer's protocol. Samples were run on the automated western blot system Jess (ProteinSimple, 004-650) according to the manufacturer's protocols. The 12-230 kDa separation module was used for PCNA (CST, D3H8P, Rabbit, 1:100 dilution, #13110), β-Actin (CST, D6A8, Rabbit, 1:50 dilution, #8457S) and Halo (Promega, Mouse, 1:10 dilution, #G9211) detection. Samples were diluted to 0.3 mg/mL with 0.1× Lysis Buffer and 5× Master Mix and heat denatured at 95° C. for 5 minutes and all primary antibodies were diluted with Antibody Diluent 2. All reagents were loaded to a microplate and a 13 capillary cartridge was loaded onto the Jess western blot instrument. Run parameters were set using the COMPASS software v6.0.0 (ProteinSimple). Each protein's corresponding band was visualized using the COMPASS software and bands were normalized to the loading control b-Actin.

d. Machine Learning Architecture

Cell cycle as provided above can be automatically determined using an image (e.g., an image of JF₅₄₉-labeled PCNA). A machine learning model such as a neural network based on a U-Net architecture can be trained so that each pixel is assigned a label (FIGS. 21A-21D). The decoder of the architecture can comprise a set of three independent decoders D={D₁, D₂, D₃}. While the encoder E_{θ} that consumes the image is shared across D, each decoder can be configured to have a distinct set of parameters and be respectively trained to perform three different tasks: 1) nuclear segmentation, 2) cell cycle classification and 3) cell cycle regression. The network can be trained end-to-end using the average of three different losses that target these three tasks.

Each loss can be based on a class balanced cross entropy defined as:

$${CB}_{softmax} ( z , y ) = - \frac{1 - β}{1 - {β}^{{n}_{y}}} log ( \frac{exp ( {z}_{y} )}{{\sum }_{j = 1}^{c} exp ( {z}_{j} )} )$$

### Where:

Z is the predicted output of the model for all classes
C is the total number of classes
b is a hyperparameter between 0 and 1
n_{y} is the number of training samples per class y

The first loss can optimize the nuclear segmentation task and for an input, x, that represents a 2D image of fluorescently labeled PCNA be defined as:

$${L}_{segmentation} = {CB}_{softmax} ( {D}_{1} ( {E}_{θ} ( x ) ) , {y}_{1} )$$

where D₁(E_{θ}(x)) produces pixels level predictions for the three classes nucleus, nuclear edge, and background.

The second loss can optimize the cell cycle classification task and be defined as:

$${L}_{classification} = {CB}_{softmax} ( {D}_{2} ( {E}_{θ} ( x ) ) , {y}_{2} )$$

where D₂(E_{θ}(x)) produces pixels level predictions for the seven classes background, mitosis, G1, Early S, Mid S, Late S, and G2.

In order to produce a continuous representation of cell cycle categories (M, G1, Early S, Mid S, Late S, and G2), a target encoding method can be used so that the categories can be linearly arranged from 0-1 such that cells categorized as M are assigned to 0 and cells categorized as G2 are assigned to 0.8. In order to minimize complexities of aggregating the three losses, a cross entropy can be similarly used to train the regression task. The regression decoder D₃ can produce two outputs and the training data can be represented using a two valued vector (λ, 1−λ) where λ represents the 0-1 cell cycle value. The third loss can optimize the cell cycle regression task and be defined as:

$${L}_{regression} = {CB}_{softmax} ( {D}_{3} ( {E}_{θ} ( x ) ) , {y}_{3} )$$

Finally, the total loss for the network is the mean across these three losses:

$$L = \frac{{L}_{segementation} + {L}_{classification} + {L}_{regression}}{3}$$

Once the model has been trained, nuclear segmentation can be performed and either a categorical or continuous representation of cell cycle can be assigned to each nucleus based on the average pixel value that define the respective nucleus.

e. Assessment of Highest Source of Variance from Experiment: Jump Resampling Experiment

To evaluate the contribution of well-to-well, FOV-to-FOV, and cell-to-cell biases on estimated mean 2D jump length, a full plate (308 wells) of HILO and OLS data were acquired with DMSO-treated KEAP1-HaloTag labeled with JF₅₄₉ at 100 Hz. After excluding the outer ring of wells in the 384-well plate, this yielded a dataset with 308 wells, 12 FOVs per well, and a mean of ˜44 cells per FOV (for OLS) or ˜15 cells per FOV (for HILO). simple model of jump length was considered. Letting Y be the observed 2D jump length, Y was modeled as the sum

$$Y = Bwell + BFOV + Bcell + X$$

B_{well}, B_{FOV}, and B_{cell} are random variables modeling bias at the well, FOV, or cell levels, and X models the intrinsic stochasticity in jump length conditional on a particular well, FOV, and cell. The simplification is that B_{well}, B_{FOV}, B_{cell} and X are assumed to be independent. Under this simplification, Var(Y)=Var(B_{well})+Var(B_{FOV})+Var(B_{cell})+Var(X). A more physically realistic model would take into account the potential dependencies between these random variables.

To estimate Var(B_{well}), Var(B_{FOV}), Var(B_{cell}), and Var(X), the variance in sample means were computed for four different jump resampling schemes:
Sample N jumps from the whole plate. The resulting sample mean averages over all sources of variability (B_{well}, B_{FOV}, B_{cell} and X).
Sample a well, then sample N jumps from that well. The resulting sample mean averages over B_{FOV}, B_{cell}, and X, and the variance over these sample means is expected to approach Var(B_{well}) as N becomes large.
Sample a well, sample an FOV from that well, then sample N jumps from that FOV. The resulting sample mean averages over B_{cell} and X, and the variance over these sample means is expected to approach Var(B_{well})+Var(B_{FOV}) as N becomes large.
Sample a well, sample an FOV from that well, sample a cell from that FOV, then sample N jumps from that cell. The resulting sample mean averages over X only. The variance over these sample means is expected to approach Var(B_{well})+Var(B_{FOV})+Var(B_{cell}) as N becomes large.
   1000 rounds of sampling were performed for each sampling scheme and the variances of the biases B_{well}, B_{FOV}, and B_{cell} were estimated by the difference between the variances over sample means produced by each resampling scheme. As expected, only Var(X) depended on the sample size N and the other sources of variability were stable with respect to the sample size after ˜100 jumps (FIGS. 25A-25B).

### Discussion

This analysis suggests that the greatest source of variation in SMT measurement stems from cell heterogeneity in protein motion. The large FOV enabled by OLS allows for the simultaneous capture of upwards of 50 U2OS cells in culture, enabling the capture of inter-cell heterogeneity. This was exemplified with PCNA where it was possible to simultaneously assign the cell cycle phase along with monitoring protein dynamics. These results clearly show that PCNA protein dynamics are slower during S-phase which correlates to a protein enrichment at sites of DNA replication. During G1, G2 and M phases, a drastic increase in dynamics corresponding to the majority of PCNA being homogeneously distributed throughout the nucleus was observed. These findings are consistent with previous characterization of PCNA dynamics but there are important improvements in the approach presented here. Cell cycle was predicted computationally through machine learning models using localization of PCNA as opposed to manual assignment. Additionally, the OLS platform enabled rapid and automated capture and analysis of thousands of cells versus tens of cells per condition typically analyzed using manual SMT approaches. When characterizing protein motion in heterogeneous cell population and potential rare cell sub-types, automated cell classification along with scaled SMT data collection and analysis will be critical to enable work that is comparable with flow cytometry and other single-cell analysis techniques.

### Example 5: OLS is Amenable to a Variety of SMLM Techniques and Acquisition Schemes

This example provides an extension of applications using the OLS system of Example 1 that leverage the strength of OLS through homogeneous illumination, robustness, high spatiotemporal resolution and imaging speed.

### A. Results

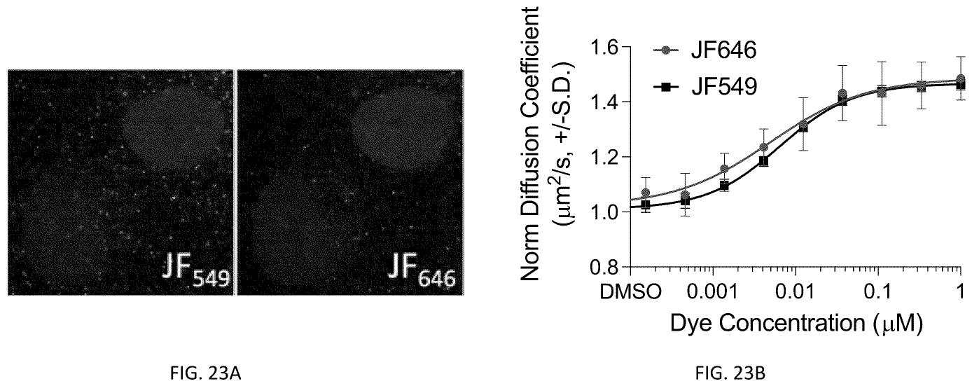

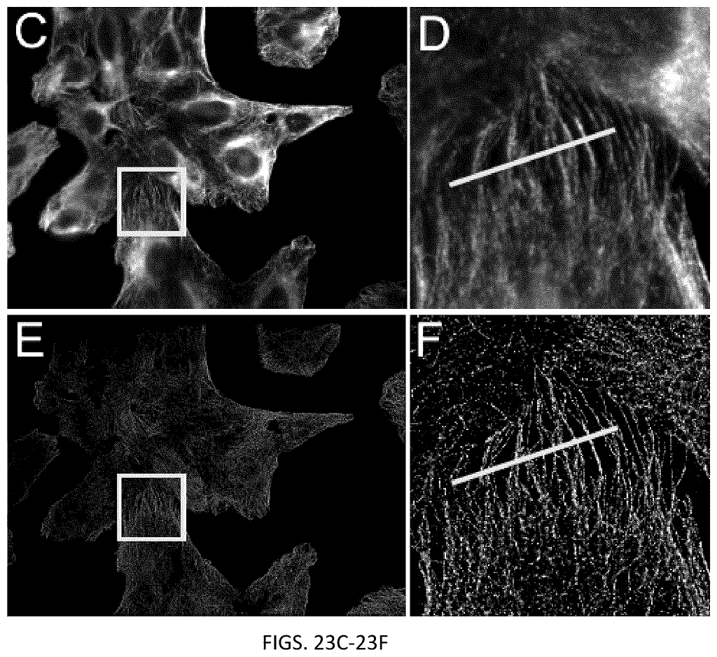

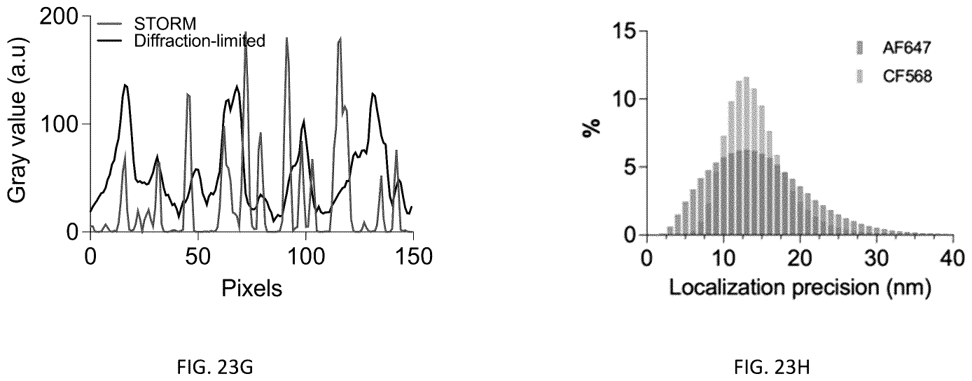

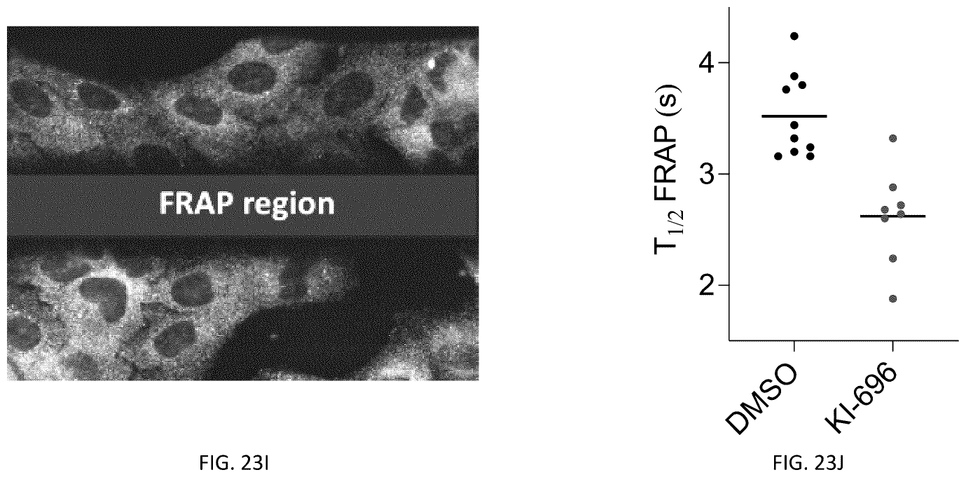

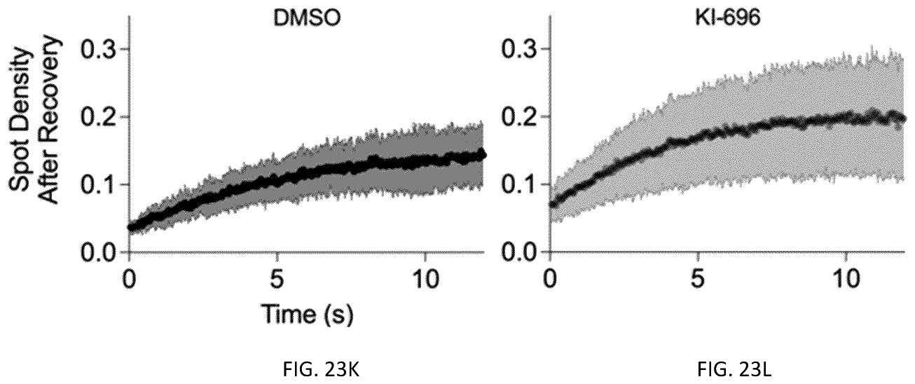

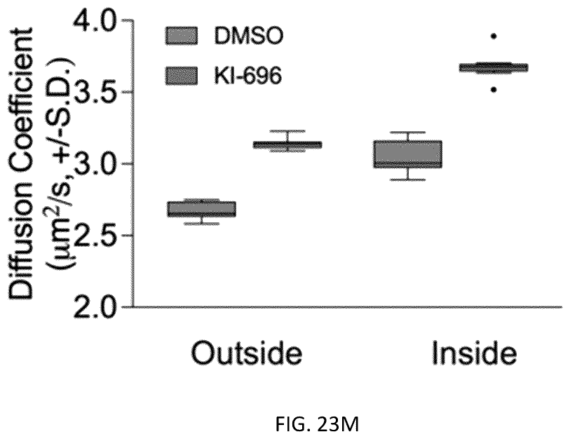

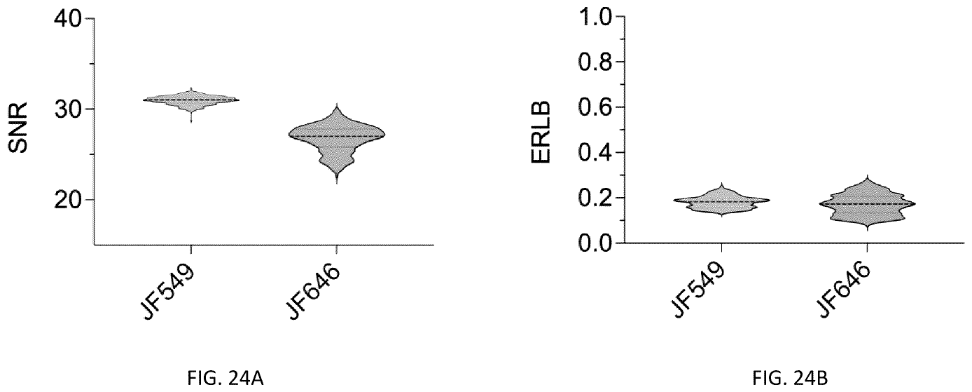

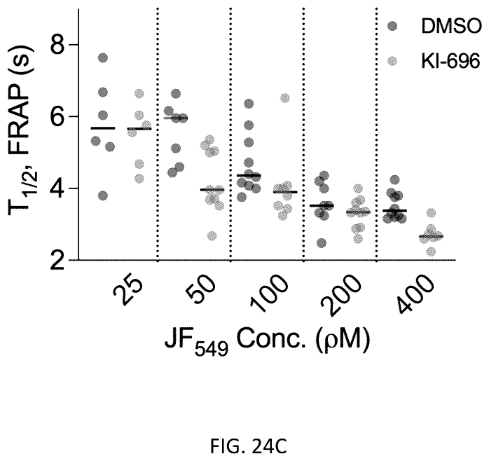

The homogenous illumination, high spatiotemporal resolution, imaging speed and overall robustness of the OLS system likely have broad advantages across multiple biological microscopy techniques. To demonstrate the ability to image SMT across two spectrally distinct fluorophores, both JF₅₄₉ and JF₆₄₆-labeled Halo-Keap1 time series were captured sequentially resulting in clear single molecule resolution across both wavelengths (FIG. 23A). KI-696 was dose titrated and the response was measured across both JF₅₄₉ and JF₆₄₆-labeled Halo-Keap1 to demonstrate the ability to measure changes in protein motion across two wavelengths (FIG. 23B). Despite a drop in sCMOS quantum efficiency in the far-red spectrum resulting in lower SNR (FIG. 24A), SMT data was captured using the red-shifted JF₆₄₆ that results in the EC₅₀ values that were highly consistent with the brighter JF₅₄₉ dye with measured EC₅₀s of 4.96 nM and 6.45 nM for JF₅₄₉ and JF₆₄₆, respectively. Robust measurement of protein dynamics with lower SNR is potentially explained by minimal decrease in lower bound error rate (ERLB) (FIG. 24B). Taken together, OLS-based SMT is likely to have applications in multi-color imaging and facilitates imaging of lower quantum yield fluorophores, broadening the palette of available fluorophores to measure protein motion across biological applications.

Due to the short integration time resulting from the OLS line scan, it was investigated whether dyes typically used for STORM imaging in fixed cells would result in images with high x,y resolution across a large FOV. Cells labeled with anti-tubulin primary antibodies and AlexaFluor647 (AF647) or CF568-conjugated secondary antibodies, were imaged using STORM across a full 60× field of view with a total imaging time of ˜60 seconds (FIGS. 23C-23F). It was observed that despite the low integration time of 400 μs used in OLS, spontaneous photoswitching provided a sufficient number of photons were collected to achieve a lateral localization precision of approximately 15 nm using either AF647 or CF568 (FIGS. 23G and 23H). These results demonstrate the opportunity of conducting high-speed high-throughput phenotypic screening with STORM or other super-resolution microscopy techniques with OLS illumination.

Next, the line scan component of OLS was leveraged to conduct a correlative SMT/FRAP experiment on KI-696 treated Halo-KEAP1 cells labeled with JF₅₄₉. A 240×40 μm region was bleached by focusing the scan region on a narrow subset of the FOV for a duration of 100-200 sweeps prior to acquiring the full FOV under normal SMT acquisitions. This resulted in each frame consisting of a bleached (FRAP region) and an unbleached region (FIG. 23I). Instead of capturing the intensity recovery after photobleaching over time, the normalized spot density after recovery was measured (FIGS. 23K and 23L). An average T_{1/2}(DMSO) of 3.52 (SD=0.38) and T_{1/2}(KI-696) of 2.27 (SD=0.43) at a JF₅₄₉ dye concentration of 400 pM was measured that appears optimal to separate the two conditions (FIG. 23J). This result is consistent with the reported SMT measurements and indicates that KI-696 significantly increases Halo-KEAP1 local protein dynamics. To further ascertain the consistency of the FRAP measurement, SMT was applied to both bleached and unbleached regions of the FOV. In both cases, an increase in Halo-KEAP1 dynamics was measured under a 1 μM KI-696 perturbation range of dye concentration (FIGS. 23M and 24C). These results highlight and confirm that the advantageous properties brought upon by OLS in combination with the presented tracking algorithm enable sensitive spot detection and SMT.

### B. Methods

### a. 384 Well-Plate Coating and Cell Seeding for SMT

Cells were seeded in tissue culture treated 384-well glass-bottom plates (Cellvis #1.5 cover glass) at 4000-6000 cells per well and allowed to adhere and incubate overnight at 37° C. and 5% CO₂. Cells were labeled for SMT using JF₅₄₉ and JF₆₄₆ along with Hoechst 33342 and organelle specific dyes for an hour prior to washing with DPBS (3×) and Fluorobrite DMEM media (2×).

b. Line FRAP Acquisition

FRAP datasets U2OS-KEAP1 were recorded by consecutively imaging 5 pre-bleaching frames, bleaching a subregion of the imaging FOV, and capturing fluorescence recovery at an operating framerate of 25 fps. Bleaching a FOV subregion was achieved by scanning the excitation laser for 100-200 sweeps across a subregion of the FOV (16-25% FOV height, 100% FOV width) at high laser power (300 mW) in order to bleach the local fluorescence. Fluorescence recovery was acquired with 400-500 frames following the bleaching step at low laser power (70 mW). Modulating the laser power was achieved by implementing an Acousto-Optical Tunable Filter in the laser engine module (AOTF-Ed 2018-1; Opto-Electronic) adjusted for a bleaching and imaging power of the system's 560 nm-excitation.

c. STORM and PALM Datasets

U2OS cells were fixed in 4% PFA for 10 minutes, washed three times with PBS, permeabilized with 0.1% Triton-X for 10 minutes and washed again three times with PBS. Cells were then blocked in 2% BSA for 1 hour at room temperature followed by overnight incubation with anti-alpha tubulin antibody (ab7291) at 4° C. The primary antibody was removed by washing three times with PBS and cells were blocked again with 2% BSA for 1 hour at room temperature. Alexa Fluorophore 647-conjugated secondary antibodies were diluted 1:1000 in blocking buffer and incubated for 2 hours at room temperature. Cells were then washed three times with PBS to remove excess secondary antibody. Hoechst 33342 was added at 7 μM concentration with the secondary antibodies for nuclei visualization. Photoswitching buffer for STORM imaging was prepared as previously described. Buffer A was prepared with 0.5 mL 1M Tris (pH 8.0), 0.146 g NaCl and 50 mL of H₂O. Buffer B with 2.5 mL 1M Tris (pH 8.0), 0.029 g NaCl, 5 g Glucose and 47.5 mL of H₂O. GLOX solution was prepared by mixing 14 mg Glucose Oxidase (G2133) and 17 mg/mL of Catalase (C40) with 200 μL of Buffer A. The final photoswitching buffer was prepared by combining 100 μL 1M MEA (M9768) with 10 μL of GLOX and 1 mL of buffer B. This buffer was added to cells in the 384 well plate prior to imaging.

Low laser power (300 mW) was used to capture diffraction-limited images and identify the pertinent focal plane. Laser power was increased to achieve ˜500 mW (x kW/cm²) at the back focal plane in order to initiate photoswitching. 500-5000 frames were acquired, with a pixel integration time of 0.4 ms.

### C. Discussion

These results highlight and confirm that the advantageous properties brought upon by OLS in combination with the presented tracking algorithm enable sensitive spot detection and SMT even when labeling sparsity was not optimized. In particular, these results highlight the versatility of OLS by demonstrating its suitability for rapidly capturing STORM datasets despite very low illumination integration times and achieving a lateral resolution of ˜30 nm for both AF647 and CF568. The results further demonstrate a correlative SMT/FRAP approach by which to study protein diffusion.

The example demonstrates that the large FOV, OLS scanning, and high SNR with fast integration times enabled 2 color SMT, STORM and FRAP to be implemented with scale, speed and consistency. It is anticipated that this platform will be compatible with other approaches such as fluorescence correlation spectroscopy (FCS) and image correlation spectroscopy (ICS) and could enable leveraging such advanced microscopy methods in high content applications within a systems biology and drug screening.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. Accordingly, the appended claims are intended to comprise within their scope such processes, machines, manufacture, compositions of matter, means, methods or steps.

Various patents, patent applications, publications, product descriptions and protocols are cited throughout this application, the disclosure of which are incorporated herein by reference in their entireties for all purposes.

1. A method of identifying a biological interaction between a compound and a target fluorescent protein in a live cell comprising:
(a) contacting a sample comprising a population of live cells with the compound, wherein the live cells comprise target fluorescent protein;
(b) tracking the movement of a plurality of individual target fluorescent proteins in a plurality of live cells in the sample, wherein said tracking comprises:
(i) illuminating a field of view in a sample plane disposed within the sample with a light beam to cause fluorescence by a subset of the target fluorescent proteins in the live cells; and
(ii) detecting the fluorescence from a plurality of the target fluorescent proteins in a detected field of view of the sample plane via a detector device, wherein the detected field of view has a size of about 150 μm to about 250 μm in a first dimension by about 100 μm to about 210 μm in a second dimension; and
(c) determining a change in the movement of at a target fluorescent protein in the presence of the compound,
wherein a change in the movement of the target fluorescent protein in the presence of the compound relative to the movement of the target fluorescent protein in the absence of the compound identifies a biological interaction between the compound and the target fluorescent protein.

2. The method of claim 1, wherein the method is characterized by one or more of the following:
(i) the subset of the target fluorescent proteins produces up to about 1,000,000 molecular trajectories in a single detected field of view;
(ii) the field of view is illuminated using a 0.1 to 1 msec stroboscopic laser pulse;
(iii) the fluorescence from a plurality of the target fluorescent proteins in the detected field of view of the sample plane is detected at a rate of more than about 100,000 detected field of views per day;
(iv) achieving, based on a single field of view, a z-factor of >0.5;
(v) the field of view is detected with a frame rate up to about 2000 Hz;
(vi) equal to or greater than 95% of the detected field of view achieves sufficient laser illumination for tracking protein movement; and
(vii) the average change in movement of the target fluorescent proteins in the presence of the compound is at least 1%, at least 5%, or at least 10% relative to the change observed in the absence of the compound.

3.-9. (canceled)

10. The method of claim 1, wherein;
(i) the change in movement is detected as an increase in immobile trajectories indicating an increase in bound (f_{bound}) target fluorescent protein; and/or
(ii) the change in movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(i) state occupation via inference.

11. The method of claim 1, wherein the subset of the target fluorescent proteins are in up to about 80 live cells illuminated in the field of view of the sample plane.

12. The method of claim 1, wherein the target fluorescent protein interacts in a larger molecular assembly.

13. The method of claim 12, wherein the target fluorescent protein is a ligand or a receptor.

14. (canceled)

15. The method of claim 1, wherein the biological interaction is a direct interaction or an indirect interaction.

16. The method of claim 15, wherein the direct interaction comprises binding of the compound to the target fluorescent protein or the indirect interaction comprises the compound agonizing or antagonizing a larger molecular assembly comprising the target fluorescent protein.

17. (canceled)

18. (canceled)

19. A method of determining a dose response of a compound that induces a change in the movement of a target fluorescent protein in a live cell comprising:
(a) contacting a plurality of samples with the compound,
(i) wherein each sample comprises a population of live cells;
(ii) wherein the live cells comprise target fluorescent protein; and
(iii) wherein the plurality of samples are contacted with distinct concentrations of the compound across a range of compound concentrations;
(b) tracking the movement of individual target fluorescent proteins in a plurality of live cells of a sample, wherein said tracking comprises:
(i) illuminating a field of view in a sample plane disposed within the sample with a light beam to cause fluorescence by at least a subset of the target fluorescent proteins in the live cells; and
(ii) detecting the fluorescence from one or more of the target fluorescent proteins in a detected field of view of the sample plane via a detector device, wherein the detected field of view has a size of about 150 μm to about 250 μm in a first dimension by about 100 μm to about 210 μm in a second dimension; and
(c) determining a change in the movement of a target fluorescent protein in the presence of the compound; and
(d) repeating steps (b)-(c) for each of the plurality of samples across the range of compound concentrations,
wherein a change in the movement of the target fluorescent protein in the presence of the compound across the concentration range indicates the dose response of the compound.

20. The method of claim 19, wherein the method is characterized by one or more of the following:
(i) the subset of the target fluorescent proteins produces up to about 1,000,000 molecular trajectories in a single detected field of view;
(ii) the field of view is illuminated using a 0.1 to 1 msec stroboscopic laser pulse;
(iii) the fluorescence from a plurality of the target fluorescent proteins in the detected field of view of the sample plane is detected at a rate of more than about 100,000 detected field of views per day;
(iv) achieving, based on a single field of view, a z-factor of >0.5;
(v) the field of view is detected with a frame rate up to about 2000 Hz;
(vi) equal to or greater than 95% of the detected field of view achieves sufficient laser illumination for tracking protein movement; and
(v) the average change in movement of the target fluorescent proteins in the presence of the compound is at least 1%, at least 5%, or at least 10% relative to the change observed in the absence of the compound.

21.-27. (canceled)

28. The method of claim 19, wherein;
(i) the change in movement detected is an increase in immobile trajectories indicating an increase in bound (f_{bound}) target fluorescent protein; and/or
(ii) the movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(i) state occupation via inference.

29. The method of claim 19, wherein the subset of the target fluorescent proteins are in up to about 80 live cells illuminated in the field of view of the sample plane.

30. The method of claim 19, wherein the target fluorescent protein interacts in a larger molecular assembly and/or the biological interaction is a direct interaction or an indirect interaction.

31. The method of claim 30, wherein the target fluorescent protein is a ligand or a receptor.

32.-36. (canceled)

37. A microscopy system configured to identify a biological interaction between a compound and a target fluorescent protein in a live cell comprising:
(a) a stage for supporting a sample, wherein the sample comprises a population of live cells, and wherein the live cells comprise the target fluorescent protein;
(b) a light source for emitting a light beam capable of inducing a light-based response from a plurality of the target fluorescent proteins in the sample;
(c) an objective for focusing the light beam on the sample in the sample plane, wherein a subset of the target fluorescent proteins in the sample are disposed in a detected field of view of the sample plane and wherein the detected field of view has a size of about 150 μm to about 250 μm in a first dimension by about 100 μm to about 210 μm in a second dimension;
(d) a detector device for monitoring the light-based response from the target fluorescent proteins in the presence of the compound;
(e) a memory; and
(f) a processor in communication with the memory and the detector device, wherein the processor is capable of determining the change in the movement of the target fluorescent protein in the presence of the compound relative to the absence of the compound.

38. The microscopy system of claim 37, wherein the system is characterized by one or more of the following:
(i) the subset of the target fluorescent proteins produces up to about 1,000,000 molecular trajectories in a single field of view;
(ii) the field of view is illuminated using a 0.1 to 1 msec stroboscopic laser pulse;
(iii) said monitoring comprises detecting the fluorescence from a plurality of the target fluorescent proteins in the detected field of view of the sample plane at a rate of more than about 100,000 detected field of views per day;
(iv) the system has a z-factor of >0.5 based on a single detected field of view;
(v) the detector device has a frame rate up to about 2000 Hz;
(vi) equal to or greater than 95% of the detected field of view achieves sufficient laser illumination for tracking protein movement; and
(v) the average change in movement of the target fluorescent proteins in the presence of the compound is at least 1%, at least 5%, or at least 10% relative to the change observed in the absence of the compound.

39.-45. (canceled)

46. The system of claim 37, wherein;
(i) the change in movement detected is an increase in immobile trajectories indicating an increase in bound (f_{bound}) target fluorescent protein; and/or
(ii) the movement detected is a change in:
(a) the median of the jump length distribution;
(b) 3^{rd} quartile of the jump length distribution;
(c) median radius of gyration;
(d) mean posterior diffusion coefficient;
(e) geometric mean posterior diffusion coefficient;
(f) mean squared displacement;
(g) median bond angle;
(h) diffusion coefficient maximum likelihood estimator; and/or
(i) state occupation via inference.

47. The system of claim 37, wherein the subset of the target fluorescent proteins are in up to about 80 live cells illuminated in the field of view of the sample plane.

48. The system of claim 37, wherein the target fluorescent protein interacts in a larger molecular assembly and/or the biological interaction is a direct interaction or an indirect interaction.

49. The system of claim 48, wherein the target fluorescent protein is a ligand or a receptor.

50. (canceled)

51. (canceled)

52. The system of claim 51, wherein the direct interaction comprises binding of the compound to the target fluorescent protein or the indirect interaction comprises the compound agonizing or antagonizing a larger molecular assembly comprising the target fluorescent protein.

53. (canceled)

54. (canceled)
